(19) United States
(12) Patent Application Publication
(10) Pub. No.: US 2006/0281768 A1
(43) Pub. Date: Dec. 14, 2006

(54) THIENOPYRIMIDINE AND THIENOPYRIDINE KINASE MODULATORS

(76) Inventors: Michael David Gaul, Yardley, PA (US); Kevin Douglas Kreutter, Plainsboro, NJ (US); Christian Andrew Baumann, Exton, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/422,359

(22) Filed: Jun. 6, 2006

Related U.S. Application Data
(60) Provisional application No. 60/689,710, filed on Jun. 10, 2005.
(60) Provisional application No. 60/746,941, filed on May 10, 2006.

(51) Int. Cl. A61K 31/519; A61K 31/4743; C07D 498/02

(52) U.S. Cl. 514/260.1; 514/301; 544/278; 546/114

(57) ABSTRACT

The invention is directed to thienopyrimidines and thienopyridines compounds of Formula I and Formula II: 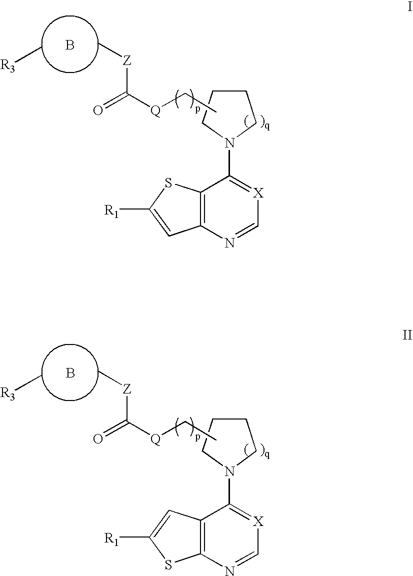
where R₁, R₃, B, Z, Q, p, q and X are as defined herein, the use of such compounds as protein tyrosine kinase modulators, particularly inhibitors of FLT3, the use of such compounds to reduce or inhibit kinase activity of FLT3 in a cell or a subject, and the use of such compounds for preventing or treating in a subject a cell proliferative disorder and/or disorders related to FLT3. The present invention is further directed to pharmaceutical compositions comprising the compounds of the present invention and to methods for treating conditions such as cancers and other cell proliferative disorders.

### CROSS REFERENCE TO RELATED APPLICATIONS

This application claims priority to U.S. Provisional Application for Patent No. 60/689,710, filed Jun. 10, 2005, and U.S. Provisional Application for Patent No. 60/746,941, filed May 10, 2006, the entire disclosures of which are hereby incorporated in their entirely.

### FIELD OF THE INVENTION

The invention relates to novel compounds that function as protein tyrosine kinase modulators. More particularly, the invention relates to novel compounds that function as inhibitors of FLT3.

### BACKGROUND OF THE INVENTION

The present invention relates to thienopyrimidines and thineopyridines as inhibitors of tyrosine kinases, including FLT3. Thiophenes and other compounds reported with useful therapeutic properties include: WO2004/016576 (heterocyclic moiety-containing fused benzene derivatives as androgen receptor modulators); WO2002/0705 10 and US 2004082798 (aminodicarboxylic acids for the treatment of cardiovascular diseases); WO 9605828 ((2R,4R)-4-Aminopyrrolidine-2,4-dicarboxylic acid derivatives as metabotropic glutamate receptor antagonists); WO 9852906, U.S. Pat. No. 5,932,765, U.S. Pat. No. 6,043,281, US 2003069312, U.S. Pat. No. 6,559,185 and US 2003171422 (nitromethyl ketones for use as aldose reductase inhibitors); WO 9937304, WO 2000032590 and US 2004102450 (substituted piperazinone derivatives and other oxoazaheterocyclyl compounds useful as factor Xa inhibitors); WO 2003088967 and US 2004097483 (1-(4-piperidinyl)benzimidazoles as histamine H3 antagonists); WO 9209567 (hydroxamic acid derivatives as potential antiinflammatory compounds).

Protein kinases are enzymatic components of the signal transduction pathways which catalyze the transfer of the terminal phosphate from ATP to the hydroxy group of tyrosine, serine and/or threonine residues of proteins. Thus, compounds which inhibit protein kinase functions are valuable tools for assessing the physiological consequences of protein kinase activation. The overexpression or inappropriate expression of normal or mutant protein kinases in mammals has been a topic of extensive study and has been demonstrated to play a significant role in the development of many diseases, including diabetes, angiogenesis, psoriasis, restenosis, ocular diseases, schizophrenia, rheumatoid arthritis, atherosclerosis, cardiovascular disease and cancer. The cardiotonic benefits of kinase inhibition has also been studied. In sum, inhibitors of protein kinases have particular utility in the treatment of human and animal disease.

The fms-like tyrosine kinase 3 (FLT3) ligand (FLT3L) is one of the cytokines that affects the development of multiple hematopoietic lineages. These effects occur through the binding of FLT3L to the FLT3 receptor, also referred to as fetal liver kinase-2 (flk-2) and STK- 1, a receptor tyrosine kinase (RTK) expressed on hematopoietic stem and progenitor cells. The FLT3 gene encodes a membrane-bound RTK that plays an important role in proliferation, differentiation and apoptosis of cells during normal hematopoiesis. The FLT3 gene is mainly expressed by early meyloid and lymphoid progenitor cells. See McKenna, Hilary J. et al. Mice lacking flt3 ligand have deficient hematopoiesis affecting hematopoietic progenitor cells, dendritic cells, and natural killer cells. Blood. Jun 2000; 95: 3489-3497; Drexler, H. G. and H. Quentmeier (2004). “FLT3: receptor and ligand.” Growth Factors 22(2): 71-3.

The ligand for FLT3 is expressed by the marrow stromal cells and other cells and synergizes with other growth factors to stimulate proliferation of stem cells, progenitor cells, dendritic cells, and natural killer cells.

Hematopoietic disorders are pre-malignant disorders of these systems and include, for instance, the myeloproliferative disorders, such as thrombocythemia, essential thrombocytosis (ET), angiogenic myeloid metaplasia, myelofibrosis (MF), myelofibrosis with myeloid metaplasia (MMM), chronic idiopathic myelofibrosis (IMF), and polycythemia vera (PV), the cytopenias, and pre-malignant myelodysplastic syndromes. See Stirewalt, D. L. and J. P. Radich (2003). “The role of FLT3 in haematopoietic malignancies.” Nat Rev Cancer 3(9): 650-65; Scheijen, B. and J. D. Griffin (2002). “Tyrosine kinase oncogenes in normal hematopoiesis and hematological disease.” Oncogene 21(21): 3314-33.

Hematological malignancies are cancers of the body's blood forming and immune systems, the bone marrow and lymphatic tissues. Whereas in normal bone marrow, FLT3 expression is restricted to early progenitor cells, in hematological malignancies, FLT3 is expressed at high levels or FLT3 mutations cause an uncontrolled induction of the FLT3 receptor and downstream molecular pathway, possibly Ras activation. Hematological malignancies include leukemias, lymphomas (non-Hodgkin's lymphoma), Hodgkin's disease (also called Hodgkin's lymphoma), and myeloma—for instance, acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myelodysplastic syndromes (MDSs), myeloproliferative disorders (MPD), multiple myeloma, (MM) and myeloid sarcoma. See Kottaridis, P. D., R. E. Gale, et al. (2003). “Flt3 mutations and leukaemia.” Br J Haematol 122(4): 523-38. Myeloid sarcoma is also associated with FLT3 mutations. See Ansari-Lari, Ali et al. FLT3 mutations in myeloid sarcoma. British Journal of Haematology. 2004 September 126(6):785-91.

Mutations of FLT3 have been detected in about 30% of patients with acute myelogenous leukemia and a small number of patients with acute lymphomatic leukemia or myelodysplastic syndrome. Patients with FLT3 mutations tend to have a poor prognosis, with decreased remission times and disease free survival. There are two known types of activating mutations of FLT3. One is a duplication of 4-40 amino acids in the juxtamembrane region (ITD mutation) of the receptor (25-30% of patients) and the other is a point mutation in the kinase domain (5-7% of patients). The mutations most often involve small tandem duplications of amino acids within the juxtamembrane domain of the receptor and result in tyrosine kinase activity. Expression of a mutant FLT3 receptor in murine marrow cells results in a lethal myeloproliferative syndrome, and preliminary studies (Blood. 2002; 100: 1532-42) suggest that mutant FLT3 cooperates with other leukemia oncogenes to confer a more aggressive phenotype.

Taken together, these results suggest that specific inhibitors of the individual kinase FLT3, present an attractive target for the treatment of hematopoietic disorders and hematological malignancies.

FLT3 kinase inhibitors known in the art include AG1295 and AG1296; Lestaurtinib (also known as CEP 701, formerly KT-5555, Kyowa Hakko, licensed to Cephalon); CEP-5214 and CEP-7055 (Cephalon); CHIR-258 (Chiron Corp.); EB-10 and IMC-EB10 (ImClone Systems Inc.); GTP 14564 (Merk Biosciences UK). Midostaurin (also known as PKC 412 Novartis AG); MLN 608 (Millennium USA); MLN-518 (formerly CT53518, COR Therapeutics Inc., licensed to Millennium Pharmaceuticals Inc.); MLN-608 (Millennium Pharmaceuticals Inc.); SU-1 1248 (Pfizer USA); SU-11657 (Pfizer USA); SU-5416 and SU 5614; THRX-165724 (Theravance Inc.); AMI-10706 (Theravance Inc.); VX-528 and VX-680 (Vertex Pharmaceuticals USA, licensed to Novartis (Switzerland), Merck & Co USA); and XL 999 (Exelixis USA). The following PCT International Applications and US Patent Applications disclose additional kinase modulators, including modulators of FLT3: WO 2002032861, WO 2002092599, WO 2003035009, WO 2003024931, WO 2003037347, WO 2003057690, WO 2003099771, WO 2004005281, WO 2004016597, WO 2004018419, WO 2004039782, WO 2004043389, WO 2004046120, WO 2004058749, WO 2004058749, WO 2003024969 and US Patent Application No. 20040049032.

See also Levis, M., K. F. Tse, et al. 2001 “A FLT3 tyrosine kinase inhibitor is selectively cytotoxic to acute myeloid leukemia blasts harboring FLT3 internal tandem duplication mutations.” Blood 98(3): 885-7; Tse KF, et al. Inhibition of FLT3-mediated transformation by use of a tyrosine kinase inhibitor. Leukemia. 2001 July; 15(7): 1001-10; Smith, B. Douglas et al. Single-agent CEP-701, a novel FLT3 inhibitor, shows biologic and clinical activity in patients with relapsed or refractory acute myeloid leukemia Blood, May 2004; 103: 3669-3676; Griswold, Ian J. et al. Effects of MLN518, A Dual FLT3 and KIT Inhibitor, on Normal and Malignant Hematopoiesis. Blood, Jul 2004; [Epub ahead of print]; Yee, Kevin W. H. et al. SU5416 and SU5614 inhibit kinase activity of wild-type and mutant FLT3 receptor tyrosine kinase. Blood, Sep 2002; 100: 2941-294; O° Farrell, Anne-Marie et al. SU11248 is a novel FLT3 tyrosine kinase inhibitor with potent activity in vitro and in vivo. Blood, May 2003; 101: 3597-3605; Stone, R.M. et al. PKC 412 FLT3 inhibitor therapy in AML: results of a phase II trial. Ann Hematol. 2004; 83 Suppl 1:S89-90; and Murata, K. et al. Selective cytotoxic mechanism of GTP-14564, a novel tyrosine kinase inhibitor in leukemia cells expressing a constitutively active Fms-like tyrosine kinase 3 (FLT3). J Biol Chem. 2003 Aug 29; 278(35):32892-8; Levis, Mark et al. Novel FLT3 tyrosine kinase inhibitors. Expert Opin. Investing. Drugs (2003) 12(12) 1951-1962; Levis, Mark et al. Small Molecule FLT3 Tyrosine Kinase Inhibitors. Current Pharmaceutical Design, 2004, 10, 1183-1193.

### SUMMARY OF THE INVENTION

The present invention provides novel thienopyrimidines and thineopyridines (the compounds of Formula I and Formula II) as protein tyrosine kinase modulators, particularly inhibitors of FLT3, and the use of such compounds to reduce or inhibit kinase activity of FLT3 in a cell or a subject, and the use of such compounds for preventing or treating in a subject a cell proliferative disorder and/or disorders related to FLT3.

Illustrative of the invention is a pharmaceutical composition comprising a compound of Formula I or Formula II and a pharmaceutically acceptable carrier. Another illustration of the present invention is a pharmaceutical composition prepared by mixing any of the compounds of Formula I and Formula II and a pharmaceutically acceptable carrier.

Other features and advantages of the invention will be apparent from the following detailed description of the invention and from the claims.

### DETAILED DESCRIPTION OF THE INVENTION

Definitions

As used herein, the following terms are intended to have the following meanings (additional definitions are provided where needed throughout the Specification):

The term “alkenyl,” whether used alone or as part of a substituent group, for example, “C₁₋₄alkenyl(aryl),” refers to a partially unsaturated branched or straight chain monovalent hydrocarbon radical having at least one carbon-carbon double bond, whereby the double bond is derived by the removal of one hydrogen atom from each of two adjacent carbon atoms of a parent alkyl molecule and the radical is derived by the removal of one hydrogen atom from a single carbon atom. Atoms may be oriented about the double bond in either the cis (Z) or trans (E) conformation. Typical alkenyl radicals include, but are not limited to, ethenyl, propenyl, allyl (2-propenyl), butenyl and the like. Examples include C₂₋₈alkenyl or C₂₋₄alkenyl groups.

The term “Ca_{a-b}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₁₄ denotes a radical containing 1, 2, 3 or 4 carbon atoms.

The term “alkyl,” whether used alone or as part of a substituent group, refers to a saturated branched or straight chain monovalent hydrocarbon radical, wherein the radical is derived by the removal of one hydrogen atom from a single carbon atom. Unless specifically indicated (e.g. by the use of a limiting term such as “terminal carbon atom”), substituent variables may be placed on any carbon chain atom. Typical alkyl radicals include, but are not limited to, methyl, ethyl, propyl, isopropyl and the like. Examples include C₁₋₈alkyl, C₁₋₆alkyl and C₁₋₄alkyl groups.

The term “alkylamino” refers to a radical formed by the removal of one hydrogen atom from the nitrogen of an alkylamine, such as butylamine, and the term “dialkylamino” refers to a radical formed by the removal of one hydrogen atom from the nitrogen of a secondary amine, such as dibutylamine. In both cases it is expected that the point of attachment to the rest of the molecule is the nitrogen atom.

The term “alkynyl,” whether used alone or as part of a substituent group, refers to a partially unsaturated branched or straight chain monovalent hydrocarbon radical having at least one carbon-carbon triple bond, whereby the triple bond is derived by the removal of two hydrogen atoms from each of two adjacent carbon atoms of a parent alkyl molecule and the radical is derived by the removal of one hydrogen atom from a single carbon atom. Typical alkynyl radicals include ethynyl, propynyl, butynyl and the like. Examples include C₂₋₈alkynyl or C₂₋₄alkynyl groups.

The term “alkoxy” refers to a saturated or partially unsaturated branched or straight chain monovalent hydrocarbon alcohol radical derived by the removal of the hydrogen atom from the hydroxide oxygen substituent on a parent alkane, alkene or alkyne. Where specific levels of saturation are intended, the nomenclature “alkoxy”, “alkenyloxy” and “alkynyloxy” are used consistent with the definitions of alkyl, alkenyl and alkynyl. Examples include C₁₋₈alkoxy or C₁₋₄alkoxy groups.

The term “alkoxyether” refers to a saturated branched or straight chain monovalent hydrocarbon alcohol radical derived by the removal of the hydrogen atom from the hydroxide oxygen substituent on a hydroxyether. Examples include 1-hydroxyl-2-methoxy-ethane and 1-(2-hydroxyl-ethoxy)-2-methoxy-ethane groups.

The term “aralkyl” refers to a C₁₆ alkyl group containing an aryl substituent. Examples include benzyl, phenylethyl or 2-naphthylmethyl. It is intended that the point of attachment to the rest of the molecule be the alkyl group.

The term “aromatic” refers to a cyclic hydrocarbon ring system having an unsaturated, conjugated 7r electron system.

The term “aryl” refers to an aromatic cyclic hydrocarbon ring radical derived by the removal of one hydrogen atom from a single carbon atom of the ring system. Typical aryl radicals include phenyl, naphthalenyl, fluorenyl, indenyl, azulenyl, anthracenyl and the like.

The term “arylamino” refers to an amino group, such as ammonia, substituted with an aryl group, such as phenyl. It is expected that the point of attachment to the rest of the molecule is through the nitrogen atom.

The term “benzo-fused cycloalkyl” refers to a bicyclic fused ring system radical wherein one of the rings is phenyl and the other is a cycloalkyl or cycloalkenyl ring. Typical benzo-fused cycloalkyl radicals include indanyl, 1,2,3,4-tetrahydro-naphthalenyl, 6,7,8,9,-tetrahydro-5H-benzocycloheptenyl, 5,6,7,8,9,1 0-hexahydro-benzocyclooctenyl and the like. A benzo-fused cycloalkyl ring system is a subset of the aryl group.

The term “benzo-fused heteroaryl” refers to a bicyclic fused ring system radical wherein one of the rings is phenyl and the other is a heteroaryl ring. Typical benzo-fused heteroaryl radicals include indolyl, indolinyl, isoindolyl, benzo[b]furyl, benzo[b]thienyl, indazolyl, benzthiazolyl, quinolinyl, isoquinolinyl, cinnolinyl, phthalazinyl, quinazolinyl, and the like. A benzo-fused heteroaryl ring is a subset of the heteroaryl group.

The term “benzo-fused heterocyclyl” refers to a bicyclic fused ring system radical wherein one of the rings is phenyl and the other is a heterocyclyl ring. Typical benzo-fused heterocyclyl radicals include 1,3-benzodioxolyl (also known as 1,3-methylenedioxyphenyl), 2,3-dihydro-1,4-benzodioxinyl (also known as 1,4-ethylenedioxyphenyl), benzo-dihydro-furyl, benzo-tetrahydro-pyranyl, benzo-dihydro-thienyl and the like.

The term “carboxyalkyl” refers to an alkylated carboxy group such as tert-butoxycarbonyl, in which the point of attachment to the rest of the molecule is the carbonyl group.

The term “cyclic heterodionyl” refers to a heterocyclic compound bearing two carbonyl substituents. Examples include thiazolidine dionyls, oxazolidine dionyls and pyrrolidine dionyls.

The term “cycloalkenyl” refers to a partially unsaturated cycloalkyl radical derived by the removal of one hydrogen atom from a hydrocarbon ring system that contains at least one carbon-carbon double bond. Examples include cyclohexenyl, cyclopentenyl and 1,2,5,6-cyclooctadienyl.

The term “cycloalkyl” refers to a saturated or partially unsaturated monocyclic or bicyclic hydrocarbon ring radical derived by the removal of one hydrogen atom from a single ring carbon atom. Typical cycloalkyl radicals include cyclopropyl, cyclobutyl, cyclopentyl, cyclopentenyl, cyclohexyl, cyclohexenyl, cycloheptyl and cyclooctyl. Additional examples include C₃₋₈cycloalkyl, C₅₋₈cycloalkyl, C₃₋₁₂cycloalkyl, C₃₋₂₀cycloalkyl, decahydronaphthalenyl, and 2,3,4,5,6,7-hexahydro-1H-indenyl.

The term “fused ring system” refers to a bicyclic molecule in which two adjacent atoms are present in each of the two cyclic moieties. Heteroatoms may optionally be present. Examples include benzothiazole, 1,3-benzodioxole and decahydronaphthalene.

The term “hetero” used as a prefix for a ring system refers to the replacement of at least one ring carbon atom with one or more atoms independently selected from N, S, o or P. Examples include rings wherein 1, 2, 3 or 4 ring members are a nitrogen atom; or, 0, 1, 2 or 3 ring members are nitrogen atoms and 1 member is an oxygen or sulfur atom.

The term “heteroaralkyl” refers to a C₁₆ alkyl group containing a heteroaryl substituent. Examples include furylmethyl and pyridylpropyl. It is intended that the point of attachment to the rest of the molecule be the alkyl group.

The term “heteroaryl” refers to a radical derived by the removal of one hydrogen atom from a ring carbon atom of a heteroaromatic ring system. Typical heteroaryl radicals include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolizinyl, indolyl, isoindolyl, benzo[b]furyl, benzo[b]thienyl, indazolyl, benzimidazolyl, benzthiazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl and the like.

The term “heteroaryl-fused cycloalkyl” refers to a bicyclic fused ring system radical wherein one of the rings is cycloalkyl and the other is heteroaryl. Typical heteroaryl-fused cycloalkyl radicals include 5,6,7,8-tetrahydro-4H-cyclohepta(b)thienyl, 5,6,7-trihydro-4H-cyclohexa(b)thienyl, 5,6-dihydro-4H-cyclopenta(b)thienyl and the like.

The term “heterocyclyl” refers to a saturated or partially unsaturated monocyclic ring radical derived by the removal of one hydrogen atom from a single carbon or nitrogen ring atom. Typical heterocyclyl radicals include 2H-pyrrole, 2-pyrrolinyl, 3-pyrrolinyl, pyrrolidinyl, 1,3-dioxolanyl, 2-imidazolinyl (also referred to as 4,5-dihydro-1H-imidazolyl), imidazolidinyl, 2-pyrazolinyl, pyrazolidinyl, tetrazolyl, piperidinyl, 1,4-dioxanyl, morpholinyl, 1,4-dithianyl, thiomorpholinyl, piperazinyl, azepanyl, hexahydro-1,4-diazepinyl and the like. pyrrolinyl, The term “substituted,” refers to a core molecule on which one or more hydrogen atoms have been replaced with one or more functional radical moieties. Substitution is not limited to a core molecule, but may also occur on a substituent radical, whereby the substituent radical becomes a linking group. pyrrolinyl, The term “independently selected” refers to one or more substituents selected from a group of substituents, wherein the substituents may be the same or different. pyrrolinyl, The substituent nomenclature used in the disclosure of the present invention was derived by first indicating the atom having the point of attachment, followed by the linking group atoms toward the terminal chain atom from left to right, substantially as in:
(C₁₋₆)alkylC(O)NH(C ₁₋₆)alkyl(Ph)
or by first indicating the terminal chain atom, followed by the linking group atoms toward the atom having the point of attachment, substantially as in:
Ph(C₁₋₆)alkylamido(C₁₆)alkyl
either of which refers to a radical of the formula:

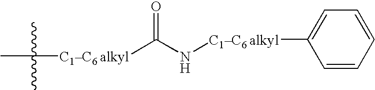

Lines drawn into ring systems from substituents indicate that the bond may be attached to any of the suitable ring atoms.

When any variable (e.g. R₄) occurs more than one time in any embodiment of Formula I or Formula II, each definition is intended to be independent.

The terms “comprising”, “including”, and “containing” are used herein in their open, non-limited sense.

Nomenclature

Except where indicated, compound names were derived using nomenclature rules well known to those skilled in the art, by either standard IUPAC nomenclature references, such as Nomenclature of Organic Chemistry, Sections A, B, C, D, E, F and H

, (Pergamon Press, Oxford, 1979, Copyright 1979 IUPAC) and A Guide to IUPAC Nomenclature of Organic Compounds (Recommendations 1993), (Blackwell Scientific Publications, 1993, Copyright 1993 IUPAC); or commercially available software packages such as Autonom (brand of nomenclature software provided in the ChemDraw Ultra® office suite marketed by CambridgeSoft.com); and ACD/Index Name™ (brand of commercial nomenclature software marketed by Advanced Chemistry Development, Inc., Toronto, Ontario).

Abbreviations

As used herein, the following abbreviations are intended to have the following meanings (additional abbreviations are provided where needed throughout the Specification):

Boc tert-butoxycarbonyl

DCM dichloromethane

DMF dimethylformamide

DMSO dimethylsulfoxide

DIEA diisopropylethylamine

EDTA ethylenediaminetetraaceticacid

EDC 1-(3-dimethylaminopropyl)-3-ethylcarbodiimide hydrochloride

EtOAc ethyl acetate

HOBT 1-hydroxybenzotriazole hydrate

HBTU O-benzotriazol- 1-yl-N,N,N′,N′-tetramethyluronium hexafluorophosphate

i-PrOH isopropyl alcohol

LC/MS (ESI) Liquid chromatography/mass spectrum (electrospray ionization)

MeOH Methyl alcohol

NMM N-methylmorpholine

NMR nuclear magnetic resonance

PS polystyrene

RT room temperature

NaHMDS sodium hexamethyldisilazane

TEA triethylamine

TFA trifluoroacetic acid

THF tetrahydrofuran

TLC thin layer chromatography

Formula I and Formula II

The present invention comprises a compound selected from: the group consisting of Formula I and Formula II:

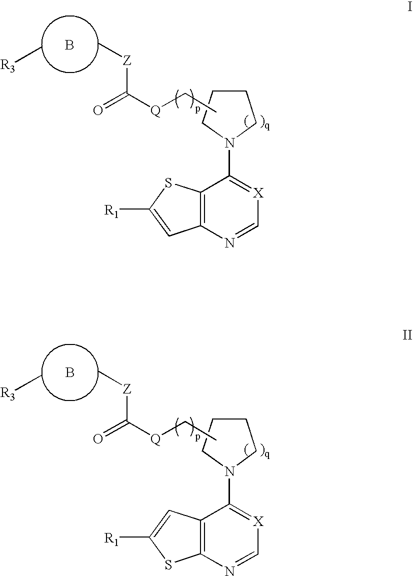

and N-oxides, pharmaceutically acceptable salts, and stereochemical isomers thereof, wherein:

q is 0, 1 or 2;

p is 0 or 1;

Q is NH, N(alkyl), O,l or a direct bond;

X is N or CH;

Z is NH, N(alkyl), or CH₂;

B is aryl (wherein said aryl is preferably phenyl), cyclopentadienyl, cycloalkyl (wherein said cycloalkyl is preferably cyclopentanyl, cyclohexanyl, cyclopentenyl or cyclohexenyl), heteroaryl (wherein said heteroaryl is preferably pyrrolyl, furanyl, thiophenyl, imidazolyl, thiazolyl, oxazolyl, pyranyl, thiopyranyl, pyridinyl, pyrimidinyl, pyrazinyl, pyridinyl-N-oxide, or pyrrolyl-N-oxide, and most preferably pyrrolyl, furanyl, thiophenyl, imidazolyl, thiazolyl, oxazolyl, pyridinyl, pyrimidinyl, or pyrazinyl), or a nine to ten membered benzo-fused heteroaryl (wherein said nine to ten membered benzo-fused heteroaryl is preferably benzothiazolyl, benzooxazolyl, benzoimidazolyl, benzofuranyl, indolyl, quinolinyl, isoquinolinyl, or benzo [b]thiophenyl);

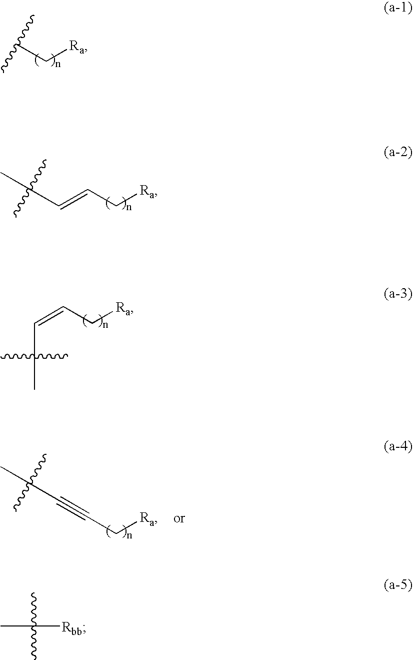

wherein n is 1, 2, 3 or 4;
Rₐ is hydrogen, heteroaryl optionally substituted with R₅ (wherein said heteroaryl is preferably pyrrolyl, furanyl, thiophenyl, imidazolyl, thiazolyl, oxazolyl, pyranyl, thiopyranyl, pyridinyl, pyrimidinyl, triazolyl, pyrazinyl, pyridinyl-N-oxide, or pyrrolyl-N-oxide, and most preferably pyrrolyl, furanyl, thiophenyl, imidazolyl, thiazolyl, oxazolyl, pyridinyl, pyrimidinyl, triazolyl, or pyrazinyl), hydroxyl, alkylamino, dialkylamino, oxazolidinonyl optionally substituted with R₅, pyrrolidinonyl optionally substituted with R₅, piperidinonyl optionally substituted with R₅, cyclic heterodionyl optionally substituted with R₅, heterocyclyl optionally substituted with R₅ (wherein said heterocyclyl is preferably pyrrolidinyl, tetrahydrofuranyl, tetrahydrothiophenyl, imidazolidinyl, thiazolidinyl, oxazolidinyl, tetrahydropyranyl, tetrahydrothiopyranyl, thiomorphlinyl, thiomorpholinyl-1, 1-dioxide, piperidinyl, morpholinyl or piperazinyl), —COOR_{y}, —CONR_{w}Rₓ, —N(R_{y})CON(R_{w})(Rₓ), —N(R_{w})C(O)ORₓ, —N(R_{w})COR_{y}, —SR_{y}, —SOR_{y}, —SO₂R_{y}, —NR_{w}SO₂R_{y}, —NR_{w}SO₂Rₓ, —SO₃R_{y}, or —OSO₂NR_{w}Rₓ;
R_{bb} is hydrogen, halogen, aryl, heteroaryl, or heterocyclyl;
R₅ is one, two, or three substituents independently selected from: halogen, cyano, trifluoromethyl, amino, hydroxyl, alkoxy, —C(O)alkyl, —SO₂alkyl, —C(O)N(alkyl)₂, alkyl, —C(₁₋₄)alkyl-OH, or alkylamino;

R_{w} and Rₓ are independently selected from: hydrogen, alkyl, alkenyl, aralkyl (wherein the aryl portion of said aralkyl is preferrably phenyl), or heteroaralkyl (wherein the heteroaryl portion of said heteroaralkyl is preferably pyrrolyl, furanyl, thiophenyl, imidazolyl, thiazolyl, oxazolyl, pyranyl, thiopyranyl, pyridinyl, pyrimidinyl, pyrazinyl, pyridinyl-N-oxide, or pyrrolyl-N-oxide, and most preferably pyrrolyl, furanyl, thiophenyl, imidazolyl, thiazolyl, oxazolyl, pyridinyl, pyrimidinyl, or pyrazinyl), or R_{w} and Rₓ may optionally be taken together to form a 5 to 7 membered ring, optionally containing a heteromoiety selected from: O, NH, N(alkyl), SO₂, SO, or S, preferably selected from the group consisting of: 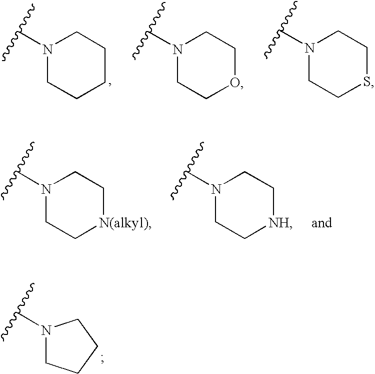
R_{y} is selected from: hydrogen, alkyl, alkenyl, cycloalkyl (wherein said cycloalkyl is preferably cyclopentanyl or cyclohexanyl), aryl (wherein said aryl is preferably phenyl), aralkyl (wherein the aryl portion of said aralkyl is preferably phenyl), heteroaralkyl (wherein the heteroaryl portion of said heteroaralkyl is preferably pyrrolyl, furanyl, thiophenyl, imidazolyl, thiazolyl, oxazolyl, pyranyl, thiopyranyl, pyridinyl, pyrimidinyl, pyrazinyl, pyridinyl-N-oxide, or pyrrolyl-N-oxide, and most preferably pyrrolyl, furanyl, thiophenyl, imidazolyl, thiazolyl, oxazolyl, pyridinyl, pyrimidinyl, or pyrazinyl), or heteroaryl (wherein said heteroaryl is preferably pyrrolyl, furanyl, thiophenyl, imidazolyl, thiazolyl, oxazolyl, pyranyl, thiopyranyl, pyridinyl, pyrimidinyl, pyrazinyl, pyridinyl-N-oxide, or pyrrolyl-N-oxide, and most preferably pyrrolyl, furanyl, thiophenyl, imidazolyl, thiazolyl, oxazolyl, pyridinyl, pyrimidinyl, or pyrazinyl); and

R₃ is one or more substituents, optionally present, and independently selected from: alkyl, alkoxy, halogen, alkoxyether, hydroxyl, thio, nitro, cycloalkyl optionally substituted with R4 (wherein said cycloalkyl is preferably cyclopentanyl or cyclohexanyl), heteroaryl optionally substituted with R4 (wherein said heteroaryl is preferably pyrrolyl, furanyl, thiophenyl, imidazolyl, thiazolyl, oxazolyl, pyranyl, thiopyranyl, pyridinyl, pyrimidinyl, pyrazinyl, pyridinyl-N-oxide, or pyrrolyl-N-oxide; and most preferably pyrrolyl, furanyl, thiophenyl, imidazolyl, thiazolyl, oxazolyl, pyridinyl, pyrimidinyl, or pyrazinyl), alkylamino, heterocyclyl optionally substituted with R4 (wherein said heterocyclyl is preferably azapenyl, pyrrolidinyl, tetrahydrofuranyl, tetrahydrothiophenyl, imidazolidinyl, thiazolidinyl, oxazolidinyl, tetrahydropyranyl, tetrahydrothiopyranyl, piperidinyl, morpholinyl, or piperazinyl), partially unsaturated heterocyclyl optionally substituted with R₄, (wherein said partially unsaturated heterocyclyl is preferably tetrahydropyridinyl. tetrahydropyrazinyl, dihydrofuranyl, dihydrooxazinyl, dihydropyrrolyl, or dihydroimidazolyl), -O(cycloalkyl), pyrrolidinone optionally substituted with R₄, phenoxy optionally substituted with R₄, —CN, —OCHF₂, —OCF₃, —CF₃, halogenated alkyl, heteroaryloxy optionally substituted with R₄, dialkylamino, —NHSO₂alkyl, thioalkyl, or —SO₂alkyl; wherein R4 is independently selected from: halogen, cyano, trifluoromethyl, amino, hydroxyl, alkoxy, —C(O)alkyl, —CO₂alkyl, —SO₂alkyl, —C(O)N(alkyl)₂, alkyl, or alkylamino.

As used hereafter, the term “compounds of Formula I”, “compounds of Formula II”and “Compounds of Formula I and Formula II” are meant to include also the N-oxides, pharmaceutically acceptable salts, and stereochemical isomers thereof.

Embodiments of Formula I and Formula II

In an embodiment of the present invention: N-oxides are optionally present on one or more of: N-1 or N-3 (when X is N) (see FIG. 1 below for ring numbers).
FIG. 1

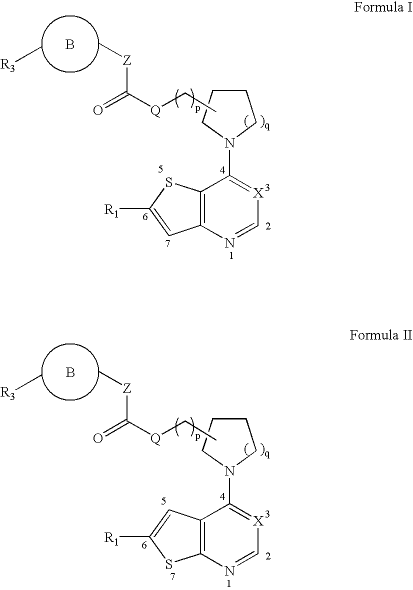

FIG. 1 illustrates ring atoms numbered 1 through 7, as used in the present specification.

Preferred embodiments of the invention are compounds of Formula I and Formula II wherein one or more of the following limitations are present:

q is 1 or 2;

p is 0 or 1;

Q is NH, N(alkyl), O, or a direct bond;

X is N;

Z is NH, N(alkyl), or CH₂;

B is aryl or heteroaryl;

R₁ is: 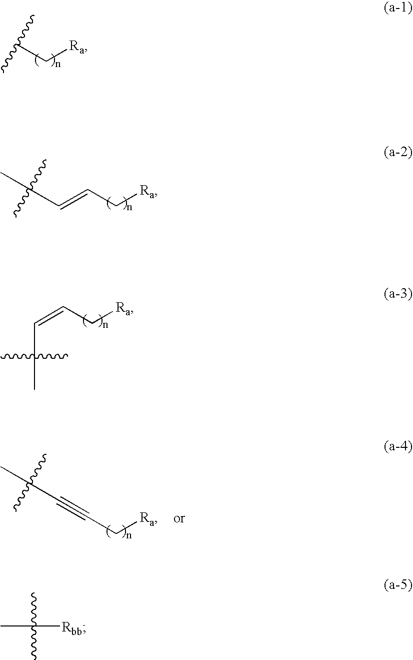
wherein n is 1, 2, 3 or 4;
Rₐ is hydrogen, heteroaryl optionally substituted with R₅, hydroxyl, alkylamino, dialkylamino, oxazolidinonyl optionally substituted with R₅, pyrrolidinonyl optionally substituted with R₅, piperidinonyl optionally substituted with R₅, cyclic heterodionyl optionally substituted with R₅, heterocyclyl optionally substituted with R₅, —COOR_{y}, —CONR_{w}Rₓ, —N(R_{y})CON(R_{w})(Rₓ), —N(R_{w})C(O)ORₓ, —N(R_{w})COR_{y}, —SR_{y}, —SOR_{y}, —SO₂R_{y}, —NR_{w}SO₂R_{y}, —NR_{w}SO₂Rₓ, —SO₃R_{y}, or —OSO₂NR_{w}Rₓ;
R_{bb} is hydrogen, halogen, aryl, heteroaryl, or heterocyclyl;
R₅ is one, two, or three substituents independently selected from: halogen, cyano, trifluoromethyl, amino, hydroxyl, alkoxy, —C(O)alkyl, —SO₂alkyl, —C(O)N(alkyl)₂, alkyl, —C(₁₋₄)alkyl-OH, or alkylamino;
R_{w} and Rₓ are independently selected from: hydrogen, alkyl, alkenyl, aralkyl, or heteroaralkyl, or R_{w} and Rₓ may optionally be taken together to form a 5 to 7 membered ring, optionally containing a heteromoiety selected from O, NH, N(alkyl), SO₂, SO, or S;
R_{y} is selected from: hydrogen, alkyl, alkenyl, cycloalkyl, aryl, aralkyl, heteroaralkyl, or heteroaryl; and

R₃ is one or more substituents, optionally present, and independently selected from: alkyl, alkoxy, halogen, alkoxyether, hydroxyl, thio, nitro, cycloalkyl optionally substituted with R₄, heteroaryl optionally substituted with R₄, alkylamino, heterocyclyl optionally substituted with R₄, partially unsaturated heterocyclyl optionally substituted with R₄, -O(cycloalkyl), pyrrolidinone optionally substituted with R₄, phenoxy optionally substituted with R₄, —CN, —OCHF₂, —OCF₃, —CF₃, halogenated alkyl, heteroaryloxy optionally substituted with R₄, dialkylamino, —NHSO₂alkyl, thioalkyl, or —SO₂alkyl; wherein R4 is independently selected from: halogen, cyano, trifluoromethyl, amino, hydroxyl, alkoxy, —C(O)alkyl, —CO₂alkyl, —SO₂alkyl, —C(O)N(alkyl)₂, alkyl, or alkylamino.

Other preferred embodiments of the invention are compounds of Formula I and Formula II wherein one or more of the following limitations are present:

q is 1 or 2;

p is 0 or 1;

Q is NH, O, or a direct bond;

X is N;

Z is NH or CH₂;

B is aryl or heteroaryl;

R₁ is: 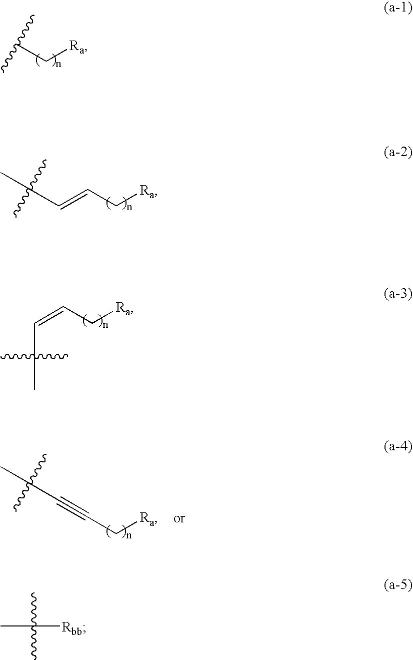
wherein n is 1, 2, 3 or 4;
Rₐ is hydrogen, heteroaryl optionally substituted with R₅, hydroxyl, alkylamino, dialkylamino, oxazolidinonyl optionally substituted with R₅, pyrrolidinonyl optionally substituted with R₅, piperidinonyl optionally substituted with R₅, cyclic heterodionyl optionally substituted with R₅, heterocyclyl optionally substituted with R₅, —COOR_{y}, —CONR_{w}Rₓ, —N(R_{y})CON(R_{w})(Rₓ), —N(R_{w})C(O)ORₓ, —N(R_{w})COR_{y}, —SR_{y}, —SOR_{y}, —SO₂R_{y}, —NR_{w}SO₂R_{y}, —NR_{w}SO₂Rₓ, —SO₃R_{y}, or —OSO₂NRRₓ;
R_{bb} is hydrogen, halogen, aryl, heteroaryl, or heterocyclyl;
R₅ is one, two, or three substituents independently selected from: halogen, cyano, trifluoromethyl, amino, hydroxyl, alkoxy, —C(O)alkyl, —SO₂alkyl, —C(O)N(alkyl)₂, alkyl, —C₍₁₋₄₎alkyl-OH, or alkylamino;
R_{w} and Rₓ are independently selected from: hydrogen, alkyl, alkenyl, aralkyl, or heteroaralkyl, or R_{w} and Rₓ may optionally be taken together to form a 5 to 7 membered ring, optionally containing a heteromoiety selected from O, NH, N(alkyl), SO₂, SO, or S;
R_{y} is selected from: hydrogen, alkyl, alkenyl, cycloalkyl, aryl, aralkyl, heteroaralkyl, or heteroaryl; and

R₃ is one or more substituents, optionally present, and independently selected from: alkyl, alkoxy, halogen, alkoxyether, cycloalkyl optionally substituted with R₄, alkylamino, heterocyclyl optionally substituted with R₄, -O(cycloalkyl), phenoxy optionally substituted with R₄, dialkylamino, or —SO₂alkyl; wherein R₄ is independently selected from: halogen, cyano, trifluoromethyl, amino, hydroxyl, alkoxy, —C(O)alkyl, —CO₂alkyl, —SO₂alkyl, —C(O)N(alkyl)₂, alkyl, or alkylamino.

Still other preferred embodiments of the invention are compounds of Formula I and Formula II wherein one or more of the following limitations are present:

q is 1 or 2;

p is 0 or 1;

Q is NH, O, or a direct bond;

Z is NH or CH₂;

B is aryl or heteroaryl;

X is N;

R₁is: 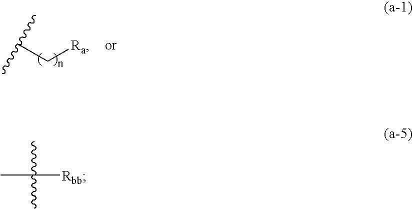
wherein n is 1, 2, 3 or 4;
Rₐ is hydrogen, hydroxyl, alkylamino, dialkylamino, heterocyclyl optionally substituted with R₅, —CONR_{w}Rₓ, —N(R_{y})CON(R_{w})(Rₓ), —N(R_{w})C(O)ORₓ, —N(R_{w})COR_{y}, —SO₂R_{y}, —NR_{w}SO₂R_{y}, or —NR_{w}SO₂Rₓ;
R_{bb} is hydrogen, halogen, aryl, heteroaryl, or heterocyclyl;
R₅ is one, two, or three substituents independently selected from: halogen, cyano, trifluoromethyl, amino, hydroxyl, alkoxy, —C(O)alkyl, —SO₂alkyl, —C(O)N(alkyl)₂, alkyl, —C₍₁₋₄₎alkyl-OH, or alkylamino;
R_{w} and Rₓ are independently selected from: hydrogen, alkyl, alkenyl, aralkyl, or heteroaralkyl, or R_{w} and Rₓ may optionally be taken together to form a 5 to 7 membered ring, optionally containing a heteromoiety selected from O, NH, N(alkyl), SO₂, SO, or S;

R_{y} is selected from: hydrogen, alkyl, alkenyl, cycloalkyl, aryl, aralkyl, heteroaralkyl, or heteroaryl; and

R₃ is one substituentselected from: alkyl, alkoxy, halogen, alkoxyether, cycloalkyl optionally substituted with R₄, alkylamino, heterocyclyl optionally substituted with R₄, -O(cycloalkyl), phenoxy optionally substituted with R₄, dialkylamino, or —SO₂alkyl; wherein R₄ is independently selected from: halogen, cyano, trifluoromethyl, amino, hydroxyl, alkoxy, —C(O)alkyl, —CO₂alkyl, —SO₂alkyl, —C(O)N(alkyl)₂, alkyl, or alkylamino.

Particularly preferred embodiments of the invention are compounds of Formula I and Formula II wherein one or more of the following limitations are present:

q is 1 or 2;

p is 0 or 1;

Q is NH, O, or a direct bond;

Z is NH or CH₂;

B is phenyl or pyridyl;

X is N;

R₁ is: 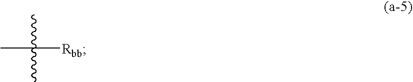
wherein
R_{bb} is hydrogen, halogen, aryl, or heteroaryl;
and

R₃ is one substituent selected from: alkyl, alkoxy, heterocyclyl, -O(cycloalkyl), phenoxy, or dialkylamino.

Most particularly preferred embodiments of the invention are compounds of Formula I and Formula II wherein one or more of the following limitations are present:

q is 1 or 2;

p is 0;

Q is NH or O;

Z is NH;

B is phenyl or pyridyl;

X is N;

R₁ is: 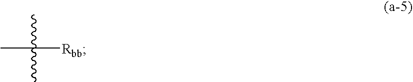
wherein
R_{bb} is hydrogen;
and

R₃ is one substituent selected from: alkyl, —O(cycloalkyl), phenoxy, or dialkylamino.

Pharmaceeutically Acceptably Salts

The compounds of the present invention may also be present in the form of pharmaceutically acceptable salts.

For use in medicines, the salts of the compounds of this invention refer to non-toxic “pharmaceutically acceptable salts.” FDA approved pharmaceutically acceptable salt forms (Ref. International J. Pharm. 1986, 33, 201-217; J. Pharm. Sci., 1977, January 66(1), p 1) include pharmaceutically acceptable acidic/anionic or basic/cationic salts.

Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate (SEH), sodium hydroxide, triethanolamine (TEA) or zinc.

Prodrugs

The present invention includes within its scope prodrugs of the compounds of the invention. In general, such prodrugs will be functional derivatives of the compounds which are readily convertible in vivo into an active compound. Thus, in the methods of treatment of the present invention, the term “administering” shall encompass the means for treating, ameliorating or preventing a syndrome, disorder or disease described herein with a compound specifically disclosed or a compound, or prodrug thereof, which would obviously be included within the scope of the invention albeit not specifically disclosed for certain of the instant compounds. Conventional procedures for the selection and preparation of suitable prodrug derivatives are described in, for example, “Design of Prodrugs”, ed. H. Bundgaard, Elsevier, 1985.

Stereochemical Isomers

One skilled in the art will recognize that the compounds of Formula I and Formula II may have one or more asymmetric carbon atoms in their structure. It is intended that the present invention include within its scope single enantiomer forms of the compounds, racemic mixtures, and mixtures of enantiomers in which an enantiomeric excess is present.

The term “single enantiomer” as used herein defines all the possible homochiral forms which the compounds of Formula I and Formula II and their N-oxides, addition salts, quaternary amines or physiologically functional derivatives may possess. Stereochemically pure isomeric forms may be obtained by the application of art known principles. Diastereoisomers may be separated by physical separation methods such as fractional crystallization and chromatographic techniques, and enantiomers may be separated from each other by the selective crystallization of the diastereomeric salts with optically active acids or bases or by chiral chromatography. Pure stereoisomers may also be prepared synthetically from appropriate stereochemically pure starting materials, or by using stereoselective reactions.

The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (enantiomers).

The term “stereoisomer” refers to isomers of identical constitution that differ in the arrangement of their atoms in space. Enantiomers and diastereomers are examples of stereoisomers.

The term “chiral” refers to the structural characteristic of a molecule that makes it impossible to superimpose it on its mirror image.

The term “enantiomer” refers to one of a pair of molecular species that are mirror images of each other and are not superimposable.

The term “diastereomer” refers to stereoisomers that are not mirror images.

The symbols “R” and “S” represent the configuration of substituents around a chiral carbon atom(s).

The term “racemate” or “racemic mixture” refers to a composition composed of equimolar quantities of two enantiomeric species, wherein the composition is devoid of optical activity.

The term “homochiral” refers to a state of enantiomeric purity.

The term “optical activity” refers to the degree to which a homochiral molecule or nonracemic mixture of chiral molecules rotates a plane of polarized light.

The term “geometric isomer” refers to isomers that differ in the orientation of substituent atoms in relationship to a carbon-carbon double bond, to a cycloalkyl ring or to a bridged bicyclic system. Substituent atoms (other than H) on each side of a carbon-carbon double bond may be in an E or Z configuration. In the “E” (opposite sided) configuration, the substituents are on opposite sides in relationship to the carbon-carbon double bond; in the “Z” (same sided) configuration, the substituents are oriented on the same side in relationship to the carbon-carbon double bond. Substituent atoms (other than hydrogen) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. Compounds having a mixture of “cis” and “trans” species are designated “cis/trans”.

It is to be understood that the various substituent stereoisomers, geometric isomers and mixtures thereof used to prepare compounds of the present invention are either commercially available, can be prepared synthetically from commercially available starting materials or can be prepared as isomeric mixtures and then obtained as resolved isomers using techniques well-known to those of ordinary skill in the art.

The isomeric descriptors “R,” “S,” “E,” “Z,” “cis,” and “trans” are used as described herein for indicating atom configuration(s) relative to a core molecule and are intended to be used as defined in the literature (IUPAC Recommendations for Fundamental Stereochemistry (Section E), Pure Appl. Chem., 1976, 45:13-30). The compounds of the present invention may be prepared as individual isomers by either isomer-specific synthesis or resolved from an isomeric mixture. Conventional resolution techniques include forming the free base of each isomer of an isomeric pair using an optically active salt (followed by fractional crystallization and regeneration of the free base), forming an ester or amide of each of the isomers of an isomeric pair (followed by chromatographic separation and removal of the chiral auxiliary) or resolving an isomeric mixture of either a starting material or a final product using preparative TLC (thin layer chromatography) or a chiral HPLC column.

Polymorphs

Furthermore, compounds of the present invention may have one or more polymorph or amorphous crystalline forms and as such are intended to be included in the scope of the invention. In addition, some of the compounds may form solvates with water (i.e., hydrates) or common organic solvents, and such are also intended to be encompassed within the scope of this invention.

N-Oxides

The compounds of Formula I and Formula II may be converted to the corresponding N-oxide forms following art-known procedures for converting a trivalent nitrogen into its N-oxide form. Said N-oxidation reaction may generally be carried out by reacting the starting material of Formula I (or Formula II) with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. tbutyl hydro-peroxide. Suitable solvents are, for example, water, lower alcohols, e.g. ethanol and the like, hydrocarbons, e.g. toluene, ketones, e.g. 2-butanone, halogenated hydrocarbons, e.g. dichloromethane, and mixtures of such solvents.

Tautomeric Forms

Some of the compounds of Formula I and Formula II may also exist in their tautomeric forms. Such forms although not explicitly indicated in the present application are intended to be included within the scope of the present invention.

Preparation of Compounds of the Present Invention

During any of the processes for preparation of the compounds of the present invention, it may be necessary and/or desirable to protect sensitive or reactive groups on any of the molecules concerned. This may be achieved by means of conventional protecting groups, such as those described in Protecting Groups, P. Kocienski, Thieme Medical Publishers, 2000; and T. W. Greene & P. G. M. Wuts, Protective Groups in Organic Synthesis, 3^{rd} ed. Wiley Interscience, 1999. The protecting groups may be removed at a convenient subsequent stage using methods known in the art.

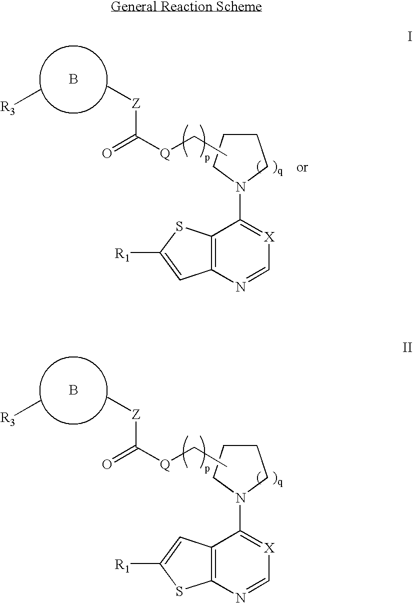

Compounds of Formulae I or II can be prepared by methods known to those who are skilled in the art. The following reaction schemes are only meant to represent examples of the invention and are in no way meant to be a limit of the invention.

The compounds of Formula I, wherein Q is O and p, q, B, X, Z, R₁ and R₃ are as defined in Formula I, may be synthesized as outlined by the general synthetic route illustrated in Scheme 1. Treatment of an appropriate 4-chloro-thieno[3,2-d]pyrimidine or pyridine III with an appropriate hydroxy cyclic amine IV in a solvent such as isopropanol at a temperature of 50° C. to 150° C. can provide the intermediate V. Treatment of intermediate V with a base such as sodium hydride in a solvent such as tetrahydrofuran (THF) followed by addition of the appropriate acylating group VI, wherein LG is an appropriate leaving group, such as chloride, p-nitrophenoxy or imidazole, can provide the final product I. The 4-chloro-thieno[3,2-d]pyrimidines or pyridines III are either commercially available or can be prepared by known methods (WO9924440); the hydroxy cyclic amines IV are commercially available or can be derived by known methods (JOC, 1961, 26, 1519; EP314362). The acylating reagents VI are either commercially available or can be prepared as illustrated in Scheme 1. Treatment of an appropriate R₃BZH, wherein Z is NH or N(alkyl), with an appropriate acylating reagent such as carbonyldiimidazole or p-nitrophenylchloroformate in the presence of a base such as triethylamine can provide VI. Many R₃BZH reagents are commercially available or can be prepared by a number of known methods (e.g. Tet Lett 1995, 36, 2411-2414). Corrresponding compounds of Formula II can be prepared by the same method outlined in Scheme 1 using the appropriate 4-chloro-thieno[2,3-d]pyrimidine or pyridine.

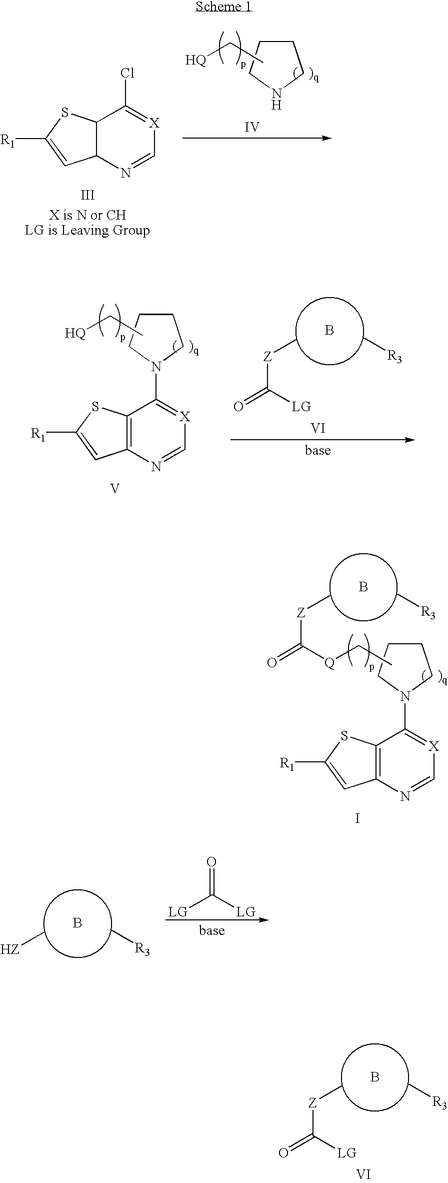

Alternatively compounds of Formula I, wherein Q is O, Z is NH or N(alkyl), and p, q, B, X, R₁ and R₃ are as defined in Formula I, may be synthesized as outlined by the general synthetic route illustrated in Scheme 2. Treatment of alcohol intermediate V, prepared as described in Scheme 1, with an acylating agent such as carbonyldiimidazole or p-nitrophenylchloroformate, wherein LG may be chloride, p-nitrophenoxy or imidazole, can provide the acylated intermediate VII. Treatment of VII with an appropriate R₃BZH, wherein Z is NH or N(alkyl), can provide the final product I. Corresponding compounds of Formula II can be prepared by the same method outlined in Scheme 2 using the appropriate 4-chloro-thieno[2,3-d]pyrimidine or pyridine.

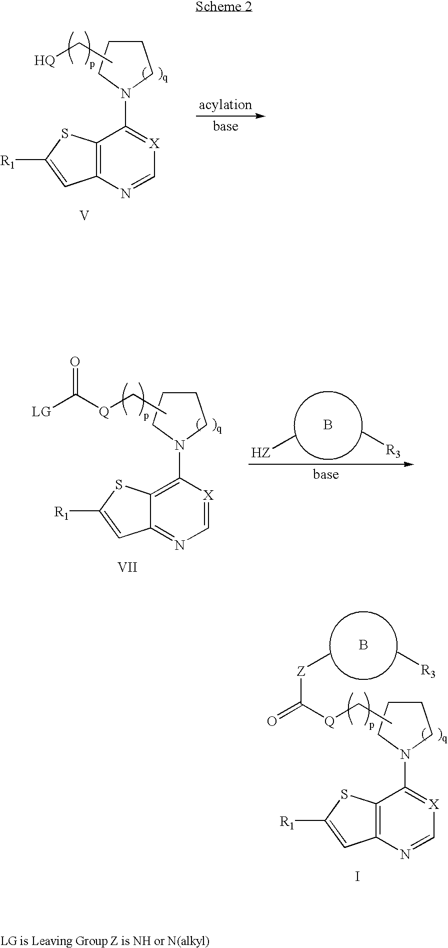

An alternative method to prepare compounds of Formula I, wherein Q is O, Z is NH, and p, q, B, X, R₁ and R₃ are as defined in Formula I, is illustrated in Scheme 3. Treatment of alcohol intermediate V, prepared as described in Scheme 1, with an appropriate isocyanate in the presence of a base such as triethylamine can provide the final product I. The isocyanates are either commercially available or can be prepared by a known method (J. Org Chem, 1985, 50, 5879-5881). Corresponding compounds of Formula II can be prepared by the same method outlined in Scheme 3 using the appropriate 4-chloro-thieno[2,3-d]pyrimidine or pyridine.

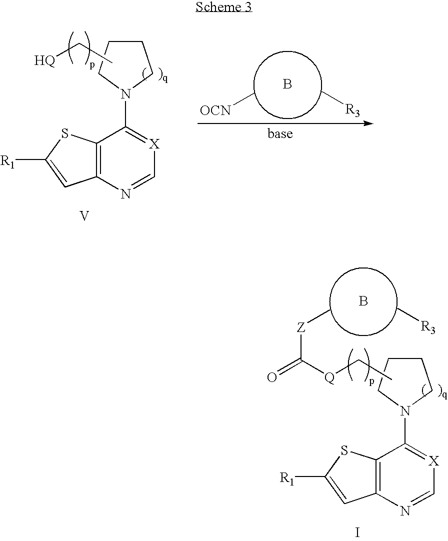

A method for preparing compounds of Formula I, wherein Q is NH or N(alkyl), and p, q, B, X, Z, R₁ and R₃ are as defined in Formula I, is outlined by the general synthetic route illustrated in Scheme 4. Treatment of the appropriate 4-chloro-thieno[3,2-d]pyrimidine or pyridine III with an N-protected aminocyclic amine VIII, where PG is an amino protecting group known to those skilled in the art, in a solvent such as isopropanol at a temperature of 50° C. to 150° C. can provide intermediate IX. Deprotection of the amino protecting group (PG) under standard conditions known in the art can provide compound X. Acylation of X in the presence of a base such as diisopropylethylamine with an appropriate reagent VI, wherein Z is NH or N(alkyl) and LG may be chloride, p-nitrophenoxy, or imidazole, or, when Z is CH₂, via coupling with an appropriate R₃BCH₂CO₂H using a standard coupling reagent such as 1-(3-dimethylaminopropyl)-3-ethylcarbodiimide hydrochloride (EDC) or 1-hydroxybenzotriazole (HOBT), can provide the final product I. The amino cyclic amines are commercially available or are derived by known methods (U.S. Pat. No. 4,822,895; EP401623). The acylating reagents VI are either commercially available or can be prepared as outlined in Scheme 1. Additionally, compounds of Formula I, wherein Z is NH, can be obtained by treatment of intermediate X with an appropriate isocyanate. The isocyanates are either commercially available or can be prepared by a known method (J. Org Chem, 1985, 50, 5879-5881). Corresponding compounds of Formula II can be prepared by the same method outlined in Scheme 4 using the appropriate 4-chloro-thieno[2,3-d]pyrimidine or pyridine.

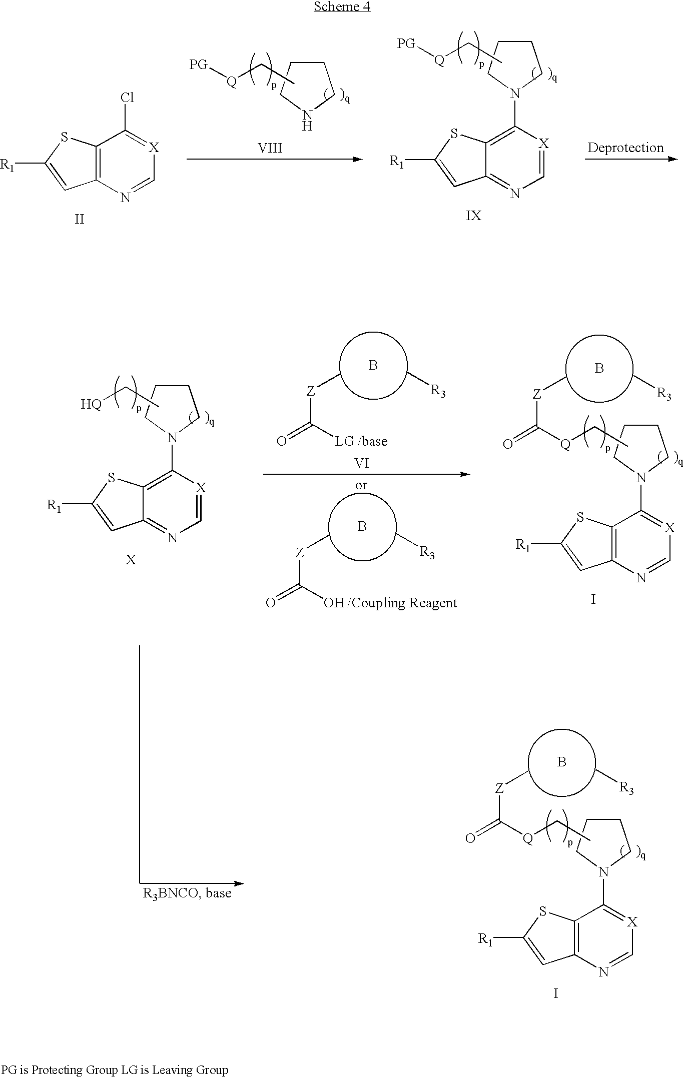

A method for preparing compounds of Formula I, wherein Q is a direct bond, Z is NH or N(alkyl), and p, q, B, X, R₁ and R₃ are as defined in Formula I, is outlined by the general synthetic route illustrated in Scheme 5. Reacting the appropriate 4-chloro-thieno[3,2-d]pyrimidine or pyridine III with a cyclic aminoester XI in a solvent such as isopropanol at a temperature of 50° C. to 150° C. followed by basic hydrolysis of the ester functionality can provide intermediate XII. Coupling of an appropriate R₃BZH, wherein Z is NH or N(alkyl), to XII using a standard coupling reagent such as 1-(3-dimethylaminopropyl)-3-ethylcarbodiimide hydrochloride (EDC) or carbonyldiimidazole can provide final compound I. Corresponding compounds of Formula II can be prepared by the same method outlined in Scheme 5 using the appropriate 4-chloro-thieno[2,3-d]pyrimidine or pyridine.

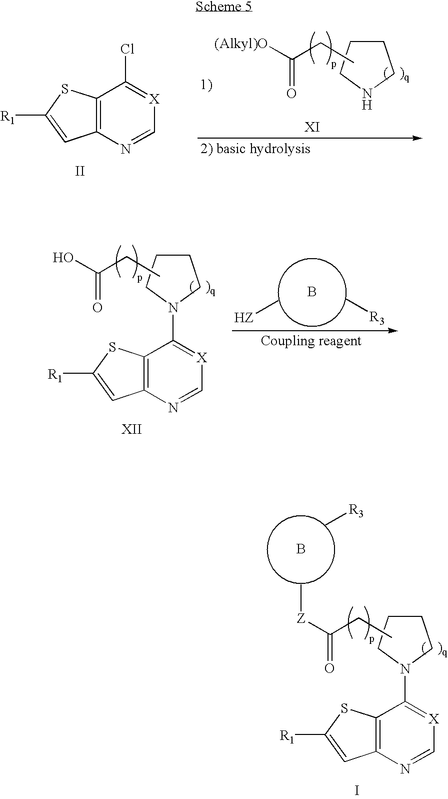

Compounds of Formula I wherein R₁ is Rbb, and R_{bb} is aryl or heteroaryl, and Q, p, q, B, X, Z, and R₃ are as defined in Formula I, can also be prepared as outlined in Scheme 6. Preparation of the appropriate bromothienopyrimidine/bromothienopyridine XIV can be derived from the known 6-bromo-4-chloro-thieno[3,2-d]pyrimidine or pyridine XIII (W09924440) utilizing the reaction sequences outlined in schemes 1-5, in which XIII is used in place of II. Treatment of bromide XIV with an appropriate aryl boronic acid or aryl boronic ester, wherein R is H or alkyl, in the presence of a palladium catalyst such as bis(triphenylphosphine)palladium dichloride in a solvent such as toluene at a temperature of 50° C. to 200° C. can provide the final product I. The boronic acids/boronic esters are either commercially available or prepared by known methods (Synthesis 2003, 4, 469-483; Organic letters 2001, 3, 1435-1437). Corresponding compounds of Formula II can be prepared by the same method outlined in Scheme 6 using the appropriate 6-bromo-4-chloro-thieno[2,3-d]pyrimidine or pyridine.

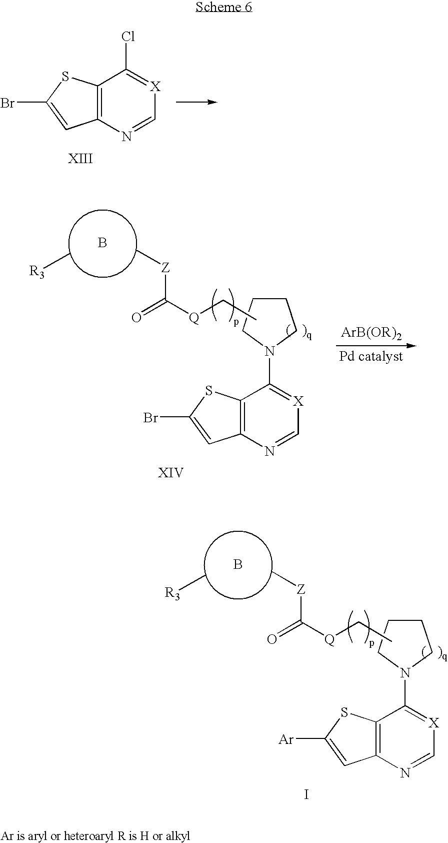

Compounds of Formula I, wherein R₁ is —CHCH(CH₂)nRₐ and Q, p, q, B, X, Z, and R₃ are as defined in Formula I, can also be prepared as outlined in Scheme 7. Preparation of the appropriate bromothienopyrimidine/bromothienopyridine XIV can be derived from the known 6-bromo-4-chloro-thieno[3,2-d]pyrimidine or pyridine XIII (W09924440) utilizing the reaction sequences outlined in schemes 1-5, in which XIII is used in place of II. Treatment of XIV with an appropriate vinylstannane XV in the presence of a palladium catalyst such as bis(triphenylphosphine)palladium dichloride and a solvent such as dimethylformamide at a temperature of 25° C. to 150° C. can provide the alkenyl alcohol XVI. Conversion of the alcohol XVI to an appropriate leaving group known by those skilled in the art such as a mesylate, followed by an SN₂ displacement reaction of XVII with an appropriate nucleophilic heterocycle, heteroaryl, amine, alcohol, sulfonamide, or thiol can provide the final compound I. The corresponding cis olefin isomers of Formula I can be prepared by the same method utilizing the appropriate cis vinyl stannane. If Rₐ nucleophile is a thiol, further oxidation of the thiol can provide the corresponding sulfoxides and sulfones. If Rₐ nucleophile is an amino, acylation of the nitrogen with an appropriate acylating or sulfonylating agent can provide the corresponding amides, carbamates, ureas, and sulfonamides. If the desired Rₐ is COOR_{y} or CONR_{w}Rₓ, these can be derived from the corresponding hydroxyl group. Oxidation of the hydroxyl group to the acid followed by ester or amide formation under conditions known in the art can provide examples wherein Rₐ is COOR_{y} or CONR_{w}Rₓ. Compounds of Formula I that have R₁ as a (CH₂)ₙRₐ can be derived from the corresponding alkene I by reduction of the olefin under conditions known in the art. Corresponding compounds of Formula II can be prepared by the same method outlined in Scheme 7 using the appropriate 6-bromo-4-chloro-thieno[2,3-d]pyrimidine or pyridine.

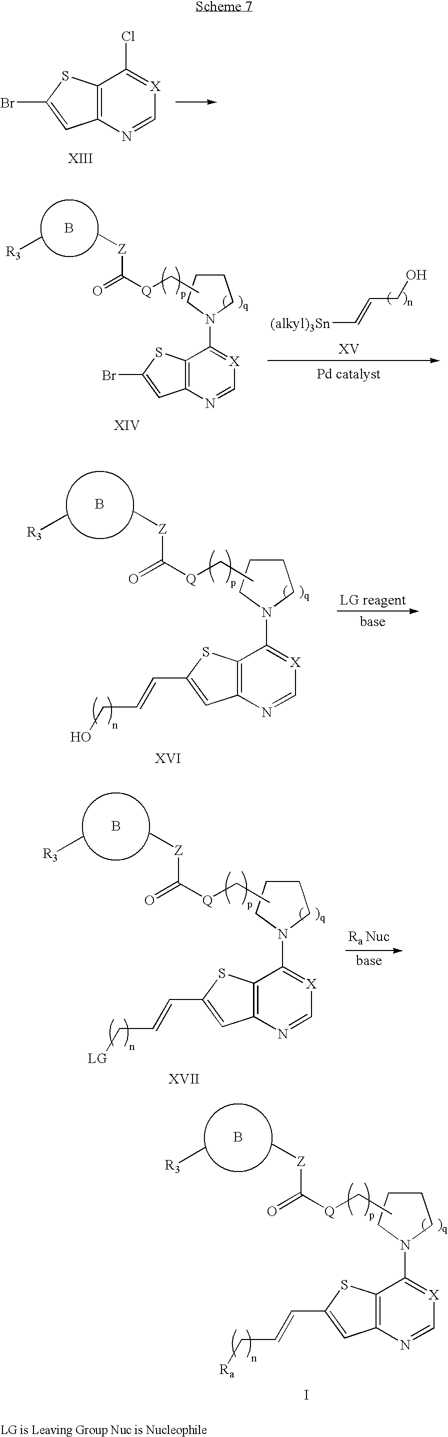

Compounds of Formula I, wherein R₁ is —CC(CH₂)ₓRₐ and Q, p, q, B, X, Z, and R₃ are as defined in Formula I, can also be prepared as outlined in Scheme 8. Preparation of the appropriate bromothienopyrimidine/bromothienopyridine XIV can be derived from the known 6-bromo-4-chloro-thieno[3,2-d]pyrimidine or pyridine XIII (WO9924440) utilizing the reaction sequences outlined in schemes 1-5, in which XIII is used in place of II. Treatment of XIV with an appropriate alkynyl alcohol in the presence of a palladium catalyst such as bis(triphenylphosphine)palladium dichloride, a copper catalyst such as copper(I) iodide, a base such as diethylamine and a solvent such as dimethylformamide at a temperature of 25° C. to 150° C. can provide the alkynyl alcohol XVIII. Conversion of the alcohol XVIII to an appropriate leaving group known by those skilled in the art such as a mesylate, followed by an SN₂ displacement reaction of XIX with an appropriate nucleophilic heterocycle, heteroaryl, amine, alcohol, sulfonamide, or thiol can provide the final compound I. If Rₐ nucleophile is a thiol, further oxidation of the thiol can provide the corresponding sulfoxides and sulfones. If Rₐ nucleophile is an amino, acylation of the nitrogen with an appropriate acylating or sulfonylating agent can provide the corresponding amides, carbamates, ureas, and sulfonamides. If the desired Rₐ is COOR_{y} or CONR_{w}Rₓ, these can be derived from the corresponding hydroxyl group. Oxidation of the hydroxyl group to the acid followed by ester or amide formation under conditions known in the art can provide examples wherein Rₐ is COOR_{y} or CONR_{w}Rₓ. Corresponding compounds of Formula II can be prepared by the same method outlined in Scheme 8 using the appropriate 6-bromo-4-chloro-thieno[2,3-d]pyrimidine or pyridine.

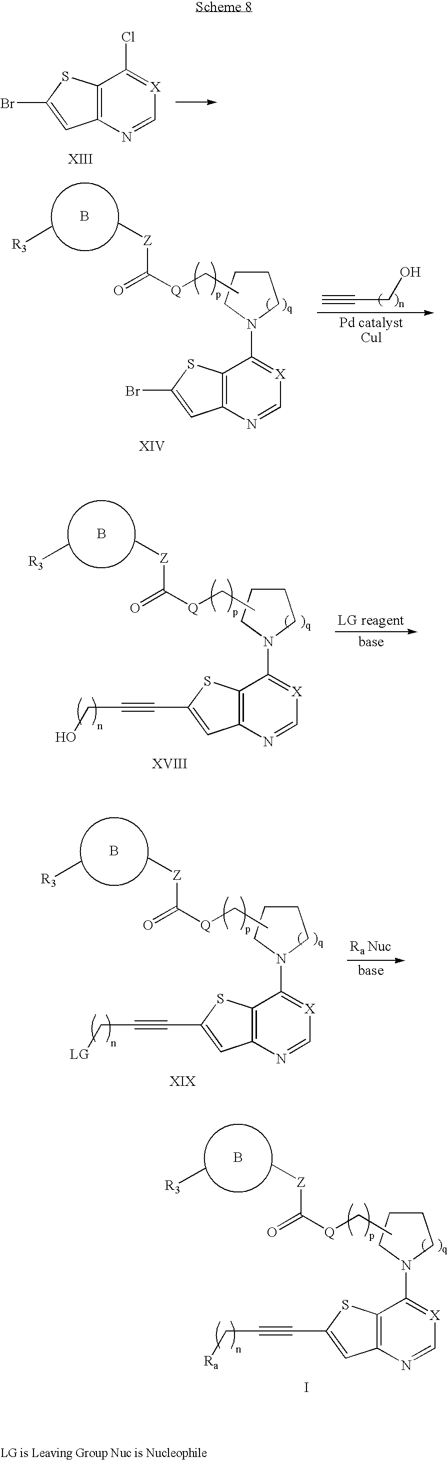

Representative Compounds

Representative compounds of the present invention synthesized by the aforementioned methods are presented below. Examples of the synthesis of specific compounds are presented thereafter. Preferred compounds are numbers 5, 9, 11, 15, 18, 19, 25 and 26; particularly preferred are numbers 5, 9, 11, 25, and 26.

| Number | Compound |
|---|---|
| 1 | 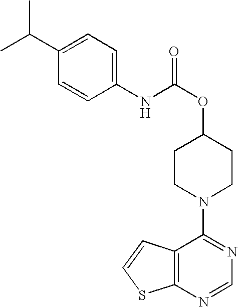 |
| 2 | 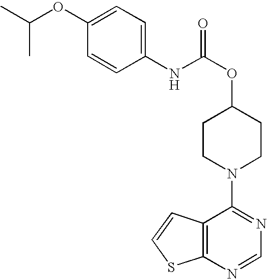 |
| 3 | 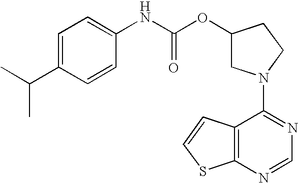 |
| 4 | 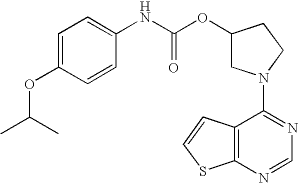 |
| 5 | 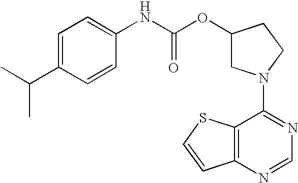 |
| 6 | 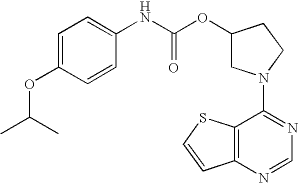 |
| 7 | 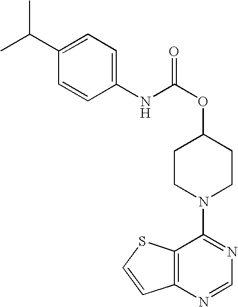 |
| 8 | 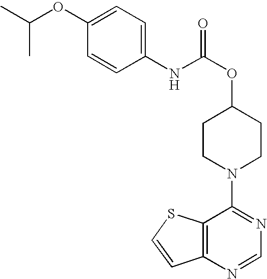 |
| 9 | 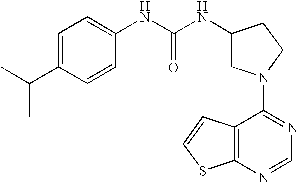 |
| 10 | 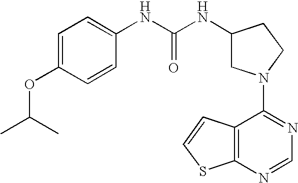 |
| 11 | 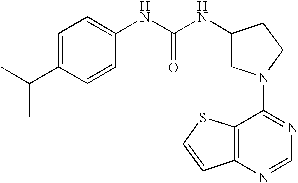 |
| 12 | 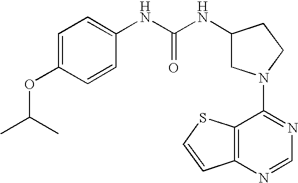 |
| 13 | 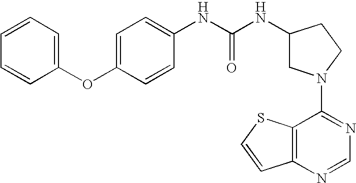 |
| 14 | 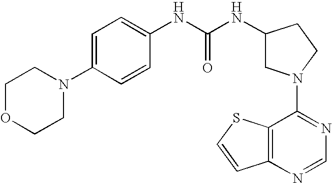 |
| 15 | 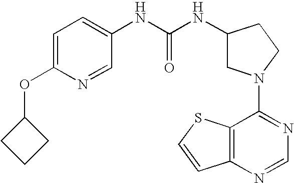 |
| 16 | 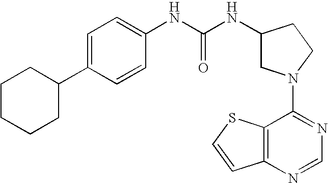 |
| 17 | 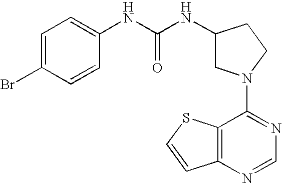 |
| 18 | 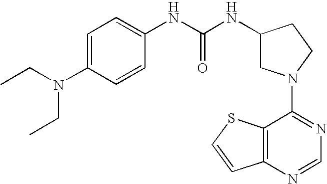 |
| 19 | 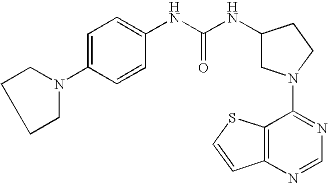 |
| 20 | 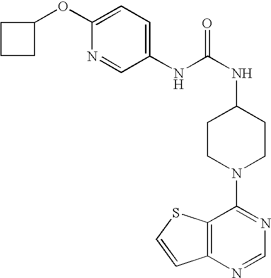 |
| 21 | 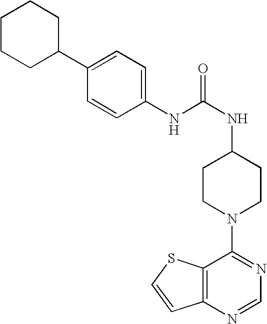 |
| 22 | 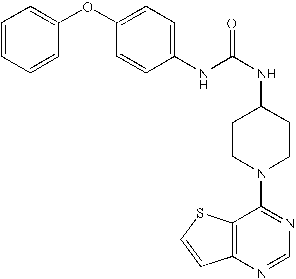 |
| 23 | 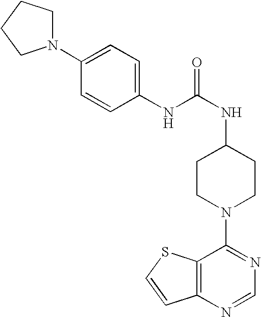 |
| 24 | 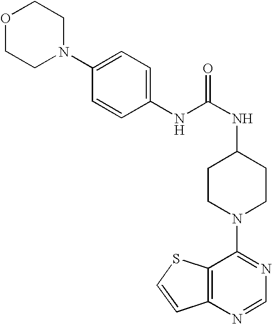 |
| 25 | 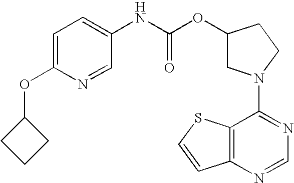 |
| 26 | 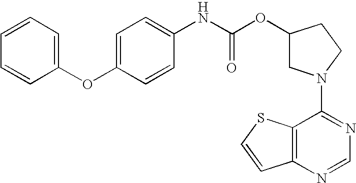 |
| 27 | 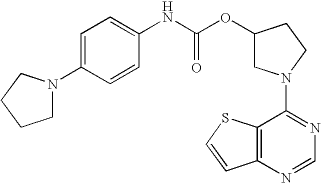 |
| 28 | 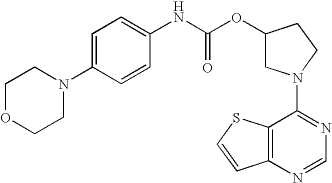 |
| 29 | 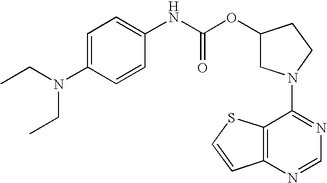 |

### EXAMPLE 1

### (4-Isopropyl-phenyl)-carbamic acid 1-thieno[2,3-d]pyrimidin-4-yl-piperidin-4-yl ester

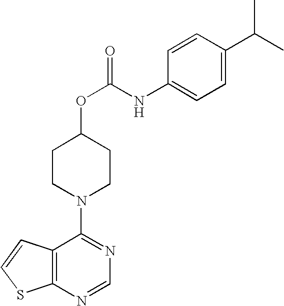

a. 1-Thieno[2,3-d]pyrimidin-4-yl-piperidin-4-ol

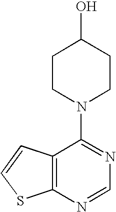

A solution of 4-chloro-thieno[2,3-d]pyrimidine (85.3 mg, 0.502 mmol) in isopropanol (2 mL) was treated with 4-hydroxypiperidine (50.6 mg, 0.501 mmol). After stirring at 100° C, overnight, the reaction was cooled to RT, partitioned between DCM (20 mL) and H₂0 (20 mL). The organic phase was dried over Na₂SO₄ and concentrated in vacuo to afford the title compound as a solid (67.8 mg, 58%), which was used in the next step without further purification or characterization.

b. (4-Isopropyl-phenyl)-carbamic acid 1-thieno [2,3-d]pyrimidin-4-yl-piperidin-4-yl ester

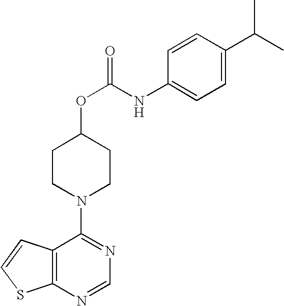

To a solution of 1,1′-carbonyldiimidazole (23.5 mg, 0.145 mmol) in DCM (1 mL) was added 4-isopropylaniline (19.6 mg, 0.145 mmol). After stirring at 0° C. for 2 h, 1-thieno[2,3-d]pyrimidin-4-yl-piperidin-4-ol (34.1 mg, 0.145 mmol), as prepared in the previous step, was added and stirred at RT. After 2 h, DMAP (17.7 mg, 0.145 mmol) was added and stirred at 85° C. overnight. The reaction was then cooled to RT, partitioned between DCM (10 mL) and H₂0 (10 mL). The organic phase was dried over Na₂SO₄ and concentrated in vacuo. Purification by prep tlc (1:1 Hexane/EtOAc) afforded the title compound as a light brown solid (9.8 mg, 17%). ¹H NMR (300 MHz, CDCl₃) δ 8.7 (br s, 1H), 7.46 (br m, 1H), 7.30 (m, 3H), 7.17 (m, 2H), 6.65 (br s, 1H), 5.10 (m, 1H), 4.18 (m, 2H), 3.75 (m, 2H), 2.88 (heptet, 1H), 2.12 (m, 2H), 1.87 (m, 2H), 1.23 (d, 6H). LC/MS (ESI): calcd mass 396.2, found 397.2 [M+1]⁺.

### EXAMPLE 2

### (4-Isopropoxy-phenyl)-carbamic acid 1-thieno[2,3-d]pyrimidin-4-yl-piperidin-4-yl ester

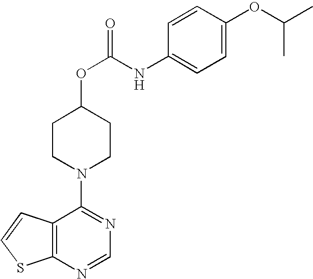

To a solution of 1,1′-carbonyldiimidazole (23.3 mg, 0.144 mmol) in DCM (1 mL) was added 4-isopropoxyaniline (21.7 mg, 0. 144 mmol). After stirring at 0° C. for 2 h, 1-thieno[2,3-d]pyrimidin-4-yl-piperidin-4-ol (33.7 mg, 0.143 mmol), as prepared in Example 1a, was added and stirred at RT. After 2 h, DMAP (17.6 mg, 0.144 mmol) was added and stirred at 85° C. overnight. The reaction was then cooled to RT, partitioned between DCM (10 mL) and H₂0 (10 mL). The organic phase was dried over Na₂SO₄ and concentrated in vacuo. Purification by prep tlc (1:1 Hexane/EtOAc) afforded the title compound as a light green solid (8.4 mg, 14%). ¹H NMR (300 MHz, CDCl₃) δ 8.7 (br s, 1H), 7.44 (br m, 1H), 7.29 (m, 3H), 6.85 (m, 2H), 6.56 (br s, 1H), 5.09 (m, 1H), 4.48 (heptet, 1H), 4.17 (m, 2H), 3.75 (m, 2H), 2.11 (m, 2H), 1.87 (m, 2H), 1.31 (d, 6H). LC/MS (ESI): calcd mass 412.2, found 413.2 [M+1]⁺.

### EXAMPLE 3

### (4-Isopropyl-phenyl)-carbamic acid 1-thieno[2,3-d]pyrimidin-4-yl-pyrrolidin-3-yl ester

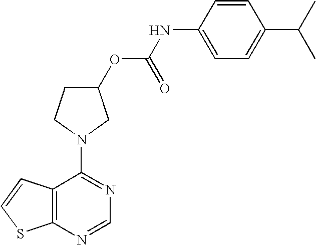

a. (4-Isopropyl-phenyl)-carbamic acid 4-nitro-phenyl ester

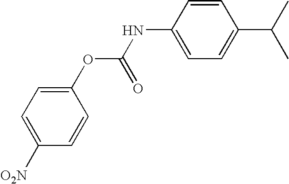

To a solution of 4-isopropylaniline (3.02 g, 22.3 mmol) in DCM (40 mL) and pyridine (10 mL) was added 4-nitrophenyl chloroformate (4.09 g, 20.3 mmol) portionwise with stirring over 30 sec with brief ice-bath cooling. After stirring at rt for 1 h, the homogeneous solution was diluted with DCM (100 mL) and washed with 0.6 M HCl (1×250 mL), 0.025 M HCl (1×400 mL), water (1×100 mL), and 1 M NaHCO₃ (1×100 mL). The organic layer was dried (Na₂SO₄) and concentrated to give the title compound as a light peach-colored solid (5.80 g, 95%). ¹H NMR (300 MHz, CDCl₃) δ 8.28 (m, 2H), 7.42-7.32 (m, 4H), 7.23 (m, 2H), 6.93 (br s, 1H), 2.90 (h, J=6.9 Hz, 1H), 1.24 (d, J=6.9 Hz, 6H). LC/MS (ESI): calcd mass 300.1, found 601.3 (2MH)⁺.

b. (4-Isopropyl-phenyl)-carbamic acid 1-thieno[2,3-d]pyrimidin-4-yl-pyrrolidin-3-yl ester

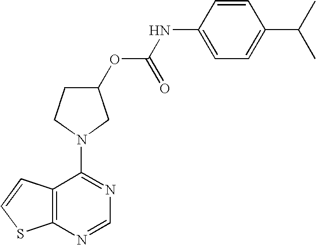

A mixture of pyrrolidin-3-ol (15.3 mg, 176 μmol), 4-chloro-thieno[2,3-d]pyrimidine (30.3 mg, 178 μmol) (Maybridge), DIEA (32 μL, 194 μmol), and DMSO-d₆ (117 μL) was stirred at 80° C. for 1 h. The reaction was then allowed to cool to rt, (4-isopropyl-phenyl)-carbamic acid 4-nitro-phenyl ester (68.1 mg, 227 μmol), as prepared in the previous step, was added, followed by NaH (dry) (5.4 mg, 225 μmol). The mixture was stirred (loosely capped) at rt for 5 min until the majority of gas evolution had subsided, and was then stirred at 80° C. for 20 min. The reaction was allowed to cool to rt, shaken with 2.0 M K₂CO₃ (1×2 mL), and extracted with DCM (2×2 mL), with phases separated by centrifugal force. The organic layers were combined, dried (Na₂SO₄), and concentrated. Flash chromatography of the residue (3:1 EtOAc/hex) provided the title compound as an off-white powder (49.1 mg, 72%). ¹H NMR (300 MHz, CDCl₃) δ 8.47 (s, 1H), 7.46 (d, 1H), 7.28 (m, 2H), 7.22 (d, 1H), 7.16 (m, 2H), 6.67 (br s, 1 H), 5.53 (m, 1H), 4.12-3.90 (m, 4H), 2.86 (heptet, 1H), 2.43-2.22 (m, 2H), 1.22 (d, 6H). LC/MS (ESI): calcd mass 382.2, found 383.2 (MH)⁺.

### EXAMPLE 4

### (4-Isopropoxy-phenyl)-carbamic acid 1-thieno[2,3-d]pyrimidin-4-yl-pyrrolidin-3-yl ester

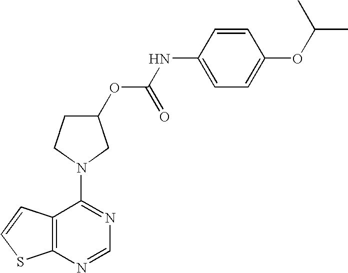

a. (4-Isopropoxy-phenyl)-carbamic acid 4-nitro-phenyl ester

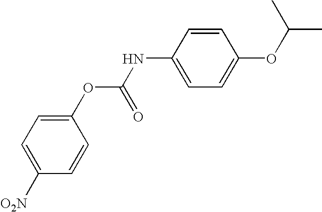

Prepared essentially as described for Example 3a using 4-isopropoxyaniline, except the water and 1M NaHCO₃ washes were omitted. The title compound was obtained as a light violet-white solid (16.64 g, 98%). ¹H NMR (300 MHz, CDCl₃) δ 8.26 (m, 2H), 7.40-7.28 (m, 4H), 6.98 (br s, 1H), 6.87 (m, 2H), 4.50 (heptet, J=6.0 Hz, 1H), 1.33 (d, J=6.0 Hz, 6H). LC/MS (ESI): calcd mass 316.1, found 633.2 (2MH)⁺.

b. (4-Isopropoxy-phenyl)-carbamic acid 1-thieno [2,3-d]pyrimidin-4-yl-pyrrolidin-3-yl ester

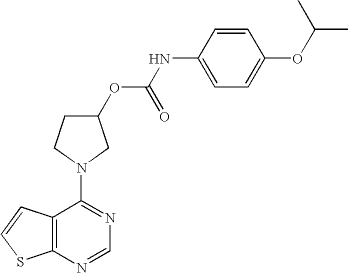

Prepared essentially as described for Example 3b using 4-chloro-thieno[2,3-d]pyrimidine (Maybridge) and (4-isopropoxy-phenyl)-carbamic acid 4-nitro-phenyl ester (prepared in the previous step), except the SNAr reaction was performed at 80° C. for 1 h, and 1.6 eq NaH was used. Flash chromatography (3:1 EtOAc/hex) provided the title compound as an off-white powder (44.3 mg, 73%). ¹H NMR (300 MHz, CDCl₃) δ 8.47 (s, 1H), 7.46 (d, J=6.1 Hz, 1H), 7.28-7.21 (m, 3H), 6.83 (m, 2H), 6.55 (br s, 1H), 5.52 (m, 1H), 4.47 (heptet, J=6.1 Hz, 1H), 4.14-3.90 (m, 4H), 2.43-2.20 (m, 2H), 1.31 (d, J=6.1 Hz, 6H). LC/MS (ESI): calcd mass 398.1, found 399.2 (MH)⁺.

### EXAMPLE 5

### (4-Isopropyl-phenyl)-carbamic acid 1-thieno [3,2-d]pyrimidin-4-yl-pyrrolidin-3-yl ester

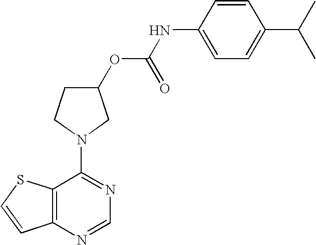

Prepared essentially as described for Example 3b using 4-chloro-thieno[3,2-d]pyrimidine (Maybridge) and (4-isopropyl-phenyl)-carbamic acid 4-nitro-phenyl ester (prepared in Example 3a), except the SNAr reaction was performed at 80° C. for 1 h. Flash chromatography (3:4 hex/acetone) provided the title compound (38.5 mg, 59%). ¹H NMR (300 MHz, CDCl₃) δ 8.53 (s, 1H), 7.75 (d, 1H), 7.42 (d, 1H), 7.28 (m, 2H), 7.16 (m, 2H), 6.74 (br s, 1H), 5.53 (m, 1H), 4.21-3.92 (m, 4H), 2.87 (heptet, 1H), 2.43-2.22 (m, 2H), 1.22 (d, 6H). LC/MS (ESI): calcd mass 382.2, found 383.2 (MH)⁺.

### EXAMPLE 6

### (4-Isopropoxy-phenyl)-carbamic acid 1-thieno[3,2-d]pyrimidin-4-yl-pyrrolidin-3-yl ester

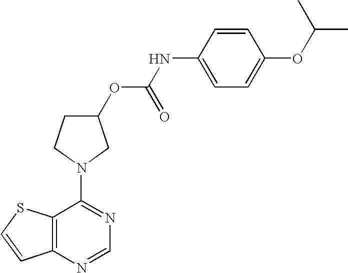

Prepared essentially as described for Example 3b using 4-chloro-thieno[3,2-d]pyrimidine (Maybridge) and (4-isopropoxy-phenyl)-carbamic acid 4-nitro-phenyl ester (prepared in Example 4a), except the SNAr reaction was performed at 80° C. for 1 h. Flash chromatography (3:4 hex/acetone) provided the title compound (43.1 mg, 69%). ¹H NMR (300 MHz, CDCl₃) δ 8.54 (s, 1H), 7.76 (d, 1H), 7.43 (d, 1H), 7.25 (m, 2H), 6.83 (m, 2H), 6.60 (br s, 1H), 5.52 (m, 1H), 4.48 (heptet, 1H), 4.22-3.92 (m, 4H), 2.43-2.22 (m, 2H), 1.31 (d, 6H). LC/MS (ESI): calcd mass 398.1, found 399.2 (MH)⁺.

### EXAMPLE 7

### (4-Isopropyl-phenyl)-carbamic acid 1-thieno[3,2-d]pyrimidin-4-yl-piperidin-4-yl ester

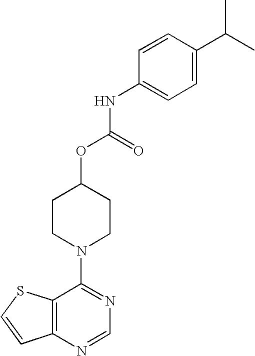

Prepared essentially as described for Example 3b using 4-chloro-thieno[3,2-d]pyrimidine (Maybridge), 4-hydroxypiperidine (Acros, less than 1% water, K.F.), and (4-isopropyl-phenyl)-carbamic acid 4-nitro-phenyl ester (prepared in Example 3a), except 1.4 eq NaH was used. Flash chromatography (1:4 hex/EtOAc) provided the title compound (23.7 mg, 31%). ¹H NMR (300 MHz, CDCl₃) δ 8.60 (s, 1H), 7.75 (d, 1H), 7.46 (d, 1H), 7.35-7.25 (m, 2H), 7.18 (m, 2H), 6.60 (br s, 1H), 5.10 (m, 1H), 4.36-4.25 (m, 2H), 3.92-3.80 (m, 2H), 2.88 (heptet, 1H), 2.20-2.07 (m, 2H), 1.93-1.80 (m, 2H), 1.23 (d, 6H). LC/MS (ESI): calcd mass 396.2, found 397.2 (MH)⁺.

### EXAMPLE 8

### (4-Isopropoxy-phenyl)-carbamic acid 1-thieno[3,2-d]pyrimidin-4-yl-piperidin-4-yl ester

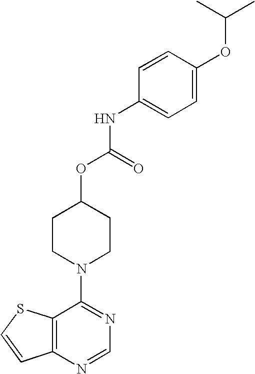

Prepared essentially as described for Example 3b using 4-chloro-thieno[3,2-d]pyrimidine (Maybridge), 4-hydroxypiperidine (Acros, less than 1% water, K.F.), and (4-isopropoxy-phenyl)-carbamic acid 4-nitro-phenyl ester (prepared in Example 4a), except 1.7 eq NaH was used. Flash chromatography (1:4 hex/EtOAc) provided the title compound (42.1 mg, 62%). ¹H NMR (300 MHz, CDCl₃) δ 8.60 (s, 1H), 7.74 (d, 1H), 7.44 (d, 1H), 7.29 (m, 2H), 6.85 (m, 2H), 6.59 (br s, 1H), 5.09 (m, 1H), 4.49 (heptet, 1H), 4.35-4.21 (br m, 2H), 3.91-3.79 (m, 2H), 2.17-2.05 (m, 2H), 1.92-1.78 (m, 2H), 1.32 (d, 6H). LC/MS (ESI): calcd mass 412.2, found 413.2 (MH)⁺.

### EXAMPLE 9

### 1-(4-Isopropyl-phenyl)-3-(1-thieno [2,3-d]pyrimidin-4-yl-pyrrolidin-3-yl)-urea

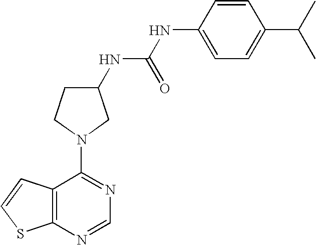

To a mixture of 4-chloro-thieno[2,3-d]pyrimidine (Maybridge) (25.4 mg, 149 μmol), 3-(tert-butoxycarbonylamino)pyrrolidine (TCI America) (27.1 mg, 146 μmol), and DIEA (27.5 μL, 166 μmol) was added DMSO (100 μL), and the reaction was stirred at 100° C. for 20 min. The reaction solution was allowed to cool to rt, TFA (230 μL, 2.98 mmol) was added in one portion, and the reaction stirred at 100° C. for 5 min. After cooling to rt, the reaction was partitioned with DCM (2 mL) and 2.5 M NaOH (2 mL), and the organic layer was collected and concentrated without drying to give the intermediate pyrrolidinylamine which was used immediately for the next step without further purification or characterization. To this intermediate was added (4-isopropyl-phenyl)-carbamic acid 4-nitro-phenyl ester (58.8 mg, 196 μmol), prepared as described in Example 3a, and CH₃CN (100 μL), and the reaction was heated at 100° C. for 15 min. After cooling to rt, the reaction was partitioned with DCM (2 mL) and 2 M K₂CO₃ (2 mL), the aqueous layer was extracted with DCM (1×2 mL), and the organic layers were combined, dried (Na₂SO₄), and concentrated. Purification with silica flash chromatography (1:1 hex/acetone) afforded the title compound (26.3 mg, 47%). ¹H NMR (300 MHz, CDCl₃) δ 8.28 (s, 1H), 7.26-7.21 (m, 3H), 7.13 (m, 2H), 7.09 (d, 1H), 7.00 (br s, 1H), 6.25 (br d, 1H), 4.60 (m, 1H), 3.96-3.79 (m, 4H), 2.84 (heptet, 1H), 2.30-2.14 (m, 2H), 1.20 (d, 6H). LC/MS (ESI): calcd mass 381.2, found 382.2 (MH)⁺.

### EXAMPLE 10

### 1-(4-Isopropoxy-phenyl)-3-(1-thieno[2,3-d]pyrimidin-4-yl-pyrrolidin-3-yl)-urea

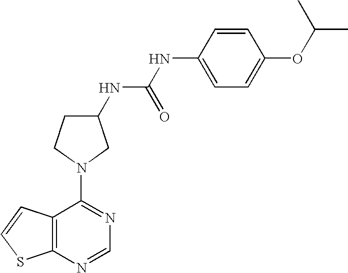

Prepared essentially as described for Example 9, using (4-isopropoxy-phenyl)-carbamic acid 4-nitro-phenyl ester as prepared in Example 4a. Flash chromatography (1:1 hex/acetone) afforded the title compound (25.8 mg, 45%). ¹H NMR (300 MHz, CDCl₃) δ 8.31 (s, 1H), 7.29 (d, 1H), 7.19 (m, 2H), 7.11 (d, 1H), 6.81 (m, 2H), 6.75 (br s, 1H), 5.90 (br d, 1H), 4.59 (m, 1H), 4.46 (heptet, 1H), 4.02-3.90 (m, 1H), 3.89-3.76 (m, 3H), 2.32-2.15 (m, 2H), 1.30 (d, 6H). LC/MS (ESI): calcd mass 397.2, found 398.2 (MH)⁺.

### EXAMPLE 11

1-(4-Isopropyl-phenyl)-3-(1-thieno [3,2-d]pyrimidin-4-yl-pyrrolidin-3-yl)-urea

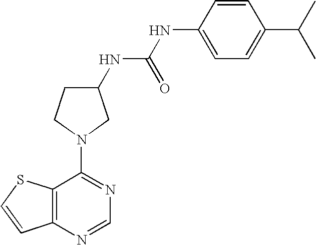

Prepared essentially as described for Example 9, using 4-chloro-thieno[3,2-d]pyrimidine (Maybridge). Flash chromatography (1:2 hex/acetone) afforded the title compound (17.2 mg, 30%). ¹H NMR (300 MHz, CDCl₃) δ 8.32 (s, 1H), 7.71 (d, 1H), 7.36 (br s, 1H), 7.34 (d, 1H), 7.27 (m, 2H), 7.12 (m, 2H), 6.76 (br d, 1H), 4.62 (m, 1H), 3.87-3.66 (m, 4H), 2.84 (heptet, 1H), 2.32-2.15 (m, 2H), 1.20 (d, 6H). LC/MS (ESI): calcd mass 381.2, found 382.2 (MH)⁺.

### EXAMPLE 12

### 1-(4-Isopropoxy-phenyl)-3-(1-thieno[3,2-d]pyrimidin-4-yl-pyrrolidin-3-yl)-urea

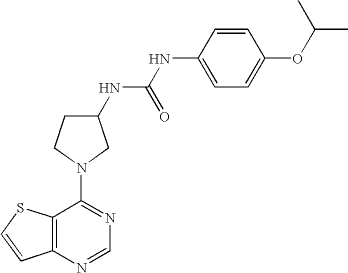

Prepared essentially as described for Example 9, using 4-chloro-thieno[3,2-d]pyrimidine (Maybridge) and (4-isopropoxy-phenyl)-carbamic acid 4-nitro-phenyl ester as prepared in Example 4a. Flash chromatography (1:2→1:3 hex/acetone) afforded the title compound (23.3 mg, 39%). ¹H NMR (300 MHz, CDCl₃) δ 8.34 (s, 1H), 7.71 (d, 1H), 7.34 (d, 1H), 7.22 (m, 2H), 7.14 (br s, 1H), 6.81 (m, 2H), 6.48 (br d, 1H), 4.60 (m, 1H), 4.45 (heptet, 1H), 3.94-3.74 (m, 4H), 2.27-2.16 (m, 2H), 1.30 (d, 6H). LC/MS (ESI): calcd mass 397.2, found 398.2 (MH)⁺.

### EXAMPLE 13

1-(4-Phenoxy-phenyl)-3-(1-thieno [3,2-d]pyrimidin-4-yl-pyrrolidin-3-yl)-urea

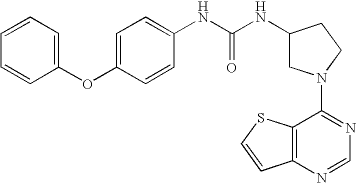

a. (1-Thieno[3,2-d]pyrimidin-4-yl-pyrrolidin-3-yl)-carbamic acid tert-butyl ester

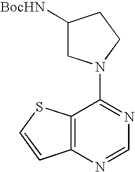

A solution of 4-chlorothieno[3,2-d]pyimidine (400 mg, 2.35 mmol), Pyrrolidine-3-yl-carbamic acid tert-butyl ester (436 mg, 2.35 mmol), diisopropylethylamine (285 mg, 2.82 mmol) in isopropanol (10 mL) was heated to 100° C. for 1 hr. The resulting mixture was cooled to RT, poured into ethyl acetate (50 mL), and washed with water (25 mL). The organic layer was dried over anhydrous sodium sulfate, concentrated, and purified by silica gel chromatography (5% MeOH/EtOAc) to provide the title compound (645 mg, 86% yield). ¹H NMR (400 MHz, CD₃OD) δ 8.34 (s, 1H), 8.02 (d, 1H), 7.32 (d, 1H), 4.23 (m, 1H), 4.18-3.92 (m, 3H), 3.78 (m, 1H), 2.26 (m, 1H), 2.04 (m, 1H), 1.42 (s, 9H). LC/MS (ESI): calcd mass 320.1, found 321.2 (MH)⁺.

b. 1-Thieno[3,2-d]pyrimidin-4-yl-pyrrolidin-3-ylamine hydrochloride

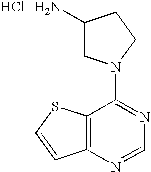

A solution of (1-Thieno[3,2-d]pyrimidin-4-yl-pyrrolidin-3-yl)-carbamic acid tert-butyl ester (645 mg, 2.02 mmol), 2 M HCl/Et₂O (4 mL), and CH₂Cl₂ (20 mL) was stirred at RT for 16 h. The resulting solid was filtered and washed with EtOAc to provide the title compound as an off-white solid (491 mg, 95%). LC/MS (ESI): calcd mass 220.1, found 221.1 (MH)⁺.

c. 1-(4-Phenoxy-phenyl)-3-(1-thieno[3,2-d]pyrimidin-4-yl-pyrrolidin-3-yl)-urea

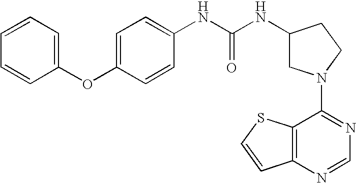

To a solution of 1-Thieno[3,2-d]pyrimidin-4-yl-pyrrolidin-3-ylamine hydrochloride (21 mg, 0.082 mmol) and diisopropylethylamine (17.3 mg, 0.172 mmol) in CH₂Cl₂ (0.5 mL) was added 4-phenoxyphenyl isocyanate (21 mg, 0.99 mmol). The resulting solution was stirred at RT for 16 h, then poured into 1 M HCl (5 mL) and extracted with CH₂Cl₂ (10 mL). The organic layer was dried over anhydrous sodium sulfate, concentrated, and purified by silica gel chromatography (2% MeOH/CH₂CH₂) to provide the title compound (17 mg) as a white solid. ¹H NMR (400 MHz, CD₃OD) δ 8.36 (s, 1H), 8.05 (d, 1H), 7.35-7.28 (m, 5H), 7.05 (m, 1H), 6.92 (m, 4H), 4.49 (m, 1H), 4.22-3.98 (m, 3H), 3.87 (m, 1H), 2.36 (m, 1H), 2.11 (m, 1H). LC/MS (ESI): calcd mass 431.1, found 432.1 (MH)⁺.

### EXAMPLE 14

### 1-(4-Morpholin-4-yl-phenyl)-3-(1-thieno [3,2-d]pyrimidin-4-yl-pyrrolidin-3-yl)-urea

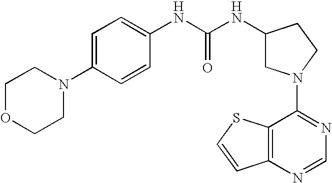

a. (4-Morpholin-4-yl-phenyl)-carbamic acid 4-nitro-phenyl ester; hydrochloride

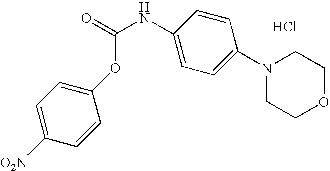

A solution of 4-nitrophenyl chloroformate (798 mg, 3.96 mmol) in THF (2.0 mL) was added rapidly by syringe over 10 s at rt under air to a stirred solution of 4-morpholin-4-yl-phenylamine (675 mg, 3.79 mmol) in THF (8.8 mL), with a heavy grey precipitate forming “instantly”. The reaction was immediately capped and stirred “rt” for 30 min (vial spontaneously warmed), and was then filtered. The grey filter cake was washed with dry THF (2×10 mL), and dried under high vacuum at 80° C. to afford the title compound as a grey powder (1.361 g, 95%). A portion was partitioned with CDCl₃ and aqueous 0.5 M trisodium citrate to generate the CDCl₃-soluble free base: ¹H-NMR (300 MHz, CDCl₃) δ 8.28 (m, 2H), 7.42-7.31 (m, 4H), 6.95-6.88 (m, 3H), 3.87 (m, 4H), 3.14 (m, 4H).

b. 1-(4-Morpholin-4-yl-phenyl)-3-(1-thieno[3,2-d]pyrimidin-4-yl-pyrrolidin-3-yl)-urea

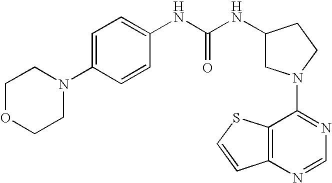

A solution of 1-Thieno[3,2-d]pyrimidin-4-yl-pyrrolidin-3-ylamine hydrochloride (15 mg, 0.059 mmol), prepared as described in Example 13b, diisopropylethylamine (12.4 mg, 0.123 mmol), (4-Morpholin-4-yl-phenyl)-carbamic acid 4-nitro-phenyl ester hydrochloride (22.2 mg, 0.059 mmol) and acetonitrile (0.5 mL) was heated at 90° C. for 2 h. The resulting solution was poured into CH₂Cl₂ (10 mL) and washed sequentially with 1 M NaOH (5 mL) and H₂0 (5 mL). The organic layer was dried over anhydrous sodium sulfate, concentrated, and purified by silica gel chromatography (2% MeOH/CH₂Cl₂) to provide the title compound (15 mg) as a white solid. ¹H NMR (400 MHz, CD₃OD) δ 8.35 (s, 1H), 8.04 (d, 1H), 7.34 (d, 1H), 7.23 (m, 2H), 6.90 (m, 2H), 4.48 (m, 1H), 4.22-3.97 (m, 3H), 3.87-3.79 (m, 5H), 3.03 (m, 4H), 2.35 (m, 1H), 2.10 (m, 1H). LC/MS (ESI): calcd mass 424.2, found 425.1 (MH)⁺.

### EXAMPLE 15

### 1-(6-Cyclobutoxy-pyridin-3-yl)-3-(1-thieno [3,2-d]pyrimidin-4-yl-pyrrolidin-3-yl)-urea

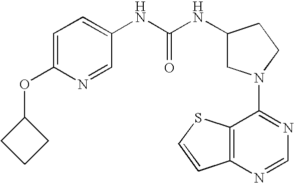

a. 2-Cyclobutoxy-5-nitro-pyridine

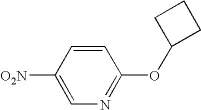

A mixture of 2-chloro-5-nitropyridine (7.12 g, 45.0 mmol) and cyclobutanol (3.40 g, 47.2 mmol) in THF (30 mL) was vigorously stirred at 0° C. while NaH (1.18 g, 46.7 mmol) was added in three portions over 10-20 s under air (Caution: Extensive gas evolution). Reaction residue was rinsed down with additional THF (5 mL), followed by stirring under positive argon pressure in the ice bath for 1-2 more minutes. The ice bath was then removed and the brown homogeneous solution was stirred at “rt” for 1 h. The reaction was concentrated under reduced pressure at 80° C., taken up in 0.75 M EDTA (tetrasodium salt) (150 mL), and extracted with DCM (1×100 mL, 1×50 mL). The combined organic layers were dried (Na₂SO₄), concentrated, taken up in MeOH (2×100 mL) and concentrated under reduced pressure at 60° C. to provide the title compound as a thick dark amber oil that crystallized upon standing (7.01 g, 80%). ¹H NMR (300 MHz, CDCl₃) δ 9.04 (dd, J=2.84 and 0.40 Hz, 1H), 8.33 (dd, J=9.11 and 2.85 Hz, 1H), 6.77 (dd, J=9.11 and 0.50 Hz, 1H), 5.28 (m, 1H), 2.48 (m, 2H), 2.17 (m, 2H), 1.87 (m, 1H), 1.72 (m, 1H).

b. 6-Cyclobutoxy-pyridin-3-ylamine

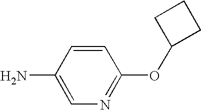

A flask containing 10% w/w Pd/C (485 mg) was gently flushed with argon while slowly adding MeOH (50 mL) along the sides of the flask, followed by the addition in −5 mL portions of a solution of 2-cyclobutoxy-5-nitro-pyridine (4.85 g, 25 mmol), as prepared in the previous step, in MeOH (30 mL). (Caution: Large scale addition of volatile organics to Pd/C in the presence of air can cause fire.) The flask was then evacuated one time and stirred under H2 balloon pressure for 2 h at rt. The reaction was then filtered, and the clear amber filtrate was concentrated, taken up in toluene (2×50 mL) to remove residual MeOH, and concentrated under reduced pressure to provide the crude title compound as a translucent dark brown oil with a faint toluene smell (4.41 g, “108%” crude yield). ¹H NMR (300 MHz, CDCl₃) δ 7.65 (d, J=3.0 Hz, 1H), 7.04 (dd, J=8.71 and 2.96 Hz, 1H), 6.55 (d, J=8.74 Hz, 1H), 5.04 (m, 1H), 2.42 (m, 2H), 2.10 (m, 2H), 1.80 (m, 1H), 1.66 (m, 1H). LC-MS (ESI): calcd mass 164.1, found 165.2 (MH⁺).

c. (6-Cyclobutoxy-pyridin-3-yl)-carbamic acid 4-nitro-phenyl ester

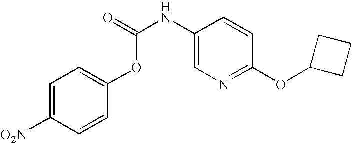

A mixture of 6-cyclobutoxy-pyridin-3-ylamine (4.41 g, assume 25 mmol), as prepared in the previous step, and CaCO₃ (3.25 g, 32.5 mmol) (10 micron powder) was treated with a homogeneous solution of 4-nitrophenyl chloroformate (5.54 g, 27.5 mmol) in toluene (28 mL) in one portion at rt, and was stirred at “rt” (reaction warmed spontaneously) for 2 h. The reaction mixture was then directly loaded onto a flash silica column (95:5 DCM/MeOH→9:1 DCM/MeOH) to afford 5.65 g of material, which was further purified by trituration with hot toluene (1×200 mL) to provide the title compound (4.45 g, 54%). ¹H NMR (400 MHz, CDCl₃) δ 8.28 (m, 2H), 8.12 (d, 1H), 7.81 (m, 1H), 7.39 (m, 2H), 6.85 (br s, 1H), 6.72 (d, 1H), 5.14 (m, 1H), 2.45 (m, 2H), 2.13 (m, 2H), 1.84 (m, 1H), 1.68 (m, 1H). LC-MS (ESI): calcd mass 329.1, found 330.1 (MH⁺).

d. 1-(6-Cyclobutoxy-pyridin-3-yl)-3-(1-thieno [3,2-d]pyrimidin-4-yl-pyrrolidin-3-yl)-urea

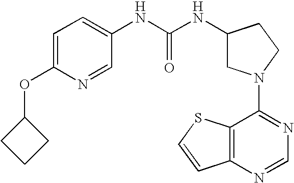

Prepared essentially as described in Example 14 using (6-Cyclobutoxy-pyridin-3-yl)-carbamic acid 4-nitro-phenyl ester in place of (4-morpholin-4-yl-phenyl)-carbamic acid 4-nitrophenyl ester hydrochloride. ¹H NMR (400 MHz, CD₃OD) δ 8.35 (s, 1H), 8.05 (m, 2H), 7.71 (dd, 1H), 7.33 (d, 1H), 6.68 (d, 1H), 5.02 (m, 1H), 4.47 (m, 1H), 4.22-3.97 (m, 3H), 3.85 (m, 1H), 2.47-2.31 (m, 3H), 2.08 (m, 3H), 1.82 (m, 1H), 1.69 (m, 1H). LC/MS (ESI): calcd mass 410.2, found 411.1 (MH)⁺.

### EXAMPLE 16

1-(4-Cyclohexyl-phenyl)-3-(1-thieno [3,2-d]pyrimidin-4-yl-pyrrolidin-3-yl)-urea

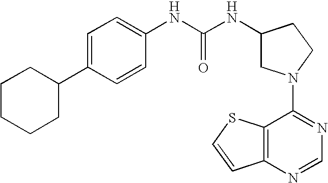

a. (4-Cyclohexyl-phenyl)-carbamic acid 4-nitro-phenyl ester

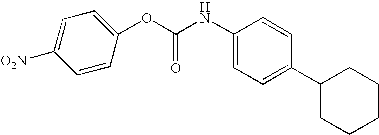

Prepared essentially as described in Example 3a except that 4-cyclohexylaniline was used in place of 4-isopropylaniline.¹H NMR (DMSO-d₆) δ 10.37 (br, 1H), 8.30 (d, J=9.30 Hz, 2H), 7.52 (d, J=9.00 Hz, 2H), 7.41 (d, J=8.10 Hz, 2H), 7.18 (d, J=8.70 Hz, 2H), 1.18-1.82 (11H).

b. 1-(4-Cyclohexyl-phenyl)-3-(1-thieno [3,2-d]pyrimidin-4-yl-pyrrolidin-3-yl)-urea

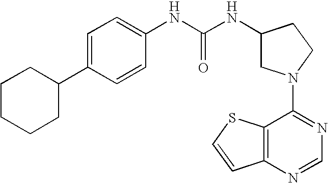

Prepared essentially as described in Example 14 using (4-Cyclohexyl-phenyl)-carbamic acid 4-nitro-phenyl ester in place of (4-morpholin-4-yl-phenyl)-carbamic acid 4-nitrophenyl ester hydrochloride. ¹H NMR (400 MHz, CD₃OD) δ 8.36 (s, 1H), 8.05 (d, 1H), 7.34 (d, 1H), 7.23 (m, 2H), 7.09 (m, 2H), 4.48 (m, 1H), 4.22-3.98 (m, 3H), 3.86 (m, 1H), 2.46-2.32 (m, 2H), 2.10 (m, 1H), 1.84-1.71 (m,5H), 1.47-1.22 (m, 5H). LC/MS (ESI): calcd mass 421.2, found 422.1 (MH)⁺.

### EXAMPLE 17

1-(4-Bromo-phenyl)-3-(1-thieno [3,2-d]pyrimidin-4-yl-pyrrolidin-3-yl)-urea

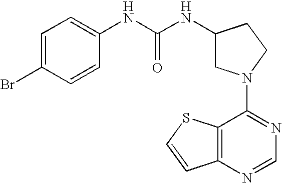

Prepared essentially as described in Example 13 using 4-bromophenyl isocyanate in place of 4-phenoxyphenyl isocyanate. ¹H NMR (400 MHz, CD₃OD) δ 8.36 (s, 1H), 8.05 (d, 1H), 7.38-7.29 (m, 5H), 4.48 (m, 1H), 4.22-3.98 (m, 3H), 3.87 (m, 1H), 2.37 (m, 1H), 2.12 (m, 1H). LC/MS (ESI): calcd mass 417.0, found 419.9 (MH)⁺.

### EXAMPLE 18

### 1-(4-Diethylamino-phenyl)-3-(1-thieno [3,2-d]pyrimidin-4-yl-pyrrolidin-3-yl)-urea

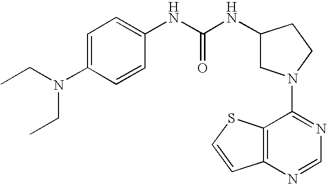

a. (4-Diethylamino-phenyl)-carbamic acid 4-nitro-phenyl ester hydrochloride

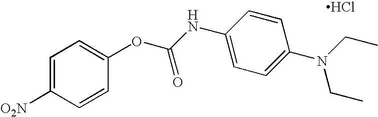

A solution of N,N-diethyl-benzene-1,4-diamine (2.21 g, 13.5 mmol) in DCM (30 mL) was added rapidly dropwise under air over two minutes to a stirred solution of 4-nitrophenyl chloroformate (2.86 g, 14.2 mmol) in DCM (7.4 mL) in an open beaker with rt water bath cooling. The resulting mixture was stirred at rt for 30 min, then filtered. The filter cake was powdered with mortar and pestle, shaken for one minute with DCM (20 mL), filtered, and the filter cake powdered as before to provide the title compound as an easily-handled beige powder (4.037 g, 82%). ¹H NMR (400 MHz, DMSO-d6) δ 12.77 (br s, 1H), 10.85 (br s, 1H), 8.33 (m, 2H), 7.81 (m, 2H), 7.72 (m, 2H), 7.57 (m, 2H), 3.52 (m, 4H), 1.04 (t, 6H).

b. 1-(4-Diethylamino-phenyl)-3-(1-thieno [3,2-d]pyrimidin-4-yl-pyrrolidin-3-yl)-urea

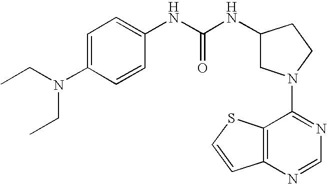

A solution of 1-thieno[3,2-d]pyrimidin-4-yl-pyrrolidin-3-ylamine hydrochloride (48 mg, 190 μmol), prepared as described in Example 13b, TEA (58 μL, 414 μmol), CHCl₃ (300 μL), and (4-diethylamino-phenyl)-carbamic acid 4-nitro-phenyl ester hydrochloride (77 mg, 210 μmol) were stirred at 80° C. for 20 min, then partitioned with DCM (2 mL) and 2.5M NaOH (2 mL). The aqueous layer was extracted with DCM (1×2 mL) and the organic layers were combined, dried (Na₂SO₄), and concentrated. Purification of the residue with C18 HPLC, followed by silica flash cartridge chromatography (EtOAc eluent) afforded the title compound (14.7 mg, 19%). ¹H NMR (400 MHz, CDCl₃) δ 8.46 (s, 1H), 7.72 (d, 1H), 7.38 (d, 1H), 7.05 (br m, 2H), 6.61 (br m, 2H), 6.19 (br s, 1H), 5.10 (br s, 1H), 4.61 (br s, 1H), 4.13 (m, 1H), 3.91 (m, 2H), 3.71 (m, 1H), 3.32 (brm, 4H), 2.31 (m, 1H), 2.03 (m, 1H), 1.13 (t, 6H). LC/MS (ESI): calcd mass 410.2, found 411.1 (MH)⁺.

### EXAMPLE 19

1-(4-Pyrrolidin-1-yl-phenyl)-3-(1-thieno [3,2-d]pyrimidin-4-yl-pyrrolidin-3-yl)-urea

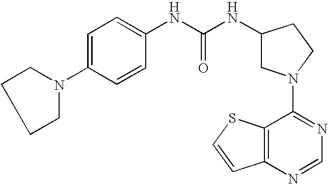

a. (4-Pyrrolidin-1-yl-phenyl)-carbamic acid 4-nitro-phenyl ester hydrochloride

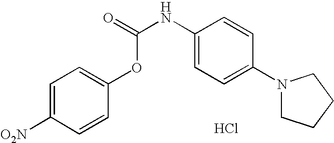

To a stirred solution of 4.9 g (30.4 mmol) of 4-pyrrolidin-1-yl-phenylamine in 70 mL of anhydrous THF at room temperature, was added dropwise a solution of 6.4 g (32 mmol) of 4-nitrophenyl chloroformate in 16 mL of anhydrous THF. After the addition was complete, the mixture was stirred for 1 h and then filtered. The precipitate was washed first with anhydrous THF (2×10 mL) and then with anhydrous DCM (3×10 mL) and dried in vacuo to yield 10 g of an off-white solid. ¹H-NMR (300 MHz, CD₃OD): 10.39 (s, 1H), 8.32 (d, 2H), 7.73 (d, 2H), 7.60 (d, 2H), 7.48 (d, 2H), 3.86-3.68 (bs, 4H), 2.35-2.24 (bs, 4H). LC/MS (ESI): 328 (MH)⁺.

b. 1-(4-Pyrrolidin-1-yl-phenyl)-3-(1-thieno [3,2-d]pyrimidin-4-yl-pyrrolidin-3-yl)-urea

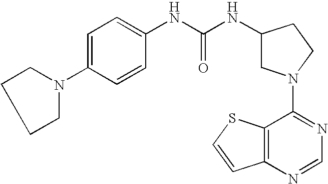

Prepared essentially as described in Example 18 using (4-pyrrolidin-1-yl-phenyl)-carbamic acid 4-nitro-phenyl ester hydrochloride, as described in the previous step, in place of (4-diethylamino-phenyl)-carbamic acid 4-nitro-phenyl ester hydrochloride. Purification was as follows: The combined organic layers were filtered and the filter cake was washed with DCM (1×2 mL) to afford the title compound as a powder (11 mg; 14%). ¹H NMR (400 MHz, CDCl₃) δ 8.40 (s, 1H), 8.20 (d, 1H), 7.90 (br s, 1H), 7.40 (d, 1H), 7.15 (m, 2H), 6.44 (m, 2H), 6.41 (br s, 1H), 4.33 (m, 1H), 4.15-3.80 (br m, 3H), 3.74 (br m, 1H), 3.15 (m, 4H), 2.23 (m, 1H), 1.96 (m, 1H), 1.91 (m, 4H). LC/MS (ESI): calcd mass 408.2, found 409.1 (MH)⁺.

### EXAMPLE 20

### 1-(6-Cyclobutoxy-pyridin-3-yl)-3-(1-thieno [3,2-d]pyrimidin-4-yl-piperidin-4-yl)-urea

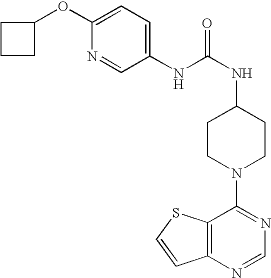

a.(1-Thieno [3,2-d]pyrimidin-4-yl-piperidin-4-yl)-carbamic acid tert-butyl ester

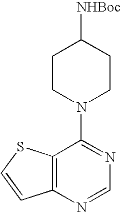

A solution of 4-chlorothieno[3,2-d]pyimidine (400 mg, 2.35 mmol), Piperidin-4-yl-carbamic acid tert-butyl ester (470 mg, 2.35 mmol), diisopropylethylamine (285 mg, 2.82 mmol) in isopropanol (10 mL) was heated to 100° C. for 2 hr. The resulting mixture was cooled to RT, poured into ethyl acetate (50 mL), and washed with water (25 mL). The organic layer was dried over anhydrous sodium sulfate, concentrated, and purified by silica gel chromatography (3% MeOH/EtOAc) to provide the title compound (672 mg, 86% yield). ¹H NMR (400 MHz, CD₃OD) δ 8.34 (s, 1H), 8.02 (d, 1H), 7.36 (d, 1H), 4.76 (m, 2H), 3.72 (m, 1H), 3.38 (m, 2H), 2.02 (m, 2H), 1.58-1.42 (m, 2H), 1.42 (s, 9H). LC/MS (ESI): calcd mass 334.2, found 335.2 (MH)⁺.

b. 1-Thieno[3,2-d]pyrimidin-4-yl-piperidin-4-ylamine

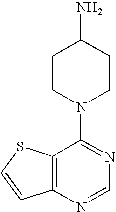

A solution of (1-Thieno[3,2-d]pyrimidin-4-yl-piperidin-4-yl)-carbamic acid tert-butyl ester (672 mg, 2.01 mmol), TFA (5 mL) and CH₂Cl₂ (10 mL) was stirred at RT for 16 h. The reaction mixture was concentrated then diluted with CH₂Cl₂ (100 mL) and sequentially washed with IN NaOH (50 mL) and brine (50 mL). The organic layer was dried over anhydrous sodium sulfate and concentrated to provide the title compound as an oil (290 mg, 62%). ¹H NMR (400 MHz, CDCl₃) δ 8.58 (s, 1H), 7.72 (d, 1H), 7.42 (d, 1H), 4.74 (m, 2H), 3.23 (m, 2H), 3.02 (m, 1H), 2.02 (m, 2H), 1.42 (m, 4H), 1.42 (s, 9H). LC/MS (ESI): calcd mass 234. 1, found 235.1 (MH)⁺.

c. 1-(6-Cyclobutoxy-pyridin-3-yl)-3-(1-thieno [3 ,2-d]pyrimidin-4-yl-piperidin-4-yl)-urea

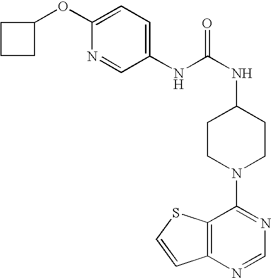

Prepared essentially as described in Example 18b using 1-thieno[3,2-d]pyrimidin-4-yl-piperidin-4-ylamine, prepared as described in the previous step, in place of 1-thieno[3,2-d]pyrimidin-4-yl-pyrrolidin-3-ylamine hydrochloride, and using (6-cyclobutoxy-pyridin-3-yl)-carbamic acid 4-nitro-phenyl ester (Example 15c) in place of (4-diethylamino-phenyl)-carbamic acid 4-nitro-phenyl ester hydrochloride. Also, 600 μL 95:5 CHCl₃/MeOH was used in place of 300 μL CHCl₃ to improve the solubility of the reaction components. Purification was as follows: The crude reaction was diluted with DCM (2 mL) and filtered. The filter cake was washed with DCM (1×2 mL) and dried to afford the title compound as a solid. ¹H NMR (400 MHz, DMSO-d6) δ 8.49 (s, 1H), 8.26 (br s, 1H), 8.21 (d, 1H), 8.07 (d, 1H), 7.74 (dd, 1H), 7.44 (d, 1H), 6.67 (d, 1H), 6.25 (d, 1H), 5.03 (p, 1H), 4.57 (m, 2H), 3.85 (m, 1H), 3.40 (m, 2H), 2.35 (m, 2H), 2.06-1.93 (m, 4H), 1.75 (m, 1H), 1.61 (m, 1H), 1.45 (m, 2H). LC/MS (ESI): calcd mass 424.2, found 425.1 (MH)⁺.

### EXAMPLE 21

### 1-(4-Cyclohexyl-phenyl)-3-(1-thieno [3,2-d]pyrimidin-4-yl-piperidin-4-yl)-urea

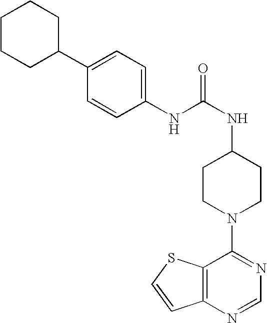

Prepared essentially as described in Example 18 using 1-thieno[3,2-d]pyrimidin-4-yl-piperidin-4-ylamine, prepared as described in Example 20b, in place of 1-thieno[3,2-d]pyrimidin-4-yl-pyrrolidin-3-ylamine hydrochloride, and using using (4-cyclohexyl-phenyl)-carbamic acid 4-nitro-phenyl ester (Example 16a) in place of (4-diethylamino-phenyl)-carbamic acid 4-nitro-phenyl ester hydrochloride. The title compound was purified as described in Example 20c. ¹H NMR (400 MHz, DMSO-d6) δ 8.49 (s, 1H), 8.24 (br s, 1H), 8.21 (d, 1H), 7.45 (d, 1H), 7.27 (m, 2H), 7.06 (m, 2H), 6.16 (d, 1H), 4.56 (m, 2H), 3.85 (m, 1H), 3.41 (m, 2H), 2.39 (m, 1H), 1.98 (m, 2H), 1.80-1.65 (m, 5H), 1.45 (m, 2H), 1.34 (m, 4H), 1.21 (m, 1H). LC/MS (ESI): calcd mass 435.2, found 436.1 (MH)⁺.

### EXAMPLE 22

### 1-(4-Phenoxy-phenyl)-3-(1-thieno [3,2-d]pyrimidin-4-yl-piperidin-4-yl)-urea

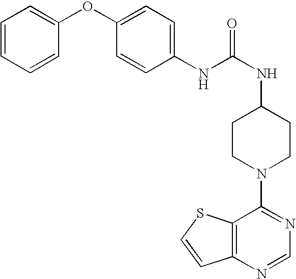

4-Phenoxyphenyl isocyanate (35 mg, 170 μmol) was added to a solution of 1-thieno[3,2-d]pyrimidin-4-yl-piperidin-4-ylamine (35 mg, 150 μmol) (Example 20b) in DCM (300 μL). The solution was stirred at rt overnight, at which point it became a slurry. The reaction was then partitioned with DCM (2 mL) and 2.0M K₂CO₃ (2 mL), the aqueous layer was extracted with 9:1 DCM/MeOH (2×2 mL), and the combined organic layers were filtered. The clear filtrate was dried (Na₂SO₄), concentrated, and purified by C18 HPLC followed by a bicarbonate solid phase extraction cartridge to afford the title compound (46.6 mg, 70%). ¹H NMR (400 MHz, CDCl₃) δ 8.57 (s, 1H), 7.72 (d, 1H), 7.42 (d, 1H), 7.33 (m, 2H), 7.22 (m, 2H), 7.10 (m, 1H), 6.97 (m, 4H), 6.19 (br s, 1H), 4.76 (m, 2H), 4.54 (d, 1H), 4.08 (m, 1H), 3.30 (m, 2H), 2.16 (m, 2H), 1.45 (m, 2H). LC/MS (ESI): calcd mass 445.2, found 446.1 (MH)⁺.

### EXAMPLE 23

### 1-(4-Pyrrolidin-1-yl-phenyl)-3-(1-thieno [3,2-d]pyrimidin-4-yl-piperidin-4-yl)-urea

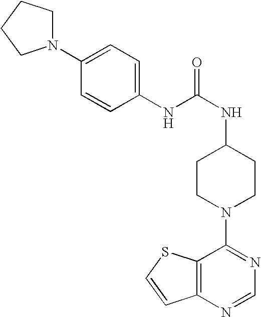

Prepared essentially as described in Example 18 using 1-thieno[3,2-d]pyrimidin-4-yl-piperidin-4-ylamine (Example 20b) instead of 1-thieno[3,2-d]pyrimidin-4-yl-pyrrolidin-3-ylamine hydrochloride, and using (4-pyrrolidin-1-yl-phenyl)-carbamic acid 4-nitro-phenyl ester hydrochloride (Example 19a) instead of (4-diethylamino-phenyl)-carbamic acid 4-nitro-phenyl ester hydrochloride. In addition, the reaction solvent was DMSO-d6 (300 μL) instead of CHCl₃ (300 μL). ¹H NMR (400 MHz, CDCl₃) δ 8.55 (s, 1H), 7.71 (d, 1H), 7.41 (d, 1H), 7.03 (m, 2H), 6.49 (m, 2H), 5.85 (br s, 1H), 4.70 (m, 2H), 4.40 (d, 1H), 4.05 (m, 1H), 3.32-3.22 (m, 6H), 2.10 (m, 2H), 2.00 (m, 4H), 1.37 (m, 2H). LC/MS (ESI): calcd mass 422.2, found 423.1 (MH)⁺.

### EXAMPLE 24

### 1-(4-Morpholin-4-yl-phenyl)-3-(1-thieno[3,2-d]pyrimidin-4-yl-piperidin-4-yl)-urea

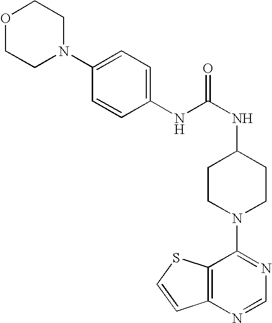

Prepared essentially as described in Example 20c, except (4-morpholin-4-yl-phenyl)-carbamic acid 4-nitro-phenyl ester hydrochloride (Example 14a) used in place of (6-cyclobutoxy-pyridin-3-yl)-carbamic acid 4-nitro-phenyl ester. ¹H NMR (400 MHz, CDCl₃) δ 8.56 (s, 1H), 7.71 (d, 1H), 7.41 (d, 1H), 7.13 (m, 2H), 6.86 (m, 2H), 6.07 (br s, 1H), 4.73 (m, 2H), 4.51 (d, 1H), 4.06 (m, 1H), 3.85 (m, 4H), 3.28 (m, 2H), 3.12 (m, 4H), 2.13 (m, 2H), 1.41 (m, 2H). LC/MS (ESI): calcd mass 438.2, found 439.1 (MH)⁺.

### EXAMPLE 25

### (6-Cyclobutoxy-pyridin-3-yl)-carbamic acid 1-thieno[3,2-d]pyrimidin-4-yl-pyrrolidin-3-yl ester

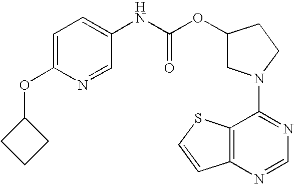

a. 1-Thieno[3,2-d]pyrimidin-4-yl-pyrrolidin-3-ol

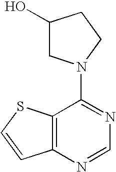

4-Chloro-thieno[3,2-d]pyrimidine (0.985 g, 5.78 mmol) was added to a mixture of racemic 3-pyrrolidinol (0.527 g, 6.06 mmol), DIPEA (1.10 mL, 6.31 mmol), and DMSO (1.5 mL). The mixture was stirred at “rt” for 2 min, during which time it spontaneously warmed and became a nearly homogeneous solution. The reaction was then stirred at 100° C. for 10 min, and the resulting homogeneous dark reddish-brown solution was allowed to cool to rt and then shaken with water (˜17 mL) before extracting with EtOAc (1×20 mL). The organic layer was washed with 4M NaCl (1×20 mL), dried (Na₂SO₄), and concentrated to give ˜150 mg of title compound. Additional title compound was obtained as follows: The aqueous layers were combined (˜40 mL) and extracted with EtOAc (1×300 mL), and the organic layer was dried (Na₂SO₄) and concentrated to give a powder. The two organic extract-derived powders were combined to give 911 mg of the title compound (71%). ¹H NMR (400 MHz, DMSO-d6) δ 8.38 (s, 1H), 8.18 (d, 1H), 7.39 (d, 1H), 5.12 (br s, 1H), 4.43 (br s, 1H), 4.05-3.68 (br m, 4H), 2.12-1.90 (m, 2H).

b. (6-Cyclobutoxy-pyridin-3-yl)-carbamic acid 1-thieno[3,2-d]pyrimidin-4-yl-pyrrolidin-3-yl ester

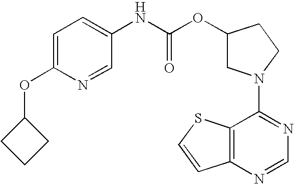

(6-Cyclobutoxy-pyridin-3-yl)-carbamic acid 4-nitro-phenyl ester (79 mg, 240 μmol) (Example 15c) was added to a homogeneous rt solution of 1-thieno[3,2-d]pyrimidin-4-yl-pyrrolidin-3-ol (44 mg, 200 μmol), as prepared in the previous step, DIPEA (108 μL, 620 μmol), and DMSO (200 μL). The resulting mixture was stirred at 100° C. for 20 min to give a homogeneous solution that was allowed to cool to rt. The reaction was then partitioned with 2M K₂CO₃ (2 mL) and DCM (2 mL), the aqueous layer was extracted with DCM (1×2 mL), and the combined organic layers were dried (Na₂SO₄) and concentrated. The residue was purified by C18 HPLC followed by solid phase extraction through a bicarbonate cartridge to afford the title compound (23.0 mg, 28%). ¹H NMR (400 MHz, CDCl₃) δ 8.53 (s, 1H), 8.04 (s, 1H), 7.78 (d, 1H), 7.74 (d, 1H), 7.41 (d, 1H), 6.83 (br s, 1H), 6.68 (d, 1H), 5.52 (m, 1H), 5.10 (p, 1H), 4.16 (m, 2H), 4.11-3.91 (m, 2H), 2.49-2.24 (m, 3H), 2.11 (m, 2H), 1.82 (m, 1H), 1.65 (m, 2H). LC/MS (ESI): calcd mass 411.1, found 412.1 (MH)⁺.

### EXAMPLE 26

### (4-Phenoxy-phenyl)-carbamic acid 1-thieno[3,2-d]pyrimidin-4-yl-pyrrolidin-3-yl ester

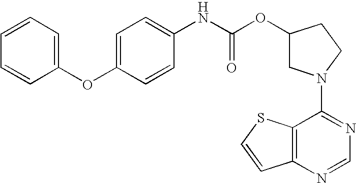

Prepared essentially as described for Example 25, except 4-phenoxyphenyl isocyanate was used in place of (6-cyclobutoxy-pyridin-3-yl)-carbamic acid 4-nitro-phenyl ester,

1.1 eq DIPEA (38 μL) was used instead of 3.1 eq DIPEA, and the reaction was stirred at rt for 1 hr before stirring at 100° C. for 20 min. ¹H NMR (400 MHz, CDCl₃) δ 8.54 (s, 1H), 7.75 (d, 1H), 7.41 (d, 1H), 7.38-7.29 (m, 4H), 7.08 (m, 1H), 6.98 (m, 4H), 6.81 (br s, 1H), 5.54 (m, 1H), 4.17 (m, 2H), 4.04 (m, 2H), 2.35 (m, 2H). LC/MS (ESI): calcd mass 432.1, found 433.1 (MH)⁺.

### EXAMPLE 27

### (4-Pyrrolidin-1-yl-phenyl)-carbamic acid 1-thieno[3,2-d]pyrimidin-4-yl-pyrrolidin-3-yl ester

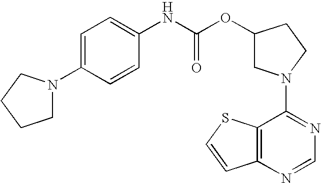

Prepared essentially as described for Example 25, using (4-pyrrolidin-1-yl-phenyl)-carbamic acid 4-nitro-phenyl ester hydrochloride (Example 19a) in place of (6-cyclobutoxy-pyridin-3-yl)-carbamic acid 4-nitro-phenyl ester. ¹H NMR (400 MHz, CDCl₃) δ 8.55 (s, 1H), 7.75 (d, 1H), 7.41 (d, 1H), 7.20 (m, 2H), 6.51 (m, 2H), 6.37 (br s, 1H), 5.52 (m, 1H), 4.21-3.95 (m, 4H), 3.25 (m, 4H), 2.32 (m, 2H), 1.99 (m, 4H). LC/MS (ESI): calcd mass 409.2, found 410.1 (MH)⁺.

### EXAMPLE 28

### (4-Morpholin-4-yl-phenyl)-carbamic acid 1-thieno[3,2-d]pyrimidin-4-yl-pyrrolidin-3-yl ester

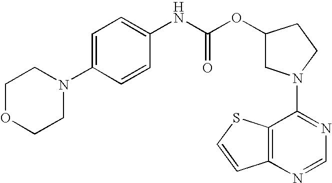

Prepared essentially as described for Example 25, using (4-morpholin-4-yl-phenyl)-carbamic acid 4-nitro-phenyl ester hydrochloride (Example 14a) in place of (6-cyclobutoxy-pyridin-3-yl)-carbamic acid 4-nitro-phenyl ester. ¹H NMR (400 MHz, CDCl₃) δ 8.54 (s, 1H), 7.75 (d, 1H), 7.41 (d, 1H), 7.28 (m, 2H), 6.87 (m, 2H), 6.63 (br s, 1H), 5.52 (m, 1H), 4.21-3.93 (m, 4H), 3.85 (m, 4H), 3.10 (m, 4H), 2.41-2.24 (m, 2H). LC/MS (ESI): calcd mass 425.1, found 426.1 (MH)⁺.

### EXAMPLE 29

### (4-Diethylamino-phenyl)-carbamic acid 1-thieno[3,2-d]pyrimidin-4-yl-pyrrolidin-3-yl ester

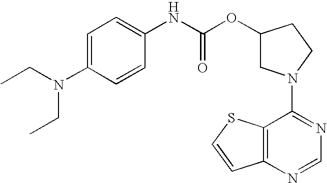

Prepared essentially as described for Example 25, using (4-diethylamino-phenyl)-carbamic acid 4-nitro-phenyl ester hydrochloride (Example 18a) in place of (6-cyclobutoxy-pyridin-3-yl)-carbamic acid 4-nitro-phenyl ester. ¹H NMR (400 MHz, CDCl₃) δ 8.53 (s, 1H), 7.73 (d, 1H), 7.40 (d, 1H), 7.19 (m, 2H), 6.63 (m, 3H), 5.51 (m, 1H), 4.19-3.90 (m, 4H), 3.31 (q, 4H), 2.40-2.21 (m, 2H), 1.12 (t, 6H). LC/MS (ESI): calcd mass 411.2, found 412.2 (MH)⁺.

Biological Activity

The following representative assays were performed in determining the biological activities of compounds within the scope of the invention. They are given to illustrate the invention in a non-limiting fashion.

Inhibition of FLT3 enzyme activity, MV4-11 proliferation and Baf3-FLT3 phosphorylation exemplify the specific inhibition of the FLT3 enzyme and cellular processes that are dependent on FLT3 activity. Inhibition of Baf3 cell proliferation is used as a test of FLT3 independent cytotoxicity of compounds within the scope of the invention. All of the examples herein show significant and specific inhibition of the FLT3 kinase and FLT3-dependent cellular responses. The compounds of the present invention are also cell permeable.

FLT3 Fluorescence Polarization Kinase Assay

The FLT3 FP assay utilizes the fluorescein-labeled phosphopeptide and the anti-phosphotyrosine antibody included in the Panvera Phospho-Tyrosine Kinase Kit (Green) supplied by Invitrogen. When FLT3 phosphorylates poly Glu₄Tyr, the fluorescein-labeled phosphopeptide is displaced from the anti-phosphotyrosine antibody by the phosphorylated poly Glu4Tyr, thus decreasing the FP value. The FLT3 kinase reaction is incubated at room temperature for 30 minutes under the following conditions: 10 nM FLT3 571-993, 20 ug/mL poly Glu4Tyr, 150 uM ATP, 5mM MgCl₂, 1% compound in DMSO. The kinase reaction is stopped with the addition of EDTA. The fluorescein-labeled phosphopeptide and the anti-phosphotyrosine antibody are added and incubated for 30 minutes at room temperature.

All data points are an average of triplicate samples. Inhibition and IC₅₀ data analysis was done with GraphPad Prism using a non-linear regression fit with a multiparamater, sigmoidal dose-response (variable slope) equation. The IC₅₀ for kinase inhibition represents the dose of a compound that results in a 50% inhibition of kinase activity compared to DMSO vehicle control.

Growth Inhibition Of MV4-11 And Baf3 Cells

FLT3 specific growth inhibition was measured in the leukemic cell line MV4-11 (ATCC Number: CRL-9591). MV4-11 cells are derived from a patient with childhood acute myelomonocytic leukemia with an 11 q23 translocation resulting in a MLL gene rearrangement and containing an FLT3-ITD mutation (AML subtype M4)(1,2). MV4-11 cells cannot grow and survive without active FLT31TD.

The IL-3 dependent, murine b-cell lymphoma cell line, Baf3, were used as a control to confirm the selectivity of the compounds of the present invention by measuring non-specific growth inhibition by the compounds of the present invention.

To measure proliferation inhibition by test compounds the luciferase based CellTiterGlo reagent (Promega) was used. Cells are plated at 10,000 cells per well in 100ul of in RPMI media containing penn/strep, 10% FBS and lng/ml GM-CSF or 1 ng/ml IL-3 for MV4-11 and Baf3 cells respectively.

Compound dilutions or 0.1% DMSO (vehicle control) are added to cells and the cells are allowed to grow for 72 hours at standard cell growth conditions (37° C., 5% CO₂). Total cell growth is quantified as the difference in luminescent counts (relative light units, RLU) of cell number at Day 0 compared to total cell number at Day 3 (72 hours of growth and/or compound treatment). One hundred percent inhibition of growth is defined as an RLU equivalent to the Day 0 reading. Zero percent inhibition is defined as the RLU signal for the DMSO vehicle control at Day 3 of growth. All data points are an average of triplicate samples. The IC₅₀ for growth inhibition represents the dose of a compound that results in a 50% inhibition of total cell growth at day 3 of the DMSO vehicle control. Inhibition and IC₅₀ data analysis was done with GraphPad Prism using a non-linear regression fit with a multiparamater, sigmoidal dose-response (variable slope) equation.

MV-411 cells expressed the FLT3 internal tandem duplication mutation, and thus were entirely dependent upon FLT3 activity for growth. Strong activity against the MV4-11 cells is anticipated to be a desirable quality of the invention. In contrast, the Baf3 cell proliferations is driven by the cytokine IL-3 and these cells are used as a non-specific toxicity control for test compounds. All compounds examples in the present invention showed <50% inhibition at a 3uM dose (data is not included), suggesting that the compounds are not cytotoxic and have good selectivity for FLT3.

Cell-Based FLT3 Receptor Elisa

Cells overexpressing the FLT3 receptor were obtained from Dr. Michael Heinrich (Oregon Health and Sciences University). The Baf3 FLT3 cell lines were created by stable transfection of parental Baf3 cells (a murine B cell lymphoma line dependent on the cytokine IL-3 for growth) with wild-type FLT3. Cells were selected for their ability to grow in the absence of IL-3 and in the presence of FLT3 ligand.

Baf3 cells were maintained in RPMI 1640 with 10%FCS, penn/strep and 10 ng/mL FLT ligand at 37° C., 5% CO₂. To measure direct inhibition of the wild-type FLT3 receptor activity and phosphorylation a sandwich ELISA method was developed similar to those developed for other RTKs (3,4). 200ul of Baf3FLT3 cells (1×10⁶/ml) were plated in 96 well dishes in RPM11640 with 0.5% serum and 0.01ng/ml IL-3 for 16 hours prior to 1 hour compound or DMSO vehicle incubation. Cells were treated with 100 ng/ml Flt ligand (R&D Systems Cat# 308-FK) for 10 min. at 37° C. Cells were pelleted, washed and lysed in 100ul HNTG buffer (50 mM Hepes, 150 mM NaCl, 10% Glycerol, 1% Triton -X-100, 10 mM NaF, 1 mM EDTA, 1.5 mM MgCl₂, 10 mM NaPyrophosphate) supplemented with phosphatase (Sigma Cat# P2850) and protease inhibitors (Sigma Cat #P8340). Lysates were cleared by centrifugation at 1000×g for 5 minutes at 4° C. Cell lysates were transferred to white wall 96 well microtiter (Costar #9018) plates coated with 50ng/well anti-FLT3 antibody (Santa Cruz Cat# sc-480) and blocked with SeaBlock reagent (Pierce Cat#37527). Lysates were incubated at 4° C. for 2 hours. Plates were washed 3× with 200ul/well PBS/0.1% triton-X-100. Plates are then incubated with 1:8000 dilution of HRP-conjugated anti-phosphotyrosine antibody (Clone 4G10, Upstate Biotechnology Cat#16-105) for 1 hour at room temperature. Plates were washed 3× with 200ul/well PBS/0.1% triton-X-100. Signal detection with Super Signal Pico reagent (Pierce Cat#37070) was done according to manufacturer's instruction with a Berthold microplate luminometer. All data points are an average of triplicate samples. The total relative light units (RLU) of Flt ligand stimulated FLT3 phosphorylation in the presence of 0.1% DMSO control was defined as 0% inhibition and 100% inhibition was the total RLU of lysate in the basal state. Inhibition and IC₅₀ data analysis was done with GraphPad Prism using a non-linear regression fit with a multiparamater, sigmoidal dose-response (variable slope) equation.

Biological Procedure References

1. Drexler H G. The Leukemia-Lymphoma Cell Line Factsbook. Academic Pres: San Diego, Calif., 2000.

2. Quentmeier H, Reinhardt J, Zaborski M, Drexler H G. FLT3 mutations in acute myeloid leukemia cell lines. Leukemia. 2003 January; 17:120-124.

3. Sadick, M D, Sliwkowski, M X, Nuijens, A, Bald, L, Chiang, N, Lofgren, J A, Wong W L T. Analysis of Heregulin-Induced ErbB2 Phosphorylation with a High-Throughput Kinase Receptor Activation Enzyme-Linked Immunsorbent Assay, Analytical Biochemistry. 1996; 235:207-214.

4. Baumann C A, Zeng L, Donatelli R R, Maroney A C. Development of a quantitative, high-throughput cell-based enzyme-linked immunosorbent assay for detection of colony-stimulating factor-1 receptor tyrosine kinase inhibitors. J Biochem Biophys Methods. 2004; 60:69-79.

Biological Data

Biological Data for FLT3

The activity of representative compounds of the present invention is presented in the charts below. All activities are in μM and have the following uncertainties: FLT3 kinase: +10%; MV4-11 and Baf3-FLT3: +20%.

| | | FLT3 | | BaF3 |
|---|---|---|---|---|
| | | Kinase | MV4-11 | ELISA |
| No. | Compound Name | (μM) | (μM) | (μM) |
| 1 | (4-Isopropyl-phenyl)-carbamic acid 1-thieno[2,3- | 0.102 | 7.200 | nd |
| | d]pyrimidin-4-yl-piperidin-4-yl ester | | | |
| 2 | (4-Isopropoxy-phenyl)-carbamic acid 1-thieno[2,3- | 2.09 | >10 | nd |
| | d]pyrimidin-4-yl-piperidin-4-yl ester | | | |
| 3 | (4-Isopropyl-phenyl)-carbamic acid 1-thieno[2,3- | 0.227 | 2.4 | nd |
| | d]pyrimidin-4-yl-pyrrolidin-3-yl ester | | | |
| 4 | (4-Isopropoxy-phenyl)-carbamic acid 1-thieno[2,3- | 0.761 | >10 | nd |
| | d]pyrimidin-4-yl-pyrrolidin-3-yl ester | | | |
| 5 | (4-Isopropyl-phenyl)-carbamic acid 1-thieno[3,2- | 0.064 | 1.3 | 0.022 |
| | d]pyrimidin-4-yl-pyrrolidin-3-yl ester | | | |
| 6 | (4-Isopropoxy-phenyl)-carbamic acid 1-thieno[3,2- | 0.208 | 6.5 | nd |
| | d]pyrimidin-4-yl-pyrrolidin-3-yl ester | | | |
| 7 | (4-Isopropyl-phenyl)-carbamic acid 1-thieno[3,2- | 0.14 | 4.3 | nd |
| | d]pyrimidin-4-yl-piperidin-4-yl ester | | | |
| 8 | (4-Isopropoxy-phenyl)-carbamic acid 1-thieno[3,2- | 3.03 | 3.6 | nd |
| | d]pyrimidin-4-yl-piperidin-4-yl ester | | | |
| 9 | 1-(4-Isopropyl-phenyl)-3-(1-thieno[2,3- | 0.041 | 1.3 | 0.055 |
| | d]pyrimidin-4-yl-pyrrolidin-3-yl)-urea | | | |
| 10 | 1-(4-Isopropoxy-phenyl)-3-(1-thieno[2,3- | 0.365 | 3.6 | nd |
| | d]pyrimidin-4-yl-pyrrolidin-3-yl)-urea | | | |
| 11 | 1-(4-Isopropyl-phenyl)-3-(1-thieno[3,2- | 0.077 | 0.671 | 0.108 |
| | d]pyrimidin-4-yl-pyrrolidin-3-yl)-urea | | | |
| 12 | 1-(4-Isopropoxy-phenyl)-3-(1-thieno[3,2- | 0.296 | 2.5 | nd |
| | d]pyrimidin-4-yl-pyrrolidin-3-yl)-urea | | | |
| 13 | 1-(4-Phenoxy-phenyl)-3-(1-thieno[3,2-d]pyrimidin- | nd | 0.881 | >5 |
| | 4-yl-pyrrolidin-3-yl)-urea | | | |
| 14 | 1-(4-Morpholin-4-yl-phenyl)-3-(1-thieno[3,2- | nd | >5 | nd |
| | d]pyrimidin-4-yl-pyrrolidin-3-yl)-urea | | | |
| 15 | 1-(6-Cyclobutoxy-pyridin-3-yl)-3-(1-thieno[3,2- | nd | 0.983 | 1.9 |
| | d]pyrimidin-4-yl-pyrrolidin-3-yl)-urea | | | |
| 16 | 1-(4-Cyclohexyl-phenyl)-3-(1-thieno[3,2- | nd | 5.2 | nd |
| | d]pyrimidin-4-yl-pyrrolidin-3-yl)-urea | | | |
| 17 | 1-(4-Bromo-phenyl)-3-(1-thieno[3,2-d]pyrimidin- | nd | 1.9 | nd |
| | 4-yl-pyrrolidin-3-yl)-urea | | | |
| 18 | 1-(4-Diethylamino-phenyl)-3-(1-thieno[3,2- | nd | 0.757 | 0.895 |
| | d]pyrimidin-4-yl-pyrrolidin-3-yl)-urea | | | |
| 19 | 1-(4-Pyrrolidin-1-yl-phenyl)-3-(1-thieno[3,2- | nd | 0.85 | 2.9 |
| | d]pyrimidin-4-yl-pyrrolidin-3-yl)-urea | | | |
| 20 | 1-(6-Cyclobutoxy-pyridin-3-yl)-3-(1-thieno[3,2- | nd | 1.5 | nd |
| | d]pyrimidin-4-yl-piperidin-4-yl)-urea | | | |
| 21 | 1-(4-Cyclohexyl-phenyl)-3-(1-thieno[3,2- | nd | 1.4 | nd |
| | d]pyrimidin-4-yl-piperidin-4-yl)-urea | | | |
| 22 | 1-(4-Phenoxy-phenyl)-3-(1-thieno[3,2-d]pyrimidin- | nd | 3.8 | nd |
| | 4-yl-piperidin-4-yl)-urea | | | |
| 23 | 1-(4-Pyrrolidin-1-yl-phenyl)-3-(1-thieno[3,2- | nd | 3.9 | nd |
| | d]pyrimidin-4-yl-piperidin-4-yl)-urea | | | |
| 24 | 1-(4-Morpholin-4-yl-phenyl)-3-(1-thieno[3,2- | nd | >5 | nd |
| | d]pyrimidin-4-yl-piperidin-4-yl)-urea | | | |
| 25 | (6-Cyclobutoxy-pyridin-3-yl)-carbamic acid 1- | nd | 0.345 | 1.6 |
| | thieno[3,2-d]pyrimidin-4-yl-pyrrolidin-3-yl ester | | | |
| 26 | (4-Phenoxy-phenyl)-carbamic acid 1-thieno[3,2- | nd | 0.953 | 0.39 |
| | d]pyrimidin-4-yl-pyrrolidin-3-yl ester | | | |
| 27 | (4-Pyrrolidin-1-yl-phenyl)-carbamic acid 1- | nd | nd | nd |
| | thieno[3,2-d]pyrimidin-4-yl-pyrrolidin-3-yl ester | | | |
| 28 | (4-Morpholin-4-yl-phenyl)-carbamic acid 1- | nd | 2.1 | nd |
| | thieno[3,2-d]pyrimidin-4-yl-pyrrolidin-3-yl ester | | | |
| 29 | (4-Diethylamino-phenyl)-carbamic acid 1- | nd | nd | nd |
| | thieno[3,2-d]pyrimidin-4-yl-pyrrolidin-3-yl ester | | | |

Methods of Treatment Prevention

In another aspect of this invention, compounds of the invention can be used to inhibit tyrosine kinase activity, including Flt3 activity, or reduce kinase activity, including Flt3 activity, in a cell or a subject, or to treat disorders related to FLT3 kinase activity or expression in a subject.

In one embodiment to this aspect, the present invention provides a method for reducing or inhibiting the kinase activity of FLT3 in a cell comprising the step of contacting the cell with a compound of Formula I or Formula II. The present invention also provides a method for reducing or inhibiting the kinase activity of FLT3 in a subject comprising the step of administering a compound of Formula I or Formula II to the subject. The present invention further provides a method of inhibiting cell proliferation in a cell comprising the step of contacting the cell with a compound of Formula I or Formula II.

The kinase activity of FLT3 in a cell or a subject can be determined by procedures well known in the art, such as the FLT3 kinase assay described herein.

The term “subject” as used herein, refers to an animal, preferably a mammal, most preferably a human, who has been the object of treatment, observation or experiment.

The term “contacting” as used herein, refers to the addition of compound to cells such that compound is taken up by the cell.

In other embodiments to this aspect, the present invention provides both prophylactic and therapeutic methods for treating a subject at risk of (or susceptible to) developing a cell proliferative disorder or a disorder related to FLT3.

In one example, the invention provides methods for preventing in a subject a cell proliferative disorder or a disorder related to FLT3, comprising administering to the subject a prophylactically effective amount of a pharmaceutical composition comprising the compound of Formula I or Formula II and a pharmaceutically acceptable carrier. Administration of said prophylactic agent can occur prior to the manifestation of symptoms characteristic of the cell proliferative disorder or disorder related to FLT3, such that a disease or disorder is prevented or, alternatively, delayed in its progression.

In another example, the invention pertains to methods of treating in a subject a cell proliferative disorder or a disorder related to FLT3 comprising administering to the subject a therapeutically effective amount of a pharmaceutical composition comprising the compound of Formula I or Formula II and a pharmaceutically acceptable carrier. Administration of said therapeutic agent can occur concurrently with the manifestation of symptoms characteristic of the disorder, such that said therapeutic agent serves as a therapy to compensate for the cell proliferative disorder or disorders related to FLT3.

The term “prophylactically effective amount” refers to an amount of an active compound or pharmaceutical agent that inhibits or delays in a subject the onset of a disorder as being sought by a researcher, veterinarian, medical doctor or other clinician.

The term “therapeutically effective amount” as used herein, refers to an amount of active compound or pharmaceutical agent that elicits the biological or medicinal response in a subject that is being sought by a researcher, veterinarian, medical doctor or other clinician, which includes alleviation of the symptoms of the disease or disorder being treated.

Methods are known in the art for determining therapeutically and prophylactically effective doses for the instant pharmaceutical composition.

As used herein, the term “composition” is intended to encompass a product comprising the specified ingredients in the specified amounts, as well as any product which results, directly or indirectly, from combinations of the specified ingredients in the specified amounts.

As used herein, the terms “disorders related to FLT3”, or “disorders related to FLT3 receptor”, or “disorders related to FLT3 receptor tyrosine kinase” shall include diseases associated with or implicating FLT3 activity, for example, the overactivity of FLT3, and conditions that accompany with these diseases. The term “overactivity of FLT3” refers to either 1) FLT3 expression in cells which normally do not express FLT3; 2) FLT3 expression by cells which normally do not express FLT3; 3) increased FLT3 expression leading to unwanted cell proliferation; or 4) mutations leading to constitutive activation of FLT3. Examples of “disorders related to FLT3” include disorders resulting from over stimulation of FLT3 due to abnormally high amount of FLT3 or mutations in FLT3, or disorders resulting from abnormally high amount of FLT3 activity due to abnormally high amount of FLT3 or mutations in FLT3. It is known that overactivity of FLT3 has been implicated in the pathogenesis of a number of diseases, including the cell proliferative disorders, neoplastic disorders and cancers listed below.

The term “cell proliferative disorders” refers to unwanted cell proliferation of one or more subset of cells in a multicellular organism resulting in harm (i.e., discomfort or decreased life expectancy) to the multicellular organisms. Cell proliferative disorders can occur in different types of animals and humans. For example, as used herein “cell proliferative disorders” include neoplastic and other cell proliferative disorders.

As used herein, a “neoplastic disorder” refers to a tumor resulting from abnormal or uncontrolled cellular growth. Examples of neoplastic disorders include, but are not limited to, hematopoietic disorders such as, for instance, the myeloproliferative disorders, such as thrombocythemia, essential thrombocytosis (ET), agnogenic myeloid metaplasia, myelofibrosis (MF), myelofibrosis with myeloid metaplasia (MMM), chronic idiopathic myelofibrosis (1MF), and polycythemia vera (PV), the cytopenias, and pre-malignant myelodysplastic syndromes; cancers such as glioma cancers, lung cancers, breast cancers, colorectal cancers, prostate cancers, gastric cancers, esophageal cancers, colon cancers, pancreatic cancers, ovarian cancers, and hematoglogical malignancies, including myelodysplasia, multiple myeloma, leukemias and lymphomas. Examples of hematological malignancies include, for instance, leukemias, lymphomas (non-Hodgkin's lymphoma), Hodgkin's disease (also called Hodgkin's lymphoma), and myeloma—for instance, acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myelodysplastic syndromes (MDSs), myeloproliferative disorders (MPD), and multiple myeloma, (MM).

Examples of other cell proliferative disorders, include but are not limited to, atherosclerosis (Libby P, 2003, “Vascular biology of atherosclerosis: overview and state of the art”, Am J Cardiol 91(3A):3A-6A) transplantation-induced vasculopathies (Helisch A, Schaper W. 2003, Arteriogenesis: the development and growth of collateral arteries. Microcirculation, 10(1):83-97), macular degeneration (Holz FG et al., 2004, “Pathogenesis of lesions in late age-related macular disease”, Am J Ophthalmol. 137(3):504-10), neointima hyperplasia and restenosis (Schiele T M et. al., 2004, “Vascular restenosis—striving for therapy.” Expert Opin Pharmacother. 5(11):2221-32) , pulmonary fibrosis (Thannickal VJ et al., 2003, “Idiopathic pulmonary fibrosis: emerging concepts on pharmacotherapy, Expert Opin Pharmacother. 5(8): 1671-86), glomerulonephritis (Cybulsky A V, 2000, “Growth factor pathways in proliferative glomerulonephritis”, Curr Opin Nephrol Hypertens”9(3):217-23), glomerulosclerosis (Harris R C et al, 1999, “Molecular basis of injury and progression in focal glomerulosclerosis” Nephron 82(4):289-99), renal dysplasia and kidney fibrosis (Woolf A S et al., 2004, “Evolving concepts in human renal dysplasia”, J Am Soc Nephrol.15(4):998-1007), diabetic retinopathy (Grant M B et al., 2004, “The role of growth factors in the pathogenesis of diabetic retinopathy”, Expert Opin Investig Drugs 13(10):1275-93) and rheumatoid arthritis (Sweeney S E, Firestein G S, 2004, Rheumatoid arthritis: regulation of synovial inflammation, Int J Biochem Cell Biol. 36(3):372-8).

In a further embodiment to this aspect, the invention encompasses a combination therapy for treating or inhibiting the onset of a cell proliferative disorder or a disorder related to FLT3 in a subject. The combination therapy comprises administering to the subject a therapeutically or prophylactically effective amount of a compound of Formula I or Formula II, and one or more other anti-cell proliferation therapy including chemotherapy, radiation therapy, gene therapy and immunotherapy.

In an embodiment of the present invention, the compound of the present invention may be administered in combination with chemotherapy. As used herein, chemotherapy refers to a therapy involving a chemotherapeutic agent. A variety of chemotherapeutic agents may be used in the combined treatment methods disclosed herein. Chemotherapeutic agents contemplated as exemplary, include, but are not limited to: platinum compounds (e.g.,cisplatin, carboplatin, oxaliplatin); taxane compounds (e.g., paclitaxcel, docetaxol); campotothecin compounds (irinotecan, topotecan); ; vinca alkaloids (e.g., vincristine, vinblastine, vinorelbine); anti-tumor nucleoside derivatives (e.g., 5-fluorouracil, leucovorin, gemcitabine, capecitabine) alkylating agents (e.g., cyclophosphamide, carmustine, lomustine, thiotepa); epipodophyllotoxins /podophyllotoxins (e.g. etoposide, teniposide); aromatase inhibitors (e.g., anastrozole, letrozole, exemestane); anti-estrogen compounds (e.g., tamoxifen, fulvestrant), antifolates (e.g., premetrexed disodium); hypomethylating agents (e.g., azacitidine); biologics (e.g., gemtuzamab, cetuximab, rituximab, pertuzumab, trastuzumab, bevacizumab, erlotinib); antibiotics/anthracyclines (e.g. idarubicin, actinomycin D, bleomycin, daunorubicin, doxorubicin, mitomycin C, dactinomycin, carminomycin, daunomycin); antimetabolites (e.g., aminopterin, clofarabine, cytosine arabinoside, methotrexate); tubulin-binding agents (e.g. combretastatin, colchicine, nocodazole); topoisomerase inhibitors (e.g., camptothecin). Further useful agents include verapamil, a calcium antagonist found to be useful in combination with antineoplastic agents to establish chemosensitivity in tumor cells resistant to accepted chemotherapeutic agents and to potentiate the efficacy of such compounds in drug-sensitive malignancies. See Simpson W G, The calcium channel blocker verapamil and cancer chemotherapy. Cell Calcium. 1985 December;6(6):449-67. Additionally, yet to emerge chemotherapeutic agents are contemplated as being useful in combination with the compound of the present invention.

In another embodiment of the present invention, the compound of the present invention may be administered in combination with radiation therapy. As used herein, “radiation therapy” refers to a therapy comprising exposing the subject in need thereof to radiation. Such therapy is known to those skilled in the art. The appropriate scheme of radiation therapy will be similar to those already employed in clinical therapies wherein the radiation therapy is used alone or in combination with other chemotherapeutics.

In another embodiment of the present invention, the compound of the present invention may be administered in combination with a gene therapy. As used herein, “gene therapy” refers to a therapy targeting on particular genes involved in tumor development. Possible gene therapy strategies include the restoration of defective cancer-inhibitory genes, cell transduction or transfection with antisense DNA corresponding to genes coding for growth factors and their receptors, RNA-based strategies such as ribozymes, RNA decoys, antisense messenger RNAs and small interfering RNA (siRNA) molecules and the so-called ‘suicide genes’.

In other embodiments of this invention, the compound of the present invention may be administered in combination with an immunotherapy. As used herein, “immunotherapy” refers to a therapy targeting particular protein involved in tumor development via antibodies specific to such protein. For example, monoclonal antibodies against vascular endothelial growth factor have been used in treating cancers.

Where a second pharmaceutical is used in addition to a compound of the present invention, the two pharmaceuticals may be administered simultaneously (e.g. in separate or unitary compositions) sequentially in either order, at approximately the same time, or on separate dosing schedules. In the latter case, the two compounds will be administered within a period and in an amount and manner that is sufficient to ensure that an advantageous or synergistic effect is achieved. It will be appreciated that the preferred method and order of administration and the respective dosage amounts and regimes for each component of the combination will depend on the particular chemotherapeutic agent being administered in conjunction with the compound of the present invention, their route of administration, the particular tumor being treated and the particular host being treated.

As will be understood by those of ordinary skill in the art, the appropriate doses of chemotherapeutic agents will be generally similar to or less than those already employed in clinical therapies wherein the chemotherapeutics are administered alone or in combination with other chemotherapeutics.

The optimum method and order of administration and the dosage amounts and regime can be readily determined by those skilled in the art using conventional methods and in view of the information set out herein.

By way of example only, platinum compounds are advantageously administered in a dosage of 1 to 500 mg per square meter (mg/m²) of body surface area, for example 50 to 400 mg/m², particularly for cisplatin in a dosage of about 75 mg/m² and for carboplatin in about 30 mg/m² per course of treatment. Cisplatin is not absorbed orally and must therefore be delivered via injection intravenously, subcutaneously, intratumorally or intraperitoneally.

By way of example only, taxane compounds are advantageously administered in a dosage of 50 to 400 mg per square meter (mg/m²) of body surface area, for example 75 to 250 mg/m², particularly for paclitaxel in a dosage of about 175 to 250 mg/m² and for docetaxel in about 75 to 150 mg/m per course of treatment.

By way of example only, camptothecin compounds are advantageously administered in a dosage of 0.1 to 400 mg per square meter (mg/m²) of body surface area, for example 1 to 300 mg/m², particularly for irinotecan in a dosage of about 100 to 350 mg/m² and for topotecan in about 1 to 2 mg/m² per course of treatment.

By way of example only, vinca alkaloids may be advantageously administered in a dosage of 2 to 30 mg per square meter (mg/m²) of body surface area, particularly for vinblastine in a dosage of about 3 to 12 mg/m² , for vincristine in a dosage of about 1 to 2 mg/m², and for vinorelbine in dosage of about 10 to 30 mg/m² per course of treatment.

By way of example only, anti-tumor nucleoside derivatives may be advantageously administered in a dosage of 200 to 2500 mg per square meter (mg/m²) of body surface area, for example 700 to 1500 mg/m². 5-fluorouracil (5-FU) is commonly used via intravenous administration with doses ranging from 200 to 500 mg/m² (preferably from 3 to 15 mg/kg/day). Gemcitabine is advantageously administered in a dosage of about 800 to 1200 mg/m² and capecitabine is advantageously administered in about 1000 to 2500 mg/m² per course of treatment.

By way of example only, alkylating agents may be advantageously administered in a dosage of 100 to 500 mg per square meter (mg/m²) of body surface area, for example 120 to 200 mg/m², particularly for cyclophosphamide in a dosage of about 100 to 500 mg/m², for chlorambucil in a dosage of about 0.1 to 0.2 mg/kg of body weight, for carmustine in a dosage of about 150 to 200 mg/m², and for lomustine in a dosage of about 100 to 150 mg/m2 per course of treatment.

By way of example only, podophyllotoxin derivatives may be advantageously administered in a dosage of 30 to 300 mg per square meter (mg/m2) of body surface area, for example 50 to 250 mg/m², particularly for etoposide in a dosage of about 35 to 100 mg/m² and for teniposide in about 50 to 250 mg/m² per course of treatment.

By way of example only, anthracycline derivatives may be advantageously administered in a dosage of 10 to 75 mg per square meter (mg/m²) of body surface area, for example 15 to 60 mg/m², particularly for doxorubicin in a dosage of about 40 to 75 mg/m², for daunorubicin in a dosage of about 25 to 45 mg/m², and for idarubicin in a dosage of about 10 to 15 mg/m² per course of treatment.

By way of example only, anti-estrogen compounds may be advantageously administered in a dosage of about 1 to 100 mg daily depending on the particular agent and the condition being treated. Tamoxifen is advantageously administered orally in a dosage of 5 to 50 mg, preferably 10 to 20 mg twice a day, continuing the therapy for sufficient time to achieve and maintain a therapeutic effect. Toremifene is advantageously administered orally in a dosage of about 60 mg once a day, continuing the therapy for sufficient time to achieve and maintain a therapeutic effect. Anastrozole is advantageously administered orally in a dosage of about lmg once a day. Droloxifene is advantageously administered orally in a dosage of about 20-100 mg once a day. Raloxifene is advantageously administered orally in a dosage of about 60 mg once a day. Exemestane is advantageously administered orally in a dosage of about 25 mg once a day.

By way of example only, biologics may be advantageously administered in a dosage of about 1 to 5 mg per square meter (mg/m²) of body surface area, or as known in the art, if different. For example, trastuzumab is advantageously administered in a dosage of 1 to 5 mg/m² particularly 2 to 4 mg/m² per course of treatment.

Dosages may be administered, for example once, twice or more per course of treatment, which may be repeated for example every 7, 14, 21 or 28 days.

The compounds of the present invention can be administered to a subject systemically, for example, intravenously, orally, subcutaneously, intramuscular, intradermal, or parenterally. The compounds of the present invention can also be administered to a subject locally. Non-limiting examples of local delivery systems include the use of intraluminal medical devices that include intravascular drug delivery catheters, wires, pharmacological stents and endoluminal paving. The compounds of the present invention can further be administered to a subject in combination with a targeting agent to achieve high local concentration of the compound at the target site. In addition, the compounds of the present invention may be formulated for fast-release or slow-release with the objective of maintaining the drugs or agents in contact with target tissues for a period ranging from hours to weeks.

The present invention also provides a pharmaceutical composition comprising a compound of Formula I or Formula II in association with a pharmaceutically acceptable carrier. The pharmaceutical composition may contain between about 0.1 mg and 1000 mg, preferably about 100 to 500 mg, of the compound, and may be constituted into any form suitable for the mode of administration selected.

The phrases “pharmaceutically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

The pharmaceutical composition of the present invention also includes a pharmaceutical composition for slow release of a compound of the present invention. The composition includes a slow release carrier (typically, a polymeric carrier) and a compound of the present invention.

Slow release biodegradable carriers are well known in the art. These are materials that may form particles that capture therein an active compound(s) and slowly degrade/dissolve under a suitable environment (e.g., aqueous, acidic, basic, etc) and thereby degrade/dissolve in body fluids and release the active compound(s) therein. The particles are preferably nanoparticles (i.e., in the range of about 1 to 500 nm in diameter, preferably about 50-200 nm in diameter, and most preferably about 100 nm in diameter).

The present invention also provides methods to prepare the pharmaceutical compositions of this invention. The compound of Formula I or Formula II, as the active ingredient, is intimately admixed with a pharmaceutical carrier according to conventional pharmaceutical compounding techniques, which carrier may take a wide variety of forms depending on the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, though other ingredients, for example, for purposes such as aiding solubility or for preservation, may be included. Injectable suspensions may also be prepared, in which case appropriate liquid carriers, suspending agents and the like may be employed. In preparation for slow release, a slow release carrier, typically a polymeric carrier, and a compound of the present invention are first dissolved or dispersed in an organic solvent. The obtained organic solution is then added into an aqueous solution to obtain an oil-in-water-type emulsion. Preferably, the aqueous solution includes surface-active agent(s). Subsequently, the organic solvent is evaporated from the oil-in-water-type emulsion to obtain a colloidal suspension of particles containing the slow release carrier and the compound of the present invention.

The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, from about 0.01 mg to 200 mg/kg of body weight per day. Preferably, the range is from about 0.03 to about 100 mg/kg of body weight per day, most preferably, from about 0.05 to about 10 mg/kg of body weight per day. The compounds may be administered on a regimen of 1 to 5 times per day. The dosages, however, may be varied depending upon the requirement of the patients, the severity of the condition being treated and the compound being employed. The use of either daily administration or post-periodic dosing may be employed.

Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, acetyl alcohol and cellulose acetate.

The liquid forms in which the compound of Formula I or Formula II may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin. The liquid forms in suitably flavored suspending or dispersing agents may also include the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

Advantageously, compounds of Formula I or Formula II may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The daily dosage of the products of the present invention may be varied over a wide range from 1 to 5000 mg per adult human per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug is ordinarily supplied at a dosage level of from about 0.01 mg/kg to about 200 mg/kg of body weight per day. Particularly, the range is from about 0.03 to about 15 mg/kg of body weight per day, and more particularly, from about 0.05 to about 10 mg/kg of body weight per day. The compound of the present invention may be administered on a regimen up to four or more times per day, preferably of 1 to 2 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

The compounds of the present invention can also be administered in the form of liposome delivery systems, such as small unilamellar vesicles, large unilamellar vesicles, and multilamellar vesicles. Liposomes can be formed from a variety of lipids, including but not limited to amphipathic lipids such as phosphatidylcholines, sphingomyelins, phosphatidylethanolamines, phophatidylcholines, cardiolipins, phosphatidylserines, phosphatidylglycerols, phosphatidic acids, phosphatidylinositols, diacyl trimethylammonium propanes, diacyl dimethylammonium propanes, and stearylamine, neutral lipids such as triglycerides, and combinations thereof. They may either contain cholesterol or may be cholesterol-free.

The compounds of the present invention can also be administered locally. Any delivery device, such as intravascular drug delivery catheters, wires, pharmacological stents and endoluminal paving, may be utilized. The delivery system for such a device may comprise a local infusion catheter that delivers the compound at a rate controlled by the administer.

The present invention provides a drug delivery device comprising an intraluminal medical device, preferably a stent, and a therapeutic dosage of a compound of the invention.

The term “stent” refers to any device capable of being delivered by a catheter. A stent is routinely used to prevent vascular closure due to physical anomalies such as unwanted inward growth of vascular tissue due to surgical trauma. It often has a tubular, expanding lattice-type structure appropriate to be left inside the lumen of a duct to relieve an obstruction. The stent has a lumen wall-contacting surface and a lumen-exposed surface. The lumen-wall contacting surface is the outside surface of the tube and the lumen-exposed surface is the inner surface of the tube. The stent can be polymeric, metallic or polymeric and metallic, and it can optionally be biodegradable.

Commonly, stents are inserted into the lumen in a non-expanded form and are then expanded autonomously, or with the aid of a second device in situ. A typical method of expansion occurs through the use of a catheter-mounted angioplastry balloon which is inflated within the stenosed vessel or body passageway in order to shear and disrupt the obstructions associated with the wall components of the vessel and to obtain an enlarged lumen. Self-expanding stents as described in U.S. Pat. No. 6,776,796 (Falotico et al.) may also be utilized. The combination of a stent with drugs, agents or compounds which prevent inflammation and proliferation, may provide the most efficacious treatment for post-angioplastry restenosis.

Compounds of the invention can be incorporated into or affixed to the stent in a number of ways and in utilizing any number of biocompatible materials. In one exemplary embodiment, the compound is directly incorporated into a polymeric matrix, such as the polymer polypyrrole, and subsequently coated onto the outer surface of the stent. The compound elutes from the matrix by diffusion through the polymer. Stents and methods for coating drugs on stents are discussed in detail in the art. In another exemplary embodiment, the stent is first coated with as a base layer comprising a solution of the compound, ethylene-co-vinylacetate, and polybutylmethacrylate. Then, the stent is further coated with an outer layer comprising only polybutylmethacrylate. The outlayer acts as a diffusion barrier to prevent the compound from eluting too quickly and entering the surrounding tissues. The thickness of the outer layer or topcoat determines the rate at which the compound elutes from the matrix. Stents and methods for coating are discussed in detail in WIPO publication WO9632907, U.S. Publication No. 2002/0016625 and references disclosed therein.

The solution of the compound of the invention and the biocompatible materials/polymers may be incorporated into or onto a stent in a number of ways. For example, the solution may be sprayed onto the stent or the stent may be dipped into the solution. In a preferred embodiment, the solution is sprayed onto the stent and then allowed to dry. In another exemplary embodiment, the solution may be electrically charged to one polarity and the stent electrically changed to the opposite polarity. In this manner, the solution and stent will be attracted to one another. In using this type of spraying process, waste may be reduced and more control over the thickness of the coat may be achieved. Compound is preferably only affixed to the outer surface of the stent which makes contact with one tissue. However, for some compounds, the entire stent may be coated. The combination of the dose of compound applied to the stent and the polymer coating that controls the release of the drug is important in the effectiveness of the drug. The compound preferably remains on the stent for at least three days up to approximately six months and more, preferably between seven and thirty days.

Any number of non-erodible biocompatible polymers may be utilized in conjunction with the compound of the invention. It is important to note that different polymers may be utilized for different stents. For example, the above-described ethylene-co-vinylacetate and polybutylmethacrylate matrix works well with stainless steel stents. Other polymers may be utilized more effectively with stents formed from other materials, including materials that exhibit superelastic properties such as alloys of nickel and titanium.

Methods for introducing a stent into a lumen of a body are well known and the compound-coated stents of this invention are preferably introduced using a catheter. As will be appreciated by those of ordinary skill in the art, methods will vary slightly based on the location of stent implantation. For coronary stent implantation, the balloon catheter bearing the stent is inserted into the coronary artery and the stent is positioned at the desired site. The balloon is inflated, expanding the stent. As the stent expands, the stent contacts the lumen wall. Once the stent is positioned, the balloon is deflated and removed. The stent remains in place with the lumen-contacting surface bearing the compound directly contacting the lumen wall surface. Stent implantation may be accompanied by anticoagulation therapy as needed.

Optimum conditions for delivery of the compounds for use in the stent of the invention may vary with the different local delivery systems used, as well as the properties and concentrations of the compounds used. Conditions that may be optimized include, for example, the concentrations of the compounds, the delivery volume, the delivery rate, the depth of penetration of the vessel wall, the proximal inflation pressure, the amount and size of perforations and the fit of the drug delivery catheter balloon. Conditions may be optimized for inhibition of smooth muscle cell proliferation at the site of injury such that significant arterial blockage due to restenosis does not occur, as measured, for example, by the proliferative ability of the smooth muscle cells, or by changes in the vascular resistance or lumen diameter. Optimum conditions can be determined based on data from animal model studies using routine computational methods.

Another alternative method for administering compounds of this invention may be by conjugating the compound to a targeting agent which directs the conjugate to its intended site of action, i.e., to vascular endothelial cells, or to tumor cells. Both antibody and non-antibody targeting agents may be used. Because of the specific interaction between the targeting agent and its corresponding binding partner, a compound of the present invention can be administered with high local concentrations at or near a target site and thus treats the disorder at the target site more effectively.

The antibody targeting agents include antibodies or antigen-binding fragments thereof, that bind to a targetable or accessible component of a tumor cell, tumor vasculature, or tumor stroma. The “targetable or accessible component” of a tumor cell, tumor vasculature or tumor stroma, is preferably a surface-expressed, surface-accessible or surface-localized component. The antibody targeting agents also include antibodies or antigen-binding fragments thereof, that bind to an intracellular component that is released from a necrotic tumor cell. Preferably such antibodies are monoclonal antibodies, or antigen-binding fragments thereof, that bind to insoluble intracellular antigen(s) present in cells that may be induced to be permeable, or in cell ghosts of substantially all neoplastic and normal cells, but are not present or accessible on the exterior of normal living cells of a mammal.

As used herein, the term “antibody” is intended to refer broadly to any immunologic binding agent such as IgG, IgM, IgA, IgE, F(ab′)2, a univalent fragment such as Fab′, Fab, Dab, as well as engineered antibodies such as recombinant antibodies, humanized antibodies, bispecific antibodies, and the like. The antibody can be either the polyclonal or the monoclonal, although the monoclonal is preferred. There is a very broad array of antibodies known in the art that have immunological specificity for the cell surface of virtually any solid tumor type (see, Summary Table on monoclonal antibodies for solid tumors in U.S. Pat. No. 5,855,866 to Thorpe et al). Methods are known to those skilled in the art to produce and isolate antibodies against tumor (see, U.S. Pat. No.5,855,866 to Thorpe et al., and U.S. Pat. No.6,34,2219 to Thorpe et al.).

Techniques for conjugating therapeutic moiety to antibodies are well known. (See, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985)). Similar techniques can also be applied to attach compounds of the invention to non-antibody targeting agents. Those skilled in the art will know, or be able to determine, methods of forming conjugates with non-antibody targeting agents, such as small molecules, oligopeptides, polysaccharides, or other polyanionic compounds.

Although any linking moiety that is reasonably stable in blood, can be used to link the compounds of the present invention to the targeting agent, biologically-releasable bonds and/or selectively cleavable spacers or linkers are preferred. “Biologically-releasable bonds” and “selectively cleavable spacers or linkers” still have reasonable stability in the circulation, but are releasable, cleavable or hydrolyzable only or preferentially under certain conditions, i.e., within a certain environment, or in contact with a particular agent. Such bonds include, for example, disulfide and trisulfide bonds and acid-labile bonds, as described in U.S. Pat. Nos. 5, 474,765 and 5,762,918 and enzyme-sensitive bonds, including peptide bonds, esters, amides, phosphodiesters and glycosides as described in U.S. Pat. Nos. 5,474,765 and 5,762,918. Such selective-release design features facilitate sustained release of the compounds from the conjugates at the intended target site.

The present invention provides a pharmaceutical composition comprising an effective amount of a compound of the present invention conjugated to a targeting agent and a pharmaceutically acceptable carrier.

The present invention further provides a method of treating of a disorder related to FLT3, particularly a tumor, comprising administering to a subject a therapeutically effective amount of a compound of Formula I or Formula II conjugated to a targeting agent.

When proteins such as antibodies or growth factors, or polysaccharides are used as targeting agents, they are preferably administered in the form of injectable compositions. The injectable antibody solution will be administered into a vein, artery or into the spinal fluid over the course of from 2 minutes to about 45 minutes, preferably from 10 to 20 minutes. In certain cases, intradermal and intracavitary administration are advantageous for tumors restricted to areas close to particular regions of the skin and/or to particular body cavities. In addition, intrathecal administrations may be used for tumors located in the brain.

Therapeutically effective dose of the compound of the present invention conjugated to a targeting agent depends on the individual, the disease type, the disease state, the method of administration and other clinical variables. The effective dosages are readily determinable using data from an animal model. Experimental animals bearing solid tumors are frequently used to optimize appropriate therapeutic doses prior to translating to a clinical environment. Such models are known to be very reliable in predicting effective anti-cancer strategies. For example, mice bearing solid tumors, are widely used in pre-clinical testing to determine working ranges of therapeutic agents that give beneficial anti-tumor effects with minimal toxicity.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A compound selected from the group consisting of Formula I and Formula II: 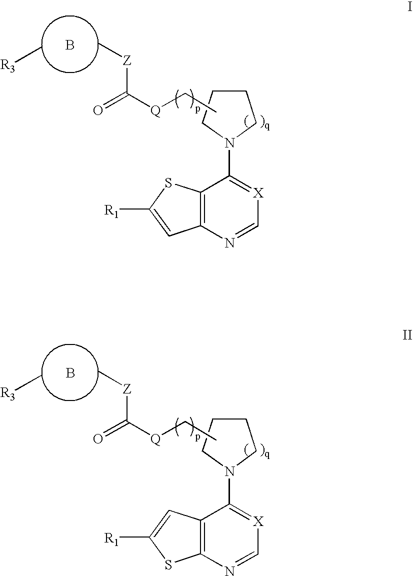
and N-oxides, pharmaceutically acceptable salts, and stereochemical isomers thereof, wherein:
q is 0, 1 or 2;
p is 0 or 1;
Q is NH, N(alkyl), O, or a direct bond;
X is N or CH;
Z is NH, N(alkyl), or CH₂;
B is aryl, cycloalkyl, heteroaryl, or a nine to ten membered benzo-fused heteroaryl;
R₁ is: 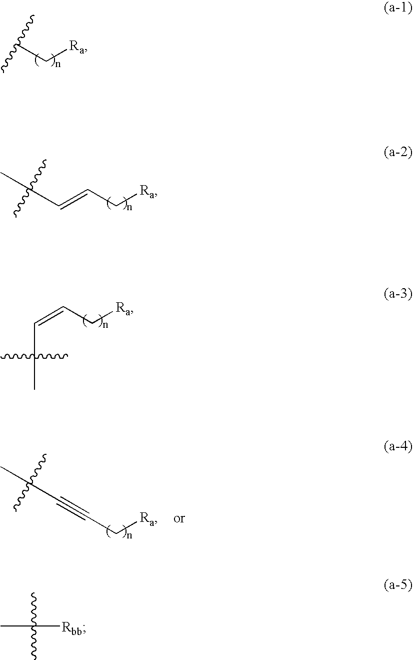
wherein n is 1, 2, 3 or 4;
Rₐ is hydrogen, heteroaryl optionally substituted with R₅, hydroxyl, alkylamino, dialkylamino, oxazolidinonyl optionally substituted with R₅, pyrrolidinonyl optionally substituted with R₅, piperidinonyl optionally substituted with R₅, cyclic heterodionyl optionally substituted with R₅, heterocyclyl optionally substituted with R₅, —COOR_{y}, —CONR_{w}Rₓ, —N(R_{y})CON(R_{w})(Rₓ), —N(R_{w})C(O)ORₓ, —N(R_{w})COR_{y}, —SR_{y}, —SOR_{y}, —SO₂R_{y}, —NR_{w}SO₂R_{y}, —NR_{w}SO₂Rₓ, —SO₃R_{y}, or —OSO₂NRRₓ;
R_{bb} is hydrogen, halogen, aryl, heteroaryl, or heterocyclyl;
R₅ is one, two, or three substituents independently selected from: halogen, cyano, trifluoromethyl, amino, hydroxyl, alkoxy, —C(O)alkyl, —SO₂alkyl, —C(O)N(alkyl)₂, alkyl, —C₍₁₋₄₎alkyl-OH, or alkylamino;
R_{w} and Rₓ are independently selected from: hydrogen, alkyl, alkenyl, aralkyl, or heteroaralkyl, or R_{w} and Rₓ may optionally be taken together to form a 5 to 7 membered ring, optionally containing a heteromoiety selected from O, NH, N(alkyl), SO₂, SO, or S;
R_{y} is selected from: hydrogen, alkyl, alkenyl, cycloalkyl, aryl, aralkyl, heteroaralkyl, or heteroaryl; and
R₃ is one or more substituents, optionally present, and independently selected from: alkyl, alkoxy, halogen, alkoxyether, hydroxyl, thio, nitro, cycloalkyl optionally substituted with R₄, heteroaryl optionally substituted with R₄, alkylamino, heterocyclyl optionally substituted with R₄, partially unsaturated heterocyclyl optionally substituted with R₄, —O(cycloalkyl), pyrrolidinone optionally substituted with R₄, phenoxy optionally substituted with R₄, —CN, —OCHF₂, —OCF₃, —CF₃, halogenated alkyl, heteroaryloxy optionally substituted with R₄, dialkylamino, —NHSO₂alkyl, thioalkyl, or —SO₂alkyl; wherein R₄ is independently selected from: halogen, cyano, trifluoromethyl, amino, hydroxyl, alkoxy, —C(O)alkyl, —CO₂alkyl, —SO₂alkyl, —C(O)N(alkyl)₂, alkyl, or alkylamino.

2. A compound according to claim 1 wherein: R_{w} and Rₓ are independently selected from hydrogen, alkyl, alkenyl, aralkyl, or heteroaralkyl, or may optionally be taken together to form a 5 to 7 membered ring, selected from the group consisting of:

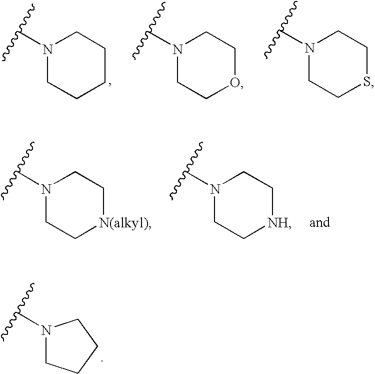

3. A compound according to claim 1 wherein
q is 1 or 2;
X is N; and
B is aryl or heteroaryl.

4. A compound according to claim 3, wherein
Q is NH, O, or a direct bond;
Z is NH or CH₂; and
R₃ is one or more substituents, optionally present, and independently selected from: alkyl, alkoxy, halogen, alkoxyether, cycloalkyl optionally substituted with R₄, alkylamino, heterocyclyl optionally substituted with R₄, -O(cycloalkyl), phenoxy optionally substituted with R₄, dialkylamino, or —SO₂alkyl.

5. A compound according to claim 4, wherein
R₁ is: 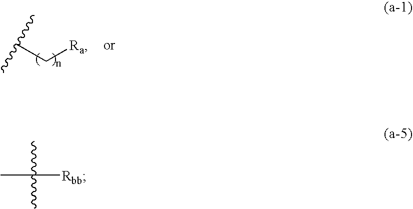
Rₐ is hydrogen, hydroxyl, alkylamino, dialkylamino, heterocyclyl optionally substituted with R₅, —CONR_{w}Rₓ, —N(R_{y})CON(R_{w})(Rₓ), —N(R_{w})C(O)ORₓ, —N(R_{w})COR_{y}, —SO₂R_{y}, —NR_{w}SO₂R_{y}, or —NR_{w}SO₂Rₓ; and
R₃ is one substituentselected from: alkyl, alkoxy, halogen, alkoxyether, cycloalkyl optionally substituted with R₄, alkylamino, heterocyclyl optionally substituted with R₄, -O(cycloalkyl), phenoxy optionally substituted with R₄, dialkylamino, or —SO₂alkyl.

6. A compound according to claim 1 wherein
q is 1 or 2;
p is 0 or 1;
Q is NH, O, or a direct bond;
Z is NH or CH₂;
B is phenyl or pyridyl;
X is N;
R₁ is: 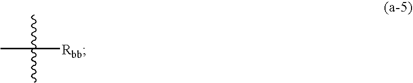
wherein
R_{bb} is hydrogen, halogen, aryl, or heteroaryl; and
R₃ is one substituent selected from: alkyl, alkoxy, heterocyclyl, —O(cycloalkyl), phenoxy, or dialkylamino.

7. A compound according to claim 6, wherein
p is 0;
Q is NH or O;
Z is NH;
R_{bb} is hydrogen; and
R₃ is one substituent selected from: alkyl, -O(cycloalkyl), phenoxy, or dialkylamino.

8. A compound selected from the group consisting of:

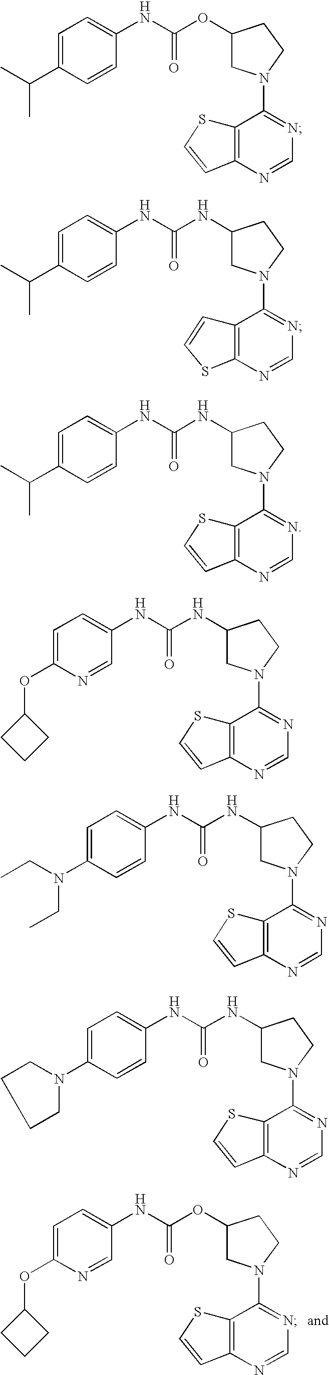

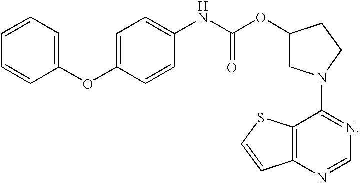

9. A compound selected from the group consisting of:

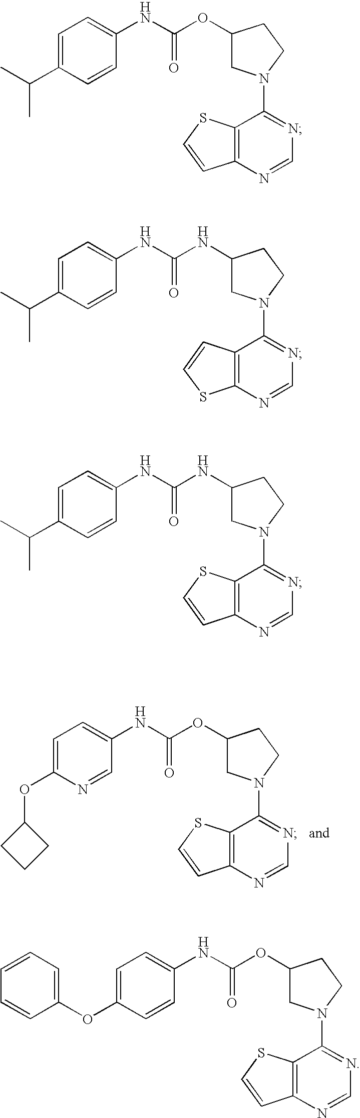

10. A compound according to claim 9, which is:

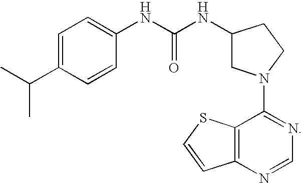

11. A pharmaceutical composition comprising a compound of claim 1 and a pharmaceutically acceptable carrier.

12. (canceled)

13. (canceled)

14. A method for reducing kinase activity of FLT3 in a cell comprising the step of contacting the cell with a compound of claim 1.

15. A method for inhibiting kinase activity of FLT3 in a cell comprising the step of contacting the cell with a compound of claim 1.

16. A method for reducing kinase activity of FLT3 in a subject comprising the step of administering a compound of claim 1 the subject.

17. A method for inhibiting kinase activity of FLT3 in a subject comprising the step of administering a compound of claim 1 the subject.

18. A method for preventing in a subject a disorder related to FLT3 comprising administering to the subject a prophylactically effective amount of a pharmaceutical composition comprising a compound of claim 1 and a pharmaceutically acceptable carrier.

19. A method of treating in a subject a disorder related to FLT3 comprising administering to the subject a therapeutically effective amount of a pharmaceutical composition comprising a compound of claim 1 claims and a pharmaceutically acceptable carrier.

20. The method of claim 18 further comprising administration of a chemotherapeutic agent.

21. The method of claim 18 further comprising administration of gene therapy.

22. The method of claim 18 further comprising administration of immunotherapy.

23. The method of claim 18 further comprising administration of radiation therapy.

24. The method of claim 19 further comprising administration of a chemotherapeutic agent.

25. The method of claim 19 further comprising administration of gene therapy.

26. The method of claim 19 further comprising administration of immunotherapy.

27. The method of claim 19 further comprising administration of radiation therapy.

28. A method for the treatment of a cell proliferative disorder comprising the controlled delivery by release from an intraluminal medical device of a compound of claim 1 in a therapeutically effective amount.

29. A method for the treatment of a disorder related to FLT3 comprising the controlled delivery by release from an intraluminal medical device of a compound of claim 1 in a therapeutically effective amount.

30. The method of claim 28, wherein said intraluminal medical device comprises a stent.

31. The method of claim 29, wherein said intraluminal medical device comprises a stent.

32. A pharmaceutical composition comprising an effective amount of a compound of claim 1 conjugated to a targeting agent and a pharmaceutically acceptable carrier.

33. A method of treating of a cell proliferative disorder comprising administering to a subject a therapeutically effective amount of a compound of claim 1 conjugated to a targeting agent.

34. A method of treating of a disorder related to FLT3 comprising administering to a subject a therapeutically effective amount of a compound of claim 1 conjugated to a targeting agent.

35. A combination of a chemotherapeutic agent and a compound as claimed in claim 1.

36. A process for the preparation of a compound of claim 1 wherein Q is O; said process comprising reacting a compound of Formula V or V′: 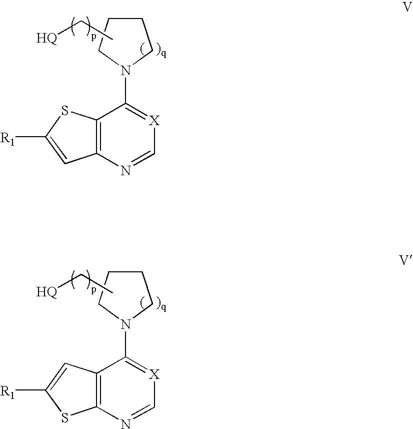
with a compound of Formula VI: 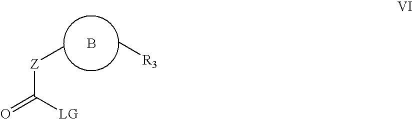
wherein LG comprises a leaving group, in the presence of a base.

37. A process for the preparation of a compound claim 1 wherein Q is O and Z is NH, said process comprising reacting a compound of Formula V or V′: 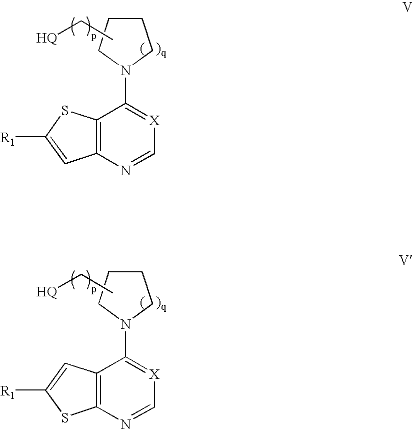
with a compound of the formula R₃BNCO: 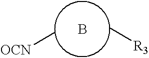
in the presence of a base.

38. l A process for the preparation of a compound of claim 1 wherein Q is NH or N(alkyl), said process comprising reacting a compound of Formula X or X′: 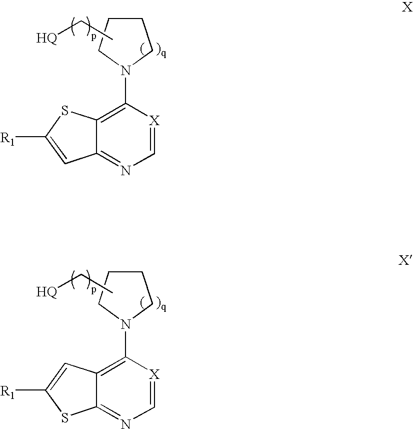
with a compound of Formula VI: 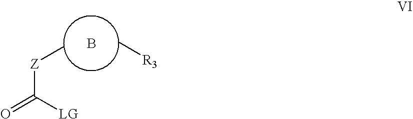
wherein LG comprises as leaving group, in the presence of a base.

39. A process for the preparation of a compound of claim 1 wherein Q is NH or N(alkyl) and Z is NH, said process comprising reacting a compound of Formula X or X′: 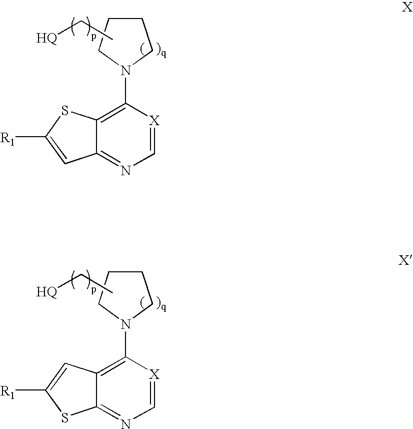
with a compound of the formula R₃BNCO: 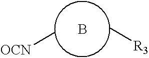
in the presence of a base.

40. A process for the preparation of a compound of claim 1 wherein Q is a direct bond and Z is NH or N(alkyl), said process comprising reacting a compound of Formula XII or XII′: 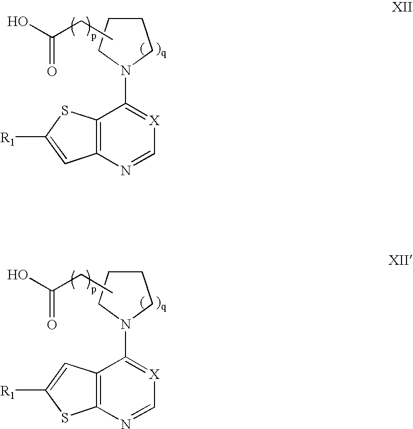
with a compound of the formula R₃BZH: 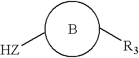
in the presence of a coupling reagent.

41. A process for the preparation of a compound of claim 1 wherein R₁ is R_{bb}, and R_{bb} is aryl or heteroaryl, said process comprising reacting a compound of Formula XIV or XIV′: 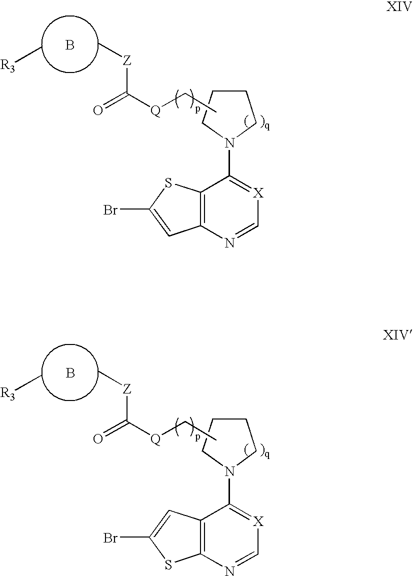
with a compound of the formula: ArB(OR)₂, wherein Ar is aryl or heteroaryl, and R is H or alkyl in the presence of a palladium catalyst.

42. A process for the preparation of a compound of claim 1 wherein R₁ is —CHCH(CH₂)ₙRₐ, said process comprising reacting a compound of Formula XIV or XIV′: 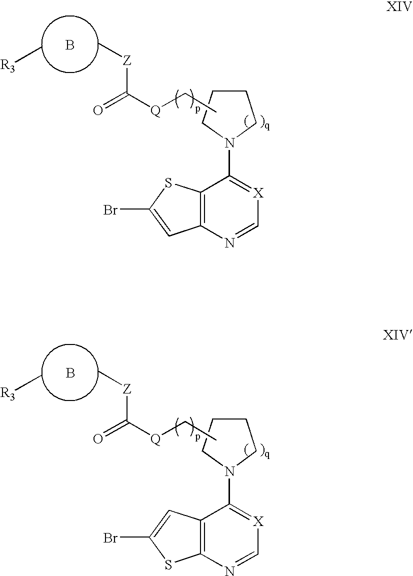
with a compound of Formula XV: 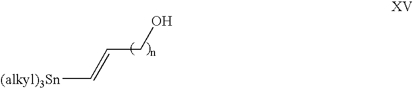
in the presence of a palladium catalyst.

43. A process for the preparation of a compound of claim 1 wherein R₁ is —CC(CH₂)ₙRₐ, said process comprising reacting a compound of Formula XIV or XIV′: 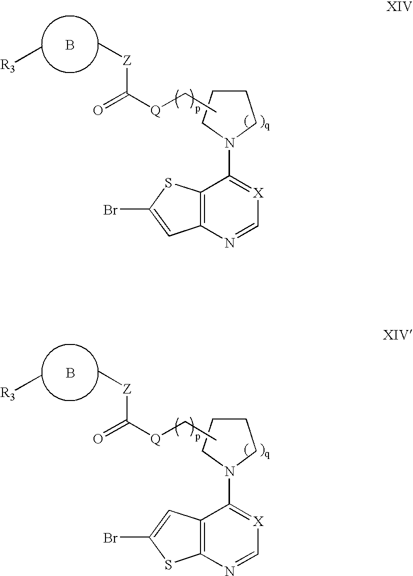
with a compound of the following formula: 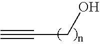
in the presence of a palladium catalyst and a copper catalyst.

44. A pharmaceutical composition comprising the product made by the process of claim 3.

45. A pharmaceutical composition comprising a product made by the process of claim 37.

46. A pharmaceutical composition comprising a product made by the process of claim 38.

47. A pharmaceutical composition comprising a product made by the process of claim 39.

48. A pharmaceutical composition comprising a product made by the process of claim 40.

49. l A pharmaceutical composition comprising a product made by the process of claim 41.

50. A pharmaceutical composition comprising a product made by the process of claim 42.

51. A pharmaceutical composition comprising a product made by the process of claim 43.
